(19) United States
(12) Patent Application Publication
(10) Pub. No.: US 2026/0317394 A1
(43) Pub. Date: Oct. 15, 2026

(54) ANTI-STEAP2 CHIMERIC ANTIGEN RECEPTORS AND USES THEREOF

(71) Applicant: MEDIMMUNE, LLC, Gaithersburg, MD (US)

(72) Inventors: Emily BOSCO, Wilmington, DE (US); Dewald VAN DYK, Wilmington, DE (US); Gordon MOODY, Wilmington, DE (US); Christine FAZENBAKER, Gaithersburg, MD (US); Chien-ying CHANG, Gaithersburg, MD (US)

(21) Appl. No.: 19/719,506

(22) Filed: Jun. 25, 2026

Related U.S. Application Data
(63) Continuation of application No. 18/046,728, filed on Oct. 14, 2022, now Pat. No. 12,686,707.
(60) Provisional application No. 63/262,602, filed on Oct. 15, 2021.

(51) Int. Cl. C07K 14/705; A61P 35/04; C12N 15/86

(52) U.S. Cl. CPC C07K 14/705; A61P 35/04; C12N 15/86; C07K 2319/03; C07K 2319/30

(57) ABSTRACT

The disclosure provides chimeric antigen receptors and antibodies that comprise antigen-binding domains that specifically bind human STEAP2, nucleotides that encode the same, cells comprising the same, and methods of using the same in the treatment of cancer (e.g., prostate cancer).

### CROSS-REFERENCE TO RELATED APPLICATION

This application claims the priority benefit of U.S. Provisional Application No. 63/262,602, filed Oct. 15, 2021, which is incorporated herein by reference in its entirety.

### REFERENCE TO SEQUENCE LISTING SUBMITTED ELECTRONICALLY VIA EFS WEB

The content of the electronically submitted sequence listing (CARTSTEAP2-100-US-NP.xml; Size: 144,479 bytes; and Date of Creation: Oct. 13, 2022) submitted in this application is incorporated herein by reference in its entirety.

### BACKGROUND OF THE DISCLOSURE

Immunotherapy has emerged as a powerful tool in the fight against various types of diseases, including cancer. Immunotherapies harness the power of the patient's own immune system to combat various types of tumors.

Chimeric antigen receptor (CAR) T cell therapy is a specific form of cell-based immunotherapy that uses engineered T cells to fight cancer. In CAR-T cell therapy, T cells are harvested from a patient's blood, engineered ex vivo to express CARs containing both antigen-binding and T cell-activating domains, expanded into a larger population, and administered to the patient. The CAR-T cells act as a living drug, binding to cancer cells and bringing about their destruction. When successful, the effects of CAR-T cell treatment tend to be long lasting, as evidenced by detection of CAR-T cell persistence and expansion in the patients long after clinical remission.

Though several promising CAR-therapies have been approved for use, there remains a need to develop CARs against novel targets to expand the number of indications that can be treated using this therapy. Described herein are novel CARs directed to human STEAP2 and methods of using the same in the treatment of cancer, e.g., prostate cancer.

### SUMMARY OF THE DISCLOSURE

Some aspects of the present disclosure are directed to a polynucleotide comprising a nucleotide sequence encoding a chimeric antigen receptor (CAR), wherein the CAR comprises: (i) an antigen-binding domain that binds an epitope on human six transmembrane epithelial antigen of prostate-2 (STEAP2); (ii) a transmembrane domain; and (iii) an intracellular domain. In some aspects, the antigen-binding domain binds an epitope on an extracellular loop of human STEAP2.

In some aspects, the antigen-binding domain comprises an Fab, Fab′, F(ab′)2, Fd, Fv, single-chain fragment variable (scFv), single chain antibody, V_{H}H, vNAR, nanobody (single-domain antibody), or any combination thereof. In some aspects, the antigen-binding domain comprises a scFv.

In some aspects, the antigen-binding domain comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3.

In some aspects, the antigen-binding domain comprises a VH-CDR3 comprising an amino acid sequence selected from SEQ ID NOs: 6, 16, 26, 36, 46, and 96. In some aspects, the antigen-binding domain comprises a VH-CDR2 comprising an amino acid sequence selected from SEQ ID NOs: 5, 15, 25, 35, 45, and 95. In some aspects, the antigen-binding domain comprises a VH-CDR1 comprising an amino acid sequence selected from SEQ ID NOs: 4, 14, 24, 34, 44, and 94. In some aspects, the antigen-binding domain comprises a VL-CDR3 comprising an amino acid sequence selected from SEQ ID NOs: 3, 13, 23, 33, 43, and 93. In some aspects, the antigen-binding domain comprises a VL-CDR2 comprising an amino acid sequence selected from SEQ ID NOs: 2, 12, 22, 32, 42, and 92. In some aspects, the antigen-binding domain comprises a VL-CDR1 comprising an amino acid sequence selected from SEQ ID NOs: 1, 11, 21, 31, 41, and 91.

In some aspects, the antigen-binding domain comprises: (a) a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (b) a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (c) a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26; (d) a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36; (e) a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46; or (f) a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96.

In some aspects, the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, and 97. In some aspects, the antigen-binding domain comprises a VH comprising an amino acid sequence selected from SEQ ID NOs: 7, 17, 27, 37, 47, and 97.

In some aspects, the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, and 98. In some aspects, the antigen-binding domain comprises a VL comprising an amino acid sequence selected from SEQ ID NOs: 8, 18, 28, 38, 48, and 98.

In some aspects, the antigen-binding domain comprises: (a) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (b) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (c) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28; (d) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; (e) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48; or (f) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57.

In some aspects, the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58.

In some aspects, the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67.

In some aspects, the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68.

In some aspects, the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77.

In some aspects, the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78.

In some aspects, the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87.

In some aspects, the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88.

In some aspects, the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97.

In some aspects, the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, the antigen-binding domain comprises: (a) a VH comprising the amino acid sequence set forth in SEQ ID NO: 7, and a VL comprising the amino acid sequence set forth in SEQ ID NO: 8; (b) a VH comprising the amino acid sequence set forth in SEQ ID NO: 17, and a VL comprising the amino acid sequence set forth in SEQ ID NO: 18; (c) a VH comprising the amino acid sequence set forth in SEQ ID NO: 27, and a VL comprising the amino acid sequence set forth in SEQ ID NO: 28; (d) a VH comprising the amino acid sequence set forth in SEQ ID NO: 37, and a VL comprising the amino acid sequence set forth in SEQ ID NO: 38; (e) a VH comprising the amino acid sequence set forth in SEQ ID NO: 47, and a VL comprising the amino acid sequence set forth in SEQ ID NO: 48; (f) a VH comprising the amino acid sequence set forth in SEQ ID NO: 57, and a VL comprising the amino acid sequence set forth in SEQ ID NO: 58; (g) a VH comprising the amino acid sequence set forth in SEQ ID NO: 67, and a VL comprising the amino acid sequence set forth in SEQ ID NO: 68; (h) a VH comprising the amino acid sequence set forth in SEQ ID NO: 77, and a VL comprising the amino acid sequence set forth in SEQ ID NO: 78; or (i) a VH comprising the amino acid sequence set forth in SEQ ID NO: 87, and a VL comprising the amino acid sequence set forth in SEQ ID NO: 88; or (j) a VH comprising the amino acid sequence set forth in SEQ ID NO: 97, and a VL comprising the amino acid sequence set forth in SEQ ID NO: 98.

In some aspects, the antigen-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9. In some aspects, the antigen-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99.

In some aspects, the intracellular domain comprises a costimulatory domain or a portion thereof. In some aspects, the intracellular domain comprises a costimulatory domain selected from the group consisting of the intracellular domain of CD3z, a CD28 co-stimulatory domain, a CD27 co-stimulatory domain, a 4-1BB co-stimulatory domain, an ICOS co-stimulatory domain, an OX-40 co-stimulatory domain, a GITR co-stimulatory domain, a CD2 co-stimulatory domain, an IL-2Rβ co-stimulatory domain, an MyD88/CD40a CD28 co-stimulatory domain, and any combination thereof. In some aspects, the intracellular domain comprises a 4-1BB co-stimulatory domain. In some aspects, the intracellular domain comprises the intracellular domain of CD3z and a CD28 co-stimulatory domain. In some aspects, the intracellular domain comprises the intracellular domain of CD3z and a 4-1BB co-stimulatory domain. In some aspects, the intracellular domain of CD3z comprises SEQ ID NO: 131 and the 4-1BB co-stimulatory domain comprises SEQ ID NO: 130. In some aspects, the intracellular domain comprising the intracellular domain of CD3z and the 4-1BB co-stimulatory domain comprises SEQ ID NO: 130. In some aspects, the intracellular domain comprises the intracellular domain of CD3z, a CD28 co-stimulatory domain, and a 4-1BB co-stimulatory domain.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10.

In some aspects, the transmembrane domain comprises a transmembrane domain selected from the transmembrane domain of CD4, CD8a, or CD28. In some aspects, the transmembrane domain comprises the transmembrane domain of CD28. In some aspects, the transmembrane domain of CD28 comprises SEQ ID NO: 129.

In some aspects, the CAR further comprises a hinge/spacer domain. In some aspects, the hinge/spacer domain comprises an immunoglobulin hinge/spacer. In some aspects, the hinge/spacer domain comprises an IgG hinge domain. In some aspects, the hinge/spacer domain comprise an IgG1 hinge domain, and IgG2 hinge domain, an IgG3 hinge domain, or an IgG4 hinge domain. In some aspects, the hinge/spacer domain comprises an IgG4 hinge domain. In some aspects, the IgG4 hinge domain comprises SEQ ID NO: 128.

In some aspects, the polynucleotide further encodes an armoring molecule, wherein the armoring molecule counters immunosuppression of a cell in a tumor microenvironment when expressed on a surface of the cell. In some aspects, the armoring molecule comprises a dominant-negative TGF-β receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the armoring molecule comprises the amino acid sequence set forth in SEQ ID NO: 105.

In some aspects, the nucleotide sequence encoding the CAR has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 101 or 103. In some aspects, the nucleotide sequence encoding the CAR comprises the nucleotide sequence set forth in SEQ ID NO: 101 or 103.

In some aspects, the polynucleotide further comprises a second nucleotide sequence encoding an armoring molecule, wherein the second nucleotide sequence has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 104. In some aspects, the second nucleotide comprises the nucleotide sequence set forth in SEQ ID NO: 104. In some aspects, the nucleotide sequence encoding the CAR comprises the nucleotide sequence set forth in SEQ ID NO: 101, and the second nucleotide sequence comprises the nucleotide sequence set forth in SEQ ID NO: 104. In some aspects, the nucleotide sequence encoding the CAR comprises the nucleotide sequence set forth in SEQ ID NO: 103, and the second nucleotide sequence comprises the nucleotide sequence set forth in SEQ ID NO: 104.

In some aspects, the nucleotide sequence encoding the CAR and the second nucleotide sequence are linked by a third nucleotide sequence, wherein the third nucleotide sequence encodes a cleavable peptide linker. In some aspects, the cleavable peptide linker is a self-cleaving peptide linker. In some aspects, the cleavable peptide linker comprises a T2A peptide. In some aspects, the cleavable peptide linker comprises SEQ ID NO: 126.

In some aspects, the polynucleotide comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 107. In some aspects, the polynucleotide comprises the nucleotide sequence set forth in SEQ ID NO: 107.

Some aspects of the present disclosure are directed to a vector or a set of vectors comprising a polynucleotide disclosed herein. In some aspects, the vector is a viral vector.

Some aspects of the present disclosure are directed to a cell comprising a polynucleotide disclosed herein or a vector or a set of vectors disclosed herein. In some aspects, the cell is an immune cell. In some aspects, the cell is selected from the group consisting of a T cell, a Natural Killer (NK) cell, a cytotoxic T lymphocyte (CTL), a regulatory T cell, a tumor infiltrating lymphocyte, and any combination thereof.

Some aspects of the present disclosure are directed to a cell comprising (i) a polynucleotide encoding a chimeric antigen receptor (CAR) that binds human STEAP2 and (ii) a polynucleotide encoding an armoring molecule. In some aspects, the cell is an immune cell. In some aspects, the cell is selected from the group consisting of a T cell, a Natural Killer (NK) cell, a cytotoxic T lymphocyte (CTL), a regulatory T cell, a tumor infiltrating lymphocyte, and any combination thereof. In some aspects, the cell is a human cell.

In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein (a) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6; (b) the VL-CDR1 comprises the amino acid sequence set forth in SEQ ID NO: 11, the VL-CDR2 comprises the amino acid sequence set forth in SEQ ID NO: 12, the VL-CDR3 comprises the amino acid sequence set forth in SEQ ID NO: 13, the VH-CDR1 comprises the amino acid sequence set forth in SEQ ID NO: 14, the VH-CDR2 comprises the amino acid sequence set forth in SEQ ID NO: 15, and the VH-CDR3 comprises the amino acid sequence set forth in SEQ ID NO: 16; (c) the VL-CDR1 comprises the amino acid sequence set forth in SEQ ID NO: 21, the VL-CDR2 comprises the amino acid sequence set forth in SEQ ID NO: 22, the VL-CDR3 comprises the amino acid sequence set forth in SEQ ID NO: 23, the VH-CDR1 comprises the amino acid sequence set forth in SEQ ID NO: 24, the VH-CDR2 comprises the amino acid sequence set forth in SEQ ID NO: 25, and the VH-CDR3 comprises the amino acid sequence set forth in SEQ ID NO: 26; (d) the VL-CDR1 comprises the amino acid sequence set forth in SEQ ID NO: 31, the VL-CDR2 comprises the amino acid sequence set forth in SEQ ID NO: 32, the VL-CDR3 comprises the amino acid sequence set forth in SEQ ID NO: 33, the VH-CDR1 comprises the amino acid sequence set forth in SEQ ID NO: 34, the VH-CDR2 comprises the amino acid sequence set forth in SEQ ID NO: 35, and the VH-CDR3 comprises the amino acid sequence set forth in SEQ ID NO: 36; (e) the VL-CDR1 comprises the amino acid sequence set forth in SEQ ID NO: 41, the VL-CDR2 comprises the amino acid sequence set forth in SEQ ID NO: 42, the VL-CDR3 comprises the amino acid sequence set forth in SEQ ID NO: 43, the VH-CDR1 comprises the amino acid sequence set forth in SEQ ID NO: 44, the VH-CDR2 comprises the amino acid sequence set forth in SEQ ID NO: 45, and the VH-CDR3 comprises the amino acid sequence set forth in SEQ ID NO: 46; or (f) the VL-CDR1 comprises the amino acid sequence set forth in SEQ ID NO: 91, the VL-CDR2 comprises the amino acid sequence set forth in SEQ ID NO: 92, the VL-CDR3 comprises the amino acid sequence set forth in SEQ ID NO: 93, the VH-CDR1 comprises the amino acid sequence set forth in SEQ ID NO: 94, the VH-CDR2 comprises the amino acid sequence set forth in SEQ ID NO: 95, and the VH-CDR3 comprises the amino acid sequence set forth in SEQ ID NO: 96.

In some aspects, (a) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (b) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (c) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28; (d) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; (e) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48; or (f) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, provided is a cell comprising: (a) the VH comprises the amino acid sequence set forth in SEQ ID NO: 7, and the VL comprises the amino acid sequence set forth in SEQ ID NO: 8; (b) the VH comprises the amino acid sequence set forth in SEQ ID NO: 17, and the VL comprises the amino acid sequence set forth in SEQ ID NO: 18; (c) the VH comprises the amino acid sequence set forth in SEQ ID NO: 27, and the VL comprises the amino acid sequence set forth in SEQ ID NO: 28; (d) the VH comprises the amino acid sequence set forth in SEQ ID NO: 37, and the VL comprises the amino acid sequence set forth in SEQ ID NO: 38; (e) the VH comprises the amino acid sequence set forth in SEQ ID NO: 47, and the VL comprises the amino acid sequence set forth in SEQ ID NO: 48; (f) the VH comprises the amino acid sequence set forth in SEQ ID NO: 57, and the VL comprises the amino acid sequence set forth in SEQ ID NO: 58; (g) the VH comprises the amino acid sequence set forth in SEQ ID NO: 67, and the VL comprises the amino acid sequence set forth in SEQ ID NO: 68; (h) the VH comprises the amino acid sequence set forth in SEQ ID NO: 77, and the VL comprises the amino acid sequence set forth in SEQ ID NO: 78; (i) the VH comprises the amino acid sequence set forth in SEQ ID NO: 87, and the VL comprises the amino acid sequence set forth in SEQ ID NO: 88; or (j) the VH comprises the amino acid sequence set forth in SEQ ID NO: 97, and the VL comprises the amino acid sequence set forth in SEQ ID NO: 98.

In some aspects, the armoring molecule comprises a dominant-negative TGF-β receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the armoring molecule comprises the amino acid sequence set forth in SEQ ID NO: 105.

In some aspects, the polynucleotide encoding the CAR comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 101 or 103. In some aspects, the polynucleotide encoding the CAR comprises the nucleotide sequence set forth in SEQ ID NO: 101 or 103.

In some aspects, the polynucleotide encoding the armoring molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 104. In some aspects, the polynucleotide encoding the armoring molecule comprises the nucleotide sequence set forth in SEQ ID NO: 104.

In some aspects, the polynucleotide encoding the CAR comprises the nucleotide sequence set forth in SEQ ID NO: 101, and the polynucleotide encoding the armoring molecule comprises the nucleotide sequence set forth in SEQ ID NO: 104.

In some aspects, the polynucleotide encoding the CAR comprises the nucleotide sequence set forth in SEQ ID NO: 103, and the polynucleotide encoding the armoring molecule comprises the nucleotide sequence set forth in SEQ ID NO: 104.

In some aspects, the polynucleotide encoding the CAR and the polynucleotide encoding the armoring molecule are operably linked under the control of a single promoter. In some aspects, the polynucleotide encoding the CAR and the polynucleotide encoding the armoring molecule are operably linked by an IRES. In some aspects, the polynucleotide encoding the CAR and the polynucleotide encoding the armoring molecule are linked by a nucleotide sequence encoding a cleavable peptide linker. In some aspects, the cleavable peptide linker is a self-cleaving peptide linker. In some aspects, the cleavable peptide linker comprises a T2A peptide. In some aspects, the cleavable peptide linker comprises SEQ ID NO: 126.

Some aspects of the present disclosure are directed to an antibody or an antigen-binding portion thereof that specifically binds human STEAP2, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein (i) the VL-CDR1 comprises an amino acid sequence selected from SEQ ID NOs: 1, 11, 21, 31, 41, and 91; (ii) the VL-CDR2 comprises an amino acid sequence selected from SEQ ID NOs: 2, 12, 22, 32, 42, and 92; (iii) the VL-CDR3 comprises an amino acid sequence selected from SEQ ID NOs: 3, 13, 23, 33, 43, and 93; (iv) the VH-CDR1 comprises an amino acid sequence selected from SEQ ID NOs: 4, 14, 24, 34, 44, and 94; (v) the VH-CDR2 comprises an amino acid sequence selected from SEQ ID NOs: 5, 15, 25, 35, 45, and 95; and (vi) the VH-CDR3 comprises an amino acid sequence selected from SEQ ID NOs: 6, 16, 26, 36, 46, and 96.

In some aspects, (a) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6; (b) the VL-CDR1 comprises the amino acid sequence set forth in SEQ ID NO: 11, the VL-CDR2 comprises the amino acid sequence set forth in SEQ ID NO: 12, the VL-CDR3 comprises the amino acid sequence set forth in SEQ ID NO: 13, the VH-CDR1 comprises the amino acid sequence set forth in SEQ ID NO: 14, the VH-CDR2 comprises the amino acid sequence set forth in SEQ ID NO: 15, and the VH-CDR3 comprises the amino acid sequence set forth in SEQ ID NO: 16; (c) the VL-CDR1 comprises the amino acid sequence set forth in SEQ ID NO: 21, the VL-CDR2 comprises the amino acid sequence set forth in SEQ ID NO: 22, the VL-CDR3 comprises the amino acid sequence set forth in SEQ ID NO: 23, the VH-CDR1 comprises the amino acid sequence set forth in SEQ ID NO: 24, the VH-CDR2 comprises the amino acid sequence set forth in SEQ ID NO: 25, and the VH-CDR3 comprises the amino acid sequence set forth in SEQ ID NO: 26; (d) the VL-CDR1 comprises the amino acid sequence set forth in SEQ ID NO: 31, the VL-CDR2 comprises the amino acid sequence set forth in SEQ ID NO: 32, the VL-CDR3 comprises the amino acid sequence set forth in SEQ ID NO: 33, the VH-CDR1 comprises the amino acid sequence set forth in SEQ ID NO: 34, the VH-CDR2 comprises the amino acid sequence set forth in SEQ ID NO: 35, and the VH-CDR3 comprises the amino acid sequence set forth in SEQ ID NO: 36; (e) the VL-CDR1 comprises the amino acid sequence set forth in SEQ ID NO: 41, the VL-CDR2 comprises the amino acid sequence set forth in SEQ ID NO: 42, the VL-CDR3 comprises the amino acid sequence set forth in SEQ ID NO: 43, the VH-CDR1 comprises the amino acid sequence set forth in SEQ ID NO: 44, the VH-CDR2 comprises the amino acid sequence set forth in SEQ ID NO: 45, and the VH-CDR3 comprises the amino acid sequence set forth in SEQ ID NO: 46; or (f) the VL-CDR1 comprises the amino acid sequence set forth in SEQ ID NO: 91, the VL-CDR2 comprises the amino acid sequence set forth in SEQ ID NO: 92, the VL-CDR3 comprises the amino acid sequence set forth in SEQ ID NO: 93, the VH-CDR1 comprises the amino acid sequence set forth in SEQ ID NO: 94, the VH-CDR2 comprises the amino acid sequence set forth in SEQ ID NO: 95, and the VH-CDR3 comprises the amino acid sequence set forth in SEQ ID NO: 96.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, and 97. In some aspects, the VH comprises an amino acid sequence selected from SEQ ID NOs: 7, 17, 27, 37, 47, 57, 67, 77, 87, and 97.

In some aspects, the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, and 98. In some aspects, the VL comprises an amino acid sequence selected from SEQ ID NOs: 8, 18, 28, 38, 48, 58, 68, 78, 88, and 98.

In some aspects, (a) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (b) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (c) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28; (d) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; (e) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48; (f) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58; (g) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68; (h) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78; (i) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88; or (j) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, (a) the VH comprises the amino acid sequence set forth in SEQ ID NO: 7, and the VL comprises the amino acid sequence set forth in SEQ ID NO: 8; (b) the VH comprises the amino acid sequence set forth in SEQ ID NO: 17, and the VL comprises the amino acid sequence set forth in SEQ ID NO: 18; (c) the VH comprises the amino acid sequence set forth in SEQ ID NO: 27, and the VL comprises the amino acid sequence set forth in SEQ ID NO: 28; (d) the VH comprises the amino acid sequence set forth in SEQ ID NO: 37, and the VL comprises the amino acid sequence set forth in SEQ ID NO: 38; (e) the VH comprises the amino acid sequence set forth in SEQ ID NO: 47, and the VL comprises the amino acid sequence set forth in SEQ ID NO: 48; (f) the VH comprises the amino acid sequence set forth in SEQ ID NO: 57, and the VL comprises the amino acid sequence set forth in SEQ ID NO: 58; (g) the VH comprises the amino acid sequence set forth in SEQ ID NO: 67, and the VL comprises the amino acid sequence set forth in SEQ ID NO: 68; (h) the VH comprises the amino acid sequence set forth in SEQ ID NO: 77, and the VL comprises the amino acid sequence set forth in SEQ ID NO: 78; (i) the VH comprises the amino acid sequence set forth in SEQ ID NO: 87, and the VL comprises the amino acid sequence set forth in SEQ ID NO: 88; or (j) the VH comprises the amino acid sequence set forth in SEQ ID NO: 97, and the VL comprises the amino acid sequence set forth in SEQ ID NO: 98.

Some aspects of the present disclosure are directed to a pharmaceutical composition comprising a polynucleotide disclosed herein, a vector of disclosed herein, a cell disclosed herein, or an antibody or an antigen-binding portion disclosed herein, and a pharmaceutically acceptable excipient.

Some aspects of the present disclosure are directed to a method of treating a disease or condition in a subject in need thereof, comprising administering to the subject a polynucleotide disclosed herein, a vector of disclosed herein, a cell disclosed herein, an antibody or an antigen-binding portion disclosed herein, or a pharmaceutical composition disclosed herein. In some aspects, the disease or condition comprises a cancer.

Some aspects of the present disclosure are directed to a method of treating a cancer in a subject in need thereof, comprising administering to the subject a polynucleotide disclosed herein, a vector of disclosed herein, a cell disclosed herein, an antibody or an antigen-binding portion disclosed herein, or a pharmaceutical composition disclosed herein. In some aspects, the cancer comprises a prostate cancer. In some aspects, the prostate cancer is metastatic, recurrent, or relapsed.

Some aspects of the present disclosure are directed to the use of a polynucleotide disclosed herein, a vector of disclosed herein, a cell disclosed herein, an antibody or an antigen-binding portion disclosed herein, or a pharmaceutical composition disclosed herein in the treatment of a disease or condition in a subject in need thereof. In some aspects, the disease or condition comprises a cancer.

Some aspects of the present disclosure are directed to the use of a polynucleotide disclosed herein, a vector of disclosed herein, a cell disclosed herein, an antibody or an antigen-binding portion disclosed herein, or a pharmaceutical composition disclosed herein in the treatment of a cancer in a subject in need thereof. In some aspects, the cancer comprises a prostate cancer. In some aspects, the prostate cancer is metastatic, recurrent, or relapsed.

### BRIEF DESCRIPTION OF THE DRAWINGS/FIGURES

FIGS. 1A-1C show STEAP2 expression and localization across various tumor types according to the genomic Human Protein Atlas database (FIG. 1A), cDNA array gene expression profiling (FIG. 1), and cell surface protein expression according to immunohistochemistry (IHC) with a custom polyclonal antibody (FIG. 1C). IHC on tissue microarrays containing primary prostate cancer, castrate-resistant prostate cancer (CRPC), and prostate lymph node metastases as well as decalcified full face sections of prostate cancer bone metastases for STEAP2 membrane expression (FIG. 1D) with representative images of STEAP2 in a CRPC (FIG. 1E) and a bone metastasis (FIG. 1F). Summary of STEAP2 IHC and in-situ hybridization (ISH) in normal human tissue microarray and scoring criteria (FIG. 1G). Example of a STEAP2 IHC stain in normal prostate tissue (FIG. 1H).

FIGS. 2A-2F. FIG. 2A shows that the 40A3 scFv-Fc was tested for binding to human STEAP family members (STEAP 1, 2, 3, and 4) and murine STEAP2. The 40A3 scFv-Fc affinity was calculated from the EC50 values on a sigmoidal dose response in Graph Pad Prism and converted to molarity. FIGS. 2B-2F show the results of multiple scFv-Fcs and full-length IgG1 antibodies screened for binding to antigen positive cell lines: Ad293 STEAP3-2 (FIG. 2F), Ad293 STEAP3-2 murine (FIG. 2E), and LNCAP (FIG. 2C) cells and antigen negative cell lines: Ad293 (FIG. 2D) and LNCAP STEAP2 CRISPR (FIG. 2B). STEAP2 expression in the LNCAP cell line “LNCAP STEAP2 CRISPR” has been eliminated via CRISPR knockout, and STEAP3-2 cells are a chimeric cell line with the STEAP2 extracellular loops grafted onto the backbone of the STEAP3 protein to exploit the cell surface localization of STEAP3, yet trigger the immune responses against STEAP2. FIGS. 2B-2F show binding curves for the 40A3 scFv-Fc, 40A3 IgG1, and 14N scFv, a known binder for STEAP2 as a positive control, as well as nonbinding IgG1 as a negative control. Alexa fluor 647 conjugated anti-human Fc secondary antibodies were used for detection of scFv-Fc or IgG1 binding to cells by flow cytometry.

FIGS. 3A-3E. FIG. 3A shows the CAR design including armoring strategy. FIGS. 3B-3D show untransduced (FIG. 3B), 40A3Bz (FIG. 3C), and 40A3Bz dnTGFβRII (FIG. 3D) transduced cell populations at day 10. FIG. 3E shows a western blot of pSMAD 2/3 and total SMAD 2/3 as indicators of acute signaling downstream of the native TGFβRII in 40A3Bz and 40A3Bz dnTGFβRII CAR-T cells. A significant abrogation of TGFβ-mediated signaling in dnTGFβRII (40A3Bz) CAR-T cells compared to 40A3Bz CAR or Untransduced cells alone was confirmed (FIG. 3E).

FIGS. 4A-4N. FIG. 4A shows robust CAR-T expansion after transformation. FIG. 4B-4E show that the CD8+ CAR-T cells maintain a Naïve/Stem-Like Phenotype. FIGS. 4F-4K show 40A3Bz STEAP2 CAR-T cells and 40A3Bz dnTGFβRII STEAP2 CAR-T cells from the same donor cells stained for phenotypic surface markers including CD45RO/CD62L/CD70/CD27 and analyzed using flow cytometry and FlowJo. Key=Naïve (CD45RO−CD62L+), Central memory (CD45RO+CD62L+), Effector Memory (CD45RO+CD62L−) and Effector (CD45RO−CD62L−). CAR=chimeric antigen receptor; dnTGFβRII=dominant-negative transforming growth factor beta receptor II; FACS=fluorescence-activated cell sorting; STEAP2=six transmembrane epithelial antigen of the prostate 2; TGFβ=transforming growth factor beta. FIGS. 4L-4N show that 40A3Bz CAR-T and 40A3Bz dnTGFβRII CAR-T cells display a mixed CD4:CD8 ratio. FIG. 4O shows that dnTGFβRII armoring enables CAR-T activity in the presence of TGFβ as demonstrated by C4-2 tumor cell killing. FIG. 4P shows a range of tumor cell lines profiled by FACS with an anti-STEAP2 antibody-alexa fluor 647 conjugate for antibody binding capacity using the Bang's beads quantum simply cellular kit and assessment of STEAP2 cell surface IHC (FIG. 4P, left panel). The tumor cell lines were co-cultured with 40A3Bz dnTGFβRII CAR-T cells at an E:T ratio of 1:1 and the media analyzed at 24 hours for the levels of IFNγ released from the CAR T-cells. 40A3Bz dnTGFβRII CAR-T induced substantial IFNγ release in co-cultures with C4-2, LNCAP, VCAP, 22RV1 tumor cell lines (FIG. 4P, right panel).

FIGS. 5A-5K. FIGS. 5A-5B and 5D-5E show STEAP2 expression in LNCAP STEAP2 CRISPR (FIGS. 5D-5E) and LNCAP (FIGS. 5A-5B) cellular populations as determined by IHC (FIGS. 5A and 5D) and ISH (FIGS. 5B and 5E). 40A3Bz STEAP2 CAR-T and 40A3Bz dnTGFβRII STEAP2 CAR-T cells were co-cultured with antigen-positive cell lines (“Ad293 STEAP3-2”, and “LNCAP”) and antigen-negative cell lines (“Ad293” cells and “LNCAP STEAP2 CRISPR” cells). Dominant-negative 40A3Bz TGFβRII STEAP2 CAR-T cells expanded in human T-cell media (AIM-V media supplemented with 5% Human AB Heat Inactivated Serum and 300 U/mL IL-2) for 10 days were shown to kill antigen-positive target cells in a similar fashion to unarmored STEAP2 CAR-T cells as shown for LNCAP (FIG. 5C) and Ad293 STEAP3-2 (FIG. 5G), in contrast to LNCAP STEAP2 CRISPR (FIG. 5F) and Ad293 cells (FIG. 5H). Killing of target cells was measured over 100 hours using an xCELLigence impedance assay. FIGS. 5I-5K show supernatants from the same co-culture experiments taken 24 hours post addition of CAR-T cells and cytokines (IFNγ, TNFα, and IL-2) measured using MSD ECL Assay: TNFα (FIG. 5I), IFNγ (FIG. 5J), IL-2 (FIG. 5K).

FIG. 6 shows that the tested STEAP2 CAR-T constructs demonstrate minimal on-target, off-tumor activity as evaluated through IFNγ secretion after 24 hours of coculture.

FIGS. 7A-7R. FIGS. 7A-7C show in vivo results where unarmored STEAP2 CAR-T cells were administered at 3 dose levels (3/7/21×10⁶ cells) via tail vein injection into NSG mice engrafted with prostate cancer cell lines. Tumors were implanted 1:1 in Cultrex BME on the flank. Tumor volume (FIG. 7A) was measured for 35 days post implantation in mice bearing the STEAP2 high expressing cell line C4-2. Mice were randomized when tumors reached 175 mm³ and T cells were administered. Body weight (FIG. 7B) of the mice was measured up to Day 28 post transplantation with the C4-2 cell line. FIG. 7C shows STEAP2 expression as determined by IHC in the C4-2 model. FIGS. 7D-7F show tumor volume measured (FIG. 7D) for 53 days post implantation in mice bearing the cell line 22RV1 with intermediate STEAP2 expression. Mice were randomized when tumors reached 175 mm³ and T cells were administered. Body weight (FIG. 7E) of the mice was measured up to Day 50 post transplantation with the 22RV1 cell line. FIG. 7F shows STEAP2 expression as determined by IHC in the 22RV1 xenograft model. FIG. 7G shows results where 22RV1 tumor bearing mice were bled at Days 4, 7, 14, and 21 post dosing with CAR-T cells. Sera samples were run using an Electrochemiluminescence (ECL) Assay to evaluate cytokine production over time in vivo. FIG. 7H shows a Genevestigator analysis of 40A3Bz murine biodistribution. FIGS. 7I-7K show STEAP2 expression 10 days post infusion as evaluated via ISH, with dose dependent focal infiltration and no signs of damage. FIGS. 7L-7M show no evidence of CD3+ CAR-T infiltration into nerves at the base of heart (21e⁶ cell dose at Day 10 post infusion harvest). FIGS. 7N-7O show no evidence of CD3+ CAR-T infiltration into peripheral subcutaneous nerves (21e⁶ cell dose at Day 10 post infusion harvest). FIGS. 7P-7R show subcutaneous tumor sample analysis after 40A3 CAR-T cells were administered at a concentration of 8e⁶ and evaluated at day 14 post infusion harvest. Two small intact and non-infiltrated peripheral nerves entrapped within the dense CAR-T infiltrate are seen (FIG. 7P, arrows). Also present are several small blood vessels with minimal to mild STEAP2 staining (FIG. 7R, arrows). Despite very dense CD3+ CAR-T infiltrate, the nerves are intact and do not appear to be affected (FIG. 7Q).

FIGS. 8A-8F show that STEAP2 armored CAR-T cells demonstrate superior persistence and differentiation profiles, including with respect to cytolysis and IFN-7 production. FIG. 8A shows a schematic of five rounds of serial killing. FIG. 8B shows phenotypes of T cells following treatment with 40A3Bz dnTGFβRII armored, 40A3Bz unarmored CAR-T cells and control T cells. FIG. 8C shows cytolytic activity of 40A3Bz dnTGFβRII armored, 40A3Bz unarmored CAR-T cells and control T cells. FIG. 8D shows cytokine release of 40A3Bz dnTGFβRII armored, 40A3Bz unarmored CAR-T cells and control T cells FIG. 8E shows tumor volumes and FIG. 8F shows body weight of mice treated with 40A3Bz dnTGFβRII armored, 40A3Bz unarmored CAR-T and control T cells. FIG. 8G shows cytokine release in mice treated with 2.5 e\hat{ }6 40A3Bz dnTGFβRII armored, 40A3B unarmored CAR-T cells and control T cells per mouse. FIG. 8H shows fluorescent signals in mice implanted with C4-2 luciferase expressing tumor cells and treated with untransduced T cells, 40A3Bz dnTGFβRII armored CAR-T cells, or 40A3Bz unarmored CAR-T cells. FIG. 8I shows body weight of the mice measured up to Day 22 post transplantation with the luciferase expressing C4-2 cells.

FIG. 9A-9C show that enhanced CAR-T manufacturing augments anti-tumor activity. FIG. 9A shows 40A3Bz dnTGFβRII CAR-T cells manufactured according to the SMART process and phenotyped at expansion Day 4 as compared to untransduced T cells (UT) from the same donor. FIG. 9B shows tumor volume (top) and body weight (bottom) measured out to 50 days post implantation of 22Rv1 tumor cells overexpressing TGFβ into NSG MHC class 1 class 2 knockout mice treated with 40A3Bz dnTGFβRII SMART CAR-T cells dosed at 4 concentrations (0.3, 1, 3, 6×e6 CAR positive cells). FIG. 9C shows tumor volume (top) and body weight (bottom) of NSG class 1 class 2 knockout mice implanted with PDX fragments from frozen stocks of CTG-3610 prostate cancer cells, randomized when tumor volumes ranged from 125-250 mm³ and dosed as in B with 0.5e6 or 5e6 40A3Bz dnTGFβRII SMART CAR-T cells compared to 5e6 UT SMART controls. The IHC intensity and proportion scores for membrane STEAP2 and TGFβ of the CTG-3610 tumor cells are displayed. FIG. 9D shows tumor volume (top) and body weight (bottom) of NSG class 1 class 2 knockout mice implanted with PDX fragments from frozen stocks of CTG-2440 prostate cancer cells, randomized when tumor volumes ranged from 125-250 mm³ and dosed as in B with 0.5e6 or 5e6 40A3Bz dnTGFβRII SMART CAR-T cells compared to 5e6 UT SMART controls. The IHC intensity and proportion scores for membrane STEAP2 and TGFβ of the CTG-2440 cells are displayed. FIG. 9E shows tumor volume (top) and body weight (bottom) of NSG class 1 class 2 knockout mice implanted with PDX fragments from frozen stocks of Lucap 147 prostate cancer cells, randomized when tumor volumes ranged from 125-250 mm³ and dosed as in B with 0.5e6 or 5e6 40A3Bz dnTGFβRII SMART CAR-T cells compared to 5e6 UT SMART controls. The IHC intensity and proportion scores for membrane STEAP2 and TGFβ of Lucap 147 cells are displayed. FIG. 9F shows tumor volume (top) and body weight (bottom) of NSG class 1 class 2 knockout mice implanted with PDX fragments from frozen stocks of Lucap 73 prostate cancer cells, randomized when tumor volumes ranged from 125-250 mm³ and dosed as in B with 0.5e6 or 5e6 40A3Bz dnTGFβRII SMART CAR-T cells compared to 5e6 UT SMART controls. The IHC intensity and proportion scores for membrane STEAP2 and TGFβ of Lucap 73 cells are displayed.

FIG. 10A shows tumor cell line growth of co-cultures of LNCAP tumor cells and 40A3Bz dnTGFβRII CAR-T cells at E:T ratios of 0.3:1 or untransduced T cells in the presence of blocking anti-STEAP2 antibodies (0.2, 2, 20, or 200 μg/ml). FIG. 10B shows IFNγ levels determined by MSD ECL Assay in supernatants of co-cultures of LNCAP tumor cells and 40A3Bz dnTGFβRII CAR-T cells at E:T ratios of 0.3:1 or 1:1 or untransduced T cells in the presence of blocking anti-STEAP2 antibodies or isotype blocking antibodies (0.2, 2, 20, or 200 μg/ml). FIG. 10C shows tumor cell line growth of co-cultures of LNCAP STEAP2 CRISPR tumor cells and 40A3Bz dnTGFβRII CAR-T cells at E:T ratios of 0.3:1 or untransduced T cells in the presence of blocking anti-STEAP2 antibodies (0.2, 2, 20, or 200 μg/ml). FIG. 10D shows IFNγ levels determined by MSD ECL Assay in supernatants of co-cultures of LNCAP STEAP2 CRISPR tumor cells and 40A3Bz dnTGFβRII CAR-T cells at E:T ratios of 0.3:1 or 1:1 or untransduced T cells in the presence of blocking anti-STEAP2 antibodies or isotype blocking antibodies (0.2, 2, 20, or 200 μg/ml).

### DETAILED DESCRIPTION OF DISCLOSURE

The present disclosure relates to antigen-binding moieties that specifically bind an epitope on human six transmembrane epithelial antigen of prostate-2 (STEAP2). Some aspects of the present disclosure are directed to polynucleotides comprising a nucleotide sequence encoding a chimeric antigen receptor (CAR), wherein the CAR comprises an antigen-binding domain that binds an epitope on human STEAP2. Some aspects of the present disclosure are directed to a host cell comprising the polynucleotide. Other aspects of the present disclosure are directed to antibodies or antigen-binding portions thereof that specifically bind an epitope on human STEAP2. In some aspects, the antigen-binding domain binds an epitope on an extracellular loop of human STEAP2. Further aspects of the present disclosure are directed to methods of treating a subject in need thereof comprising administering the polynucleotide, the cell, and/or the antibody or antigen-binding portion thereof to the subject. In some aspects, the subject is afflicted with a prostate cancer or a tumor derived from a prostate cancer.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. A heavy chain may have the C-terminal lysine or not. Unless specified otherwise herein, the amino acids in the variable regions are numbered using the Kabat numbering system and those in the constant regions are numbered using the EU system.

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the antibodies described herein are of the IgG1 subtype. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen (e.g., human STEAP2). The antigen-binding function of an antibody can be performed by fragments of a full-length antibody. Examples of binding fragments encompassed within the term “antigen-binding portion” of an antibody, e.g., an anti-STEAP2 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “chimeric antigen receptor” or “CAR,” as used herein, refers to an engineered antigen-binding polypeptide, comprising an antigen-binding domain, a transmembrane domain, and an intracellular signaling domain. Expression of a CAR on the surface of a cell, e.g., an immune cell, allows the cell to target and bind a particular antigen. In some aspects, the CAR is expressed by an immune cell, e.g., a T cell. In some aspects, the antigen binding domain comprises an Fab, Fab′, F(ab′)2, Fd, Fv, single-chain fragment variable (scFv), single chain antibody, VHH, vNAR, nanobody (single-domain antibody), or any combination thereof. In some aspects, the transmembrane domain comprises a transmembrane domain selected from the transmembrane domain of CD4, CD8a, or CD28. In some aspects, the intracellular domain comprises a costimulatory domain or a portion thereof. In some aspects, the intracellular domain comprises a costimulatory domain selected from the group consisting of the intracellular domain of CD3z, a CD28 co-stimulatory domain, a CD27 co-stimulatory domain, a 4-1BB co-stimulatory domain, an ICOS co-stimulatory domain, an OX-40 co-stimulatory domain, a GITR co-stimulatory domain, a CD2 co-stimulatory domain, an IL-2Rβ co-stimulatory domain, an MyD88/CD40a CD28 co-stimulatory domain, and any combination thereof. A CAR can further comprise a “hinge” or “spacer” domain. Non-limiting examples of hinge/spacer domains include immunoglobulin hinge/spacer domains, such as an IgG1 hinge domain, and IgG2 hinge domain, an IgG3 hinge domain, or an IgG4 hinge domain.

As used herein, the term “armoring” refers to molecular manipulation of a CAR-expressing cell (e.g., a CAR-T cell) to further express one or more “armoring molecules” that can counter immunosuppression. For example, investigators recently reported modifying CAR-T cells to secrete PD-1-blocking single-chain variable fragments (scFv), which improved CAR-T cell anti-tumor activity in mouse models of PD-L1+ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). Others studies have demonstrated the effectiveness of armoring T cells with a dominant-negative TGF-β receptor type 2 (TGFβRIIDN) armoring molecule to neutralize the suppressive effects of TGF-β on T cells (Bollard et al., Tumor-Specific T-Cells Engineered to Overcome Tumor Immune Evasion Induce Clinical Responses in Patients With Relapsed Hodgkin Lymphoma, J Clin Oncol 36(11):1128-1139 (2018)). Currently, at least one clinical study is investigating the effectiveness of armoring anti-PSMA-CAR-T cells with a TGFβRIIDN armoring molecule for treating castrate-resistant prostate cancer (NCT03089203).

As used herein, the term “affinity” refers to a measure of the strength of the binding of an antigen or target (such as an epitope) to its cognate binding domain (such as a paratope). As used herein, the term “avidity” refers to the overall stability of the complex between a population of epitopes and paratopes (i.e., antigens and antigen binding domains).

The term “epitope” refers to a site on an antigen (e.g., STEAP2) to which a chimeric antigen receptor, immunoglobulin, or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “binds to the same epitope” with reference to two or more antigen-binding moieties means that the antigen-binding moieties bind to the same segment of amino acid residues. Antigen-binding moieties that “compete with another antibody for binding to a target” refer to antigen-binding moieties that inhibit (partially or completely) the binding of the other antibody to the target.

As used herein, the terms “specific binding,” “selective binding,” “selectively binds,” and “specifically binds,” refer to an antigen-binding moiety (e.g., a CAR or an antibody) binding to an epitope on a predetermined antigen. Typically, the antigen-binding moiety (e.g., a CAR or an antibody) (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., human STEAP2, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antigen-binding moiety (e.g., a CAR or an antibody) that “specifically binds to human STEAP2” refers to an antigen-binding moiety (e.g., a CAR or an antibody) that binds to human STEAP2 with a K_{D} of 10⁻⁷M or less, such as approximately less than 10⁻⁸ M, 10⁻⁹ M or 10⁻¹⁰ M or even lower.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in a STEAP2-binding moiety (e.g., an anti-STEAP2 CAR or antibody) is replaced with another amino acid residue from the same side chain family.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

“Immunotherapy” refers to the treatment of a subject afflicted with, or at risk of contracting or suffering a recurrence of, a disease by a method comprising inducing, enhancing, suppressing or otherwise modifying the immune system or an immune response.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, “cancer” refers a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream.

As used herein, the term an “effective amount” or a “therapeutically effective amount” of an administered therapeutic substance, such as a CAR-T cell, is an amount sufficient to carry out a specifically stated or intended purpose, such as treating or treatment of cancer. An “effective amount” can be determined empirically in a routine manner in relation to the stated purpose.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the terms “ug” and “uM” are used interchangeably with “μg” and “μM,” respectively.

Various aspects described herein are described in further detail in the following subsections.

### I. Polynucleotides of the Disclosure

Some aspects of the present disclosure are directed to polynucleotides comprising a nucleotide sequence encoding a CAR that specifically binds human STEAP2. In some aspects, the CAR comprises (i) an antigen-binding domain that binds an epitope on STEAP2, (ii) a transmembrane domain, and (iii) an intracellular domain. In some aspects, the CAR further comprises a hinge/spacer domain. In some aspects, the hinge/spacer domain is positioned between the antigen-binding domain and the transmembrane domain.

In some aspects, the polynucleotide further comprises a nucleotide sequence encoding an armoring molecule. In some aspects, the nucleotide sequence encoding the CAR and the nucleotide sequence encoding the armoring moiety are expressed under the control of the same promoter. In some aspects, the nucleotide sequence encoding the CAR and the nucleotide sequence encoding the armoring moiety are expressed under the control of two promoter. In some aspects, the two promoters are different promoters. In some aspects, the nucleotide sequence encoding the CAR and the nucleotide sequence encoding the armoring moiety are expressed as a single contiguous polypeptide. In some aspects, the nucleotide sequence encoding the CAR and the nucleotide sequence encoding the armoring moiety are expressed as two separate polypeptides. In some aspects, the CAR and the nucleotide sequence encoding the armoring moiety are linked by a nucleotide sequence encoding a linker. In some aspects, the linker is a peptide linker. In some aspects, the linker is a cleavable linker. In some aspects, the linker is a self-cleaving peptide linker, e.g., comprising a T2A peptide.

### I.A. Antigen-Binding Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, and (iii) a transmembrane domain. Any antigen-binding domain can be used in the compositions disclosed herein. In some aspects, the antigen-binding domain comprises an Fab, Fab′, F(ab′)2, Fd, Fv, single-chain fragment variable (scFv), single chain antibody, VHH, vNAR, nanobody (single-domain antibody), or any combination thereof. In some aspects, the antigen-binding domain comprises a scFv.

In some aspects, the antigen-binding domain of the CAR comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3. In some aspects, the antigen-binding domain comprises a VH-CDR3 comprising an amino acid sequence selected from SEQ ID NOs: 6, 16, 26, 36, 46, and 96. In some aspects, the antigen-binding domain comprises a VH-CDR2 comprising an amino acid sequence selected from SEQ ID NOs: 5, 15, 25, 35, 45, and 95. In some aspects, the antigen-binding domain comprises a VH-CDR1 comprising an amino acid sequence selected from SEQ ID NOs: 4, 14, 24, 34, 44, and 94.

In some aspects, the antigen-binding domain comprises a VL-CDR3 comprising an amino acid sequence selected from SEQ ID NOs: 3, 13, 23, 33, 43, and 93. In some aspects, wherein the antigen-binding domain comprises a VL-CDR2 comprising an amino acid sequence selected from SEQ ID NOs: 2, 12, 22, 32, 42, and 92. In some aspects, the antigen-binding domain comprises a VL-CDR1 comprising an amino acid sequence selected from SEQ ID NOs: 1, 11, 21, 31, 41, and 91.

In some aspects, the antigen binding domain comprises a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the antigen binding domain comprises a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16.

In some aspects, the antigen binding domain comprises a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26.

In some aspects, the antigen binding domain comprises a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36.

In some aspects, the antigen binding domain comprises a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46.

In some aspects, the antigen binding domain comprises the VL-CDR1, VL-CDR2, and VL-CDR3 present in the VL region having the amino acid sequence set forth in SEQ ID NO: 58; and the VH-CDR1, VH-CDR2, and VH-CDR3 present in the VH region having the amino acid sequence set forth in SEQ ID NO: 57.

In some aspects, the antigen binding domain comprises the VL-CDR1, VL-CDR2, and VL-CDR3 present in the VL region having the amino acid sequence set forth in SEQ ID NO: 68; and the VH-CDR1, VH-CDR2, and VH-CDR3 present in the VH region having the amino acid sequence set forth in SEQ ID NO: 67.

In some aspects, the antigen binding domain comprises the VL-CDR1, VL-CDR2, and VL-CDR3 present in the VL region having the amino acid sequence set forth in SEQ ID NO: 78; and the VH-CDR1, VH-CDR2, and VH-CDR3 present in the VH region having the amino acid sequence set forth in SEQ ID NO: 77.

In some aspects, the antigen binding domain comprises the VL-CDR1, VL-CDR2, and VL-CDR3 present in the VL region having the amino acid sequence set forth in SEQ ID NO: 88; and the VH-CDR1, VH-CDR2, and VH-CDR3 present in the VH region having the amino acid sequence set forth in SEQ ID NO: 87.

In some aspects, the antigen binding domain comprises the VL-CDR1, VL-CDR2, and VL-CDR3 present in the VL region having the amino acid sequence set forth in SEQ ID NO: 98; and the VH-CDR1, VH-CDR2, and VH-CDR3 present in the VH region having the amino acid sequence set forth in SEQ ID NO: 97.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, and 97. In some aspects, the antigen-binding domain comprises a VH comprising an amino acid sequence selected from SEQ ID NOs: 7, 17, 27, 37, 47, 57, 67, 77, 87, and 97.

In some aspects, the CAR comprises an antigen-binding domain comprising a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, and 98. In some aspects, the antigen-binding domain comprises a VL comprising an amino acid sequence selected from SEQ ID NOs: 8, 18, 28, 38, 48, 58, 68, 78, 88, and 98.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the CAR comprises an antigen-binding domain comprising a VH comprising the amino acid sequence set forth in SEQ ID NO: 7, and a VL comprising the amino acid sequence set forth in SEQ ID NO: 8.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the CAR comprises an antigen-binding domain comprising a VH comprising the amino acid sequence set forth in SEQ ID NO: 17, and a VL comprising the amino acid sequence set forth in SEQ ID NO: 18.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28. In some aspects, the CAR comprises an antigen-binding domain comprising a VH comprising the amino acid sequence set forth in SEQ ID NO: 27, and a VL comprising the amino acid sequence set forth in SEQ ID NO: 28.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38. In some aspects, the CAR comprises an antigen-binding domain comprising a VH comprising the amino acid sequence set forth in SEQ ID NO: 37, and a VL comprising the amino acid sequence set forth in SEQ ID NO: 38.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48. In some aspects, the CAR comprises an antigen-binding domain comprising a VH comprising the amino acid sequence set forth in SEQ ID NO: 47, and a VL comprising the amino acid sequence set forth in SEQ ID NO: 48.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58. In some aspects, the CAR comprises an antigen-binding domain comprising a VH comprising the amino acid sequence set forth in SEQ ID NO: 57, and a VL comprising the amino acid sequence set forth in SEQ ID NO: 58.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68. In some aspects, the CAR comprises an antigen-binding domain comprising a VH comprising the amino acid sequence set forth in SEQ ID NO: 67, and a VL comprising the amino acid sequence set forth in SEQ ID NO: 68.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78. In some aspects, the CAR comprises an antigen-binding domain comprising a VH comprising the amino acid sequence set forth in SEQ ID NO: 77, and a VL comprising the amino acid sequence set forth in SEQ ID NO: 78.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88. In some aspects, the CAR comprises an antigen-binding domain comprising a VH comprising the amino acid sequence set forth in SEQ ID NO: 87, and a VL comprising the amino acid sequence set forth in SEQ ID NO: 88.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the CAR comprises an antigen-binding domain comprising a VH comprising the amino acid sequence set forth in SEQ ID NO: 97, and a VL comprising the amino acid sequence set forth in SEQ ID NO: 98.

In some aspects, the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9. In some aspects, the CAR comprises an antigen-binding domain comprising the amino acid sequence set forth in SEQ ID NO: 9.

In some aspects, the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99. In some aspects, the CAR comprises an antigen-binding domain comprising the amino acid sequence set forth in SEQ ID NO: 99.

### I.B. Intracellular Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, and (iii) a transmembrane domain. Any intracellular signaling domain can be used in the compositions disclosed herein. In some aspects, the intracellular signaling domain comprises a costimulatory domain or a portion thereof.

In some aspects, the intracellular domain comprises a costimulatory domain selected from the group consisting of the intracellular domain of CD3z, a CD28 co-stimulatory domain, a CD27 co-stimulatory domain, a 4-1BB co-stimulatory domain, an ICOS co-stimulatory domain, an OX-40 co-stimulatory domain, a GITR co-stimulatory domain, a CD2 co-stimulatory domain, an IL-2Rβ co-stimulatory domain, an MyD88/CD40a CD28 co-stimulatory domain, and any combination thereof.

In some aspects, the intracellular domain comprises a 4-1BB co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) a 4-1BB costimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) a 4-1BB co-stimulatory domain comprising the amino acid sequence set forth in SEQ ID NO: 130.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; and (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising the amino acid sequence set forth in SEQ ID NO: 9; and (ii) a 4-1BB co-stimulatory domain comprising the amino acid sequence set forth in SEQ ID NO: 130.

In some aspects, the intracellular domain comprises the intracellular domain of CD3z and a CD28 co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) the intracellular domain of CD3z and a CD28 co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) the intracellular domain of CD3z comprising the amino acid sequence set forth in SEQ ID NO: 131 and a CD28 co-stimulatory domain comprising the amino acid sequence set forth in SEQ ID NO: 133.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133. In some aspects, the CAR comprises (i) an antigen-binding domain comprising the amino acid sequence set forth in SEQ ID NO: 9; and (ii) the intracellular domain of CD3z comprising the amino acid sequence set forth in SEQ ID NO: 131 and a CD28 co-stimulatory domain comprising the amino acid sequence set forth in SEQ ID NO: 133.

In some aspects, the intracellular domain comprises the intracellular domain of CD3z and a 4-1BB co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) the intracellular domain of CD3z and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) the intracellular domain of CD3z comprising the amino acid sequence set forth in SEQ ID NO: 131 and a 4-1BB co-stimulatory domain comprising the amino acid sequence set forth in SEQ ID NO: 130.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising the amino acid sequence set forth in SEQ ID NO: 9; and (ii) the intracellular domain of CD3z comprising the amino acid sequence set forth in SEQ ID NO: 131 and a 4-1BB co-stimulatory domain: comprising the amino acid sequence set forth in SEQ ID NO: 130.

In some aspects, the intracellular domain comprises the intracellular domain of CD3z, a CD28 co-stimulatory domain, and a 4-1BB co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) the intracellular domain of CD3z, a CD28 co-stimulatory domain, and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising the amino acid sequence set forth in SEQ ID NO: 9; and (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130.

In some aspects, the intracellular domain comprises a 4-1BB co-stimulatory domain. In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (ii) a 4-1BB costimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; and (ii) a 4-1BB co-stimulatory domain comprising the amino acid sequence set forth in SEQ ID NO: 130.

In some aspects, the intracellular domain comprises the intracellular domain of CD3z and a CD28 co-stimulatory domain. In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (ii) the intracellular domain of CD3z and a CD28 co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; and (ii) the intracellular domain of CD3z comprising the amino acid sequence set forth in SEQ ID NO: 131 and a CD28 co-stimulatory domain comprising the amino acid sequence set forth in SEQ ID NO: 133.

In some aspects, the intracellular domain comprises the intracellular domain of CD3z and a 4-1BB co-stimulatory domain. In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (ii) the intracellular domain of CD3z and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; and (ii) the intracellular domain of CD3z comprising the amino acid sequence set forth in SEQ ID NO: 131 and a 4-1BB co-stimulatory domain comprising the amino acid sequence set forth in SEQ ID NO: 130.

In some aspects, the intracellular domain comprises the intracellular domain of CD3z, a CD28 co-stimulatory domain, and a 4-1BB co-stimulatory domain. In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (ii) the intracellular domain of CD3z, a CD28 co-stimulatory domain, and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; and (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130.

In some aspects, the intracellular domain comprises a 4-1BB co-stimulatory domain. In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (ii) a 4-1BB costimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98 and (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; and (ii) a 4-1BB co-stimulatory domain comprising the amino acid sequence set forth in SEQ ID NO: 130.

In some aspects, the intracellular domain comprises the intracellular domain of CD3z and a CD28 co-stimulatory domain. In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (ii) the intracellular domain of CD3z and a CD28 co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; and (ii) the intracellular domain of CD3z comprising the amino acid sequence set forth in SEQ ID NO: 131 and a CD28 co-stimulatory domain comprising the amino acid sequence set forth in SEQ ID NO: 133.

In some aspects, the intracellular domain comprises the intracellular domain of CD3z and a 4-1BB co-stimulatory domain. In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (ii) the intracellular domain of CD3z and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; and (ii) the intracellular domain of CD3z comprising the amino acid sequence set forth in SEQ ID NO: 131 and a 4-1BB co-stimulatory domain comprising the amino acid sequence set forth in SEQ ID NO: 130.

In some aspects, the intracellular domain comprises the intracellular domain of CD3z, a CD28 co-stimulatory domain, and a 4-1BB co-stimulatory domain. In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; and (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the CAR comprises the amino acid sequence set forth in SEQ ID NO: 10.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 108. In some aspects, the CAR comprises the amino acid sequence set forth in SEQ ID NO: 108.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 109. In some aspects, the CAR comprises the amino acid sequence set forth in SEQ ID NO: 109.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 110. In some aspects, the CAR comprises the amino acid sequence set forth in SEQ ID NO: 110.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 111. In some aspects, the CAR comprises the amino acid sequence set forth in SEQ ID NO: 111.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 112. In some aspects, the CAR comprises the amino acid sequence set forth in SEQ ID NO: 112.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 113. In some aspects, the CAR comprises the amino acid sequence set forth in SEQ ID NO: 113.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 114. In some aspects, the CAR comprises the amino acid sequence set forth in SEQ ID NO: 114.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 115. In some aspects, the CAR comprises the amino acid sequence set forth in SEQ ID NO: 115.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 116. In some aspects, the CAR comprises the amino acid sequence set forth in SEQ ID NO: 116.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 118. In some aspects, the CAR comprises the amino acid sequence set forth in SEQ ID NO: 118.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 119. In some aspects, the CAR comprises the amino acid sequence set forth in SEQ ID NO: 119.

### I.C. Transmembrane Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, and (iii) a transmembrane domain. Any transmembrane domain can be used in the compositions disclosed herein. In some aspects, the transmembrane domain comprises a transmembrane domain selected from the transmembrane domain of CD4, CD8a, or CD28. In some aspects, the transmembrane domain comprises a CD28 transmembrane domain.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) a 4-1BB costimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising the amino acid sequence set forth in SEQ ID NO: 9; (ii) a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a CD28 co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 131 and a CD28 co-stimulatory domain comprising the amino acid sequence set forth in SEQ ID NO: 133; and (iii) a transmembrane domain comprising the amino acid sequence set forth in SEQ ID NO: 129.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; and (iii) a transmembrane domain comprising the transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising the amino acid sequence set forth in SEQ ID NO: 9; (ii) the intracellular domain of CD3z comprising the amino acid sequence set forth in SEQ ID NO: 131 and a CD28 co-stimulatory domain comprising the amino acid sequence set forth in SEQ ID NO: 133; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 131 and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising the amino acid sequence set forth in SEQ ID NO: 9; (ii) the intracellular domain of CD3z comprising the amino acid sequence set forth in SEQ ID NO: 131 and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z, a CD28 co-stimulatory domain, and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising the amino acid sequence set forth in SEQ ID NO: 9; (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) a 4-1BB costimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) the intracellular domain of CD3z and a CD28 co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) the intracellular domain of CD3z comprising the amino acid sequence set forth in SEQ ID NO: 131 and a CD28 co-stimulatory domain comprising the amino acid sequence set forth in SEQ ID NO: 133; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) the intracellular domain of CD3z and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) the intracellular domain of CD3z comprising the amino acid sequence set forth in SEQ ID NO: 131 and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) the intracellular domain of CD3z, a CD28 co-stimulatory domain, and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) a 4-1BB costimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) the intracellular domain of CD3z and a CD28 co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 97 and a VL comprising the amino acid sequence set forth in SEQ ID NO: 18; (ii) the intracellular domain of CD3z comprising the amino acid sequence set forth in SEQ ID NO: 131 and a CD28 co-stimulatory domain comprising the amino acid sequence set forth in SEQ ID NO: 133; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) the intracellular domain of CD3z and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) the intracellular domain of CD3z comprising the amino acid sequence set forth in SEQ ID NO: 131 and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) the intracellular domain of CD3z, a CD28 co-stimulatory domain, and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130; and (iii) a transmembrane domain comprising the amino acid sequence set forth in SEQ ID NO: 129.

In some aspects, the CAR comprises an amino acid sequence set forth in SEQ ID NO: 10. In some aspects, the CAR comprises an amino acid sequence set forth in SEQ ID NO: 108. In some aspects, the CAR comprises an amino acid sequence set forth in SEQ ID NO: 109. In some aspects, the CAR comprises an amino acid sequence set forth in SEQ ID NO: 110. In some aspects, the CAR comprises an amino acid sequence set forth in SEQ ID NO: 111. In some aspects, the CAR comprises an amino acid sequence set forth in SEQ ID NO: 112. In some aspects, the CAR comprises an amino acid sequence set forth in SEQ ID NO: 113. In some aspects, the CAR comprises an amino acid sequence set forth in SEQ ID NO: 114. In some aspects, the CAR comprises an amino acid sequence set forth in SEQ ID NO: 115. In some aspects, the CAR comprises an amino acid sequence set forth in SEQ ID NO: 116. In some aspects, the CAR comprises an amino acid sequence set forth in SEQ ID NO: 118. In some aspects, the CAR comprises an amino acid sequence set forth in SEQ ID NO: 119.

### I.D. Spacer/Hinge Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, (iii) a transmembrane domain, and (iv) a hinge/spacer domain. Any hinge/spacer domain can be used in the compositions disclosed herein. In some aspects, the hinge/spacer domain comprises a human immunoglobulin hinge/spacer domain. In some aspects, the hinge/spacer domain comprises an IgG hinge domain. In some aspects, the hinge/spacer domain comprise an IgG1 hinge domain, and IgG2 hinge domain, an IgG3 hinge domain, or an IgG4 hinge domain. In some aspects, the hinge/spacer domain comprises an IgG4 hinge domain. In some aspects, the IgG hinge domain is a variant hinge domain. In some aspects, the IgG4 hinge domain is a variant IgG4 hinge domain. In some aspects, the variant IgG4 hinge domain comprises a S228P mutation. In some aspects, the IgG4 hinge domain comprises an amino acid sequence set forth in SEQ ID NO: 128.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) a 4-1BB costimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129 and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) a 4-1BB co-stimulatory domain comprising the amino acid sequence set forth in SEQ ID NO: 130; (iii) a transmembrane domain comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising the amino acid sequence set forth in SEQ ID NO: 9; (ii) a 4-1BB co-stimulatory domain comprising the amino acid sequence set forth in SEQ ID NO: 130; (iii) a transmembrane domain comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a CD28 co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 131 and a CD28 co-stimulatory domain comprising the amino acid sequence set forth in SEQ ID NO: 133; (iii) a transmembrane comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising the amino acid sequence set forth in SEQ ID NO: 9; (ii) the intracellular domain of CD3z and a CD28 co-stimulatory domain; (iii) a transmembrane domain comprising the transmembrane domain of CD28; and (iv) an IgG hinge domain.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising the amino acid sequence set forth in SEQ ID NO: 9; (ii) the intracellular domain of CD3z and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising the amino acid sequence set forth in SEQ ID NO: 9; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) a 4-1BB costimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) a 4-1BB co-stimulatory domain comprising the amino acid sequence set forth in SEQ ID NO: comprising the amino acid sequence set forth in SEQ ID NO: 131; (iii) a transmembrane domain comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) the intracellular domain of CD3z and a CD28 co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) the intracellular domain of CD3z comprising the amino acid sequence set forth in SEQ ID NO: 131 and a CD28 co-stimulatory domain comprising the amino acid sequence set forth in SEQ ID NO: 133; (iii) a transmembrane domain comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) the intracellular domain of CD3z and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:128. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) the intracellular domain of CD3z and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 17 and a VL comprising the amino acid sequence set forth in SEQ ID NO: 18; (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130; (iii) a transmembrane comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) a 4-1BB costimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) a 4-1BB co-stimulatory domain comprising the amino acid sequence set forth in SEQ ID NO: 130; (iii) a transmembrane domain comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) the intracellular domain of CD3z and a CD28 co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) the intracellular domain of CD3z comprising the amino acid sequence set forth in SEQ ID NO: 131 and a CD28 co-stimulatory domain comprising the amino acid sequence set forth in SEQ ID NO: 133; (iii) a transmembrane domain comprising the transmembrane domain of CD28 comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) the intracellular domain of CD3z and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) the intracellular domain of CD3z comprising the amino acid sequence set forth in SEQ ID NO: 131 and a 4-1BB co-stimulatory domain comprising the amino acid sequence set forth in SEQ ID NO: 130; (iii) a transmembrane domain comprising the amino acid sequence set forth in SEQ ID NO: 129; and (iv) an IgG hinge domain comprising the amino acid sequence set forth in SEQ ID NO: 128.

In some aspects, the CAR comprises (i) an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 131, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 133, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 129; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 128. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) the intracellular domain of CD3z comprising the amino acid sequence set forth in SEQ ID NO: 131, a CD28 co-stimulatory domain comprising the amino acid sequence set forth in SEQ ID NO: 133, and a 4-1BB co-stimulatory domain comprising the amino acid sequence set forth in SEQ ID NO: 130; (iii) a transmembrane domain comprising the amino acid sequence set forth in SEQ ID NO: 129; and (iv) an IgG hinge domain comprising the amino acid sequence set forth in SEQ ID NO:128. In some aspects, the CAR comprises (i) an antigen-binding domain comprising a VH comprising the amino acid sequence set forth in SEQ ID NO: 97 and a VL comprising the amino acid sequence set forth in SEQ ID NO: 98; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain.

In some aspects, the nucleotide sequence encoding the CAR has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 101. In some aspects, the nucleotide sequence encoding the CAR comprises the nucleotide sequence set forth in SEQ ID NO: 101.

In some aspects, the nucleotide sequence encoding the CAR has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 102. In some aspects, the nucleotide sequence encoding the CAR comprises the nucleotide sequence set forth in SEQ ID NO: 102.

In some aspects, the nucleotide sequence encoding the CAR has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 103. In some aspects, the nucleotide sequence encoding the CAR comprises the nucleotide sequence set forth in SEQ ID NO: 103.

### I.E. Armoring Molecule

Disclosed herein are polynucleotides comprising (a) a nucleotide sequence encoding a CAR, wherein the CAR comprises an antigen-binding domain that binds an epitope on human STEAP2, and (b) a nucleotide sequence encoding an armoring molecule. One approach to making CAR-T cells that are more resistant to tumor-associated immunosuppression is called “armoring.” Armoring is the molecular manipulation of a CAR-T cell to express one or more “armoring molecules” that can counter immunosuppression. For example, investigators reported modifying CAR-T cells to secrete PD-1-blocking single-chain variable fragments (scFv), which improved CAR-T cell anti-tumor activity in mouse models of PD-L1+ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). Others studies have demonstrated the effectiveness of armoring T cells with a dominant-negative TGF-β receptor type 2 (TGFβRIIDN) armoring molecule to neutralize the suppressive effects of TGF-β on T cells (Bollard et al., Tumor-Specific T-Cells Engineered to Overcome Tumor Immune Evasion Induce Clinical Responses in Patients With Relapsed Hodgkin Lymphoma, J Clin Oncol 36(11):1128-1139 (2018)). Currently, at least one clinical study is investigating the effectiveness of armoring anti-PSMA-CAR-T cells with a TGFβRIIDN armoring molecule for treating castrate-resistant prostate cancer (NCT03089203).

In some aspects, the armoring molecule comprises a dominant-negative TGF-β receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the armoring molecule comprises the amino acid sequence set forth in SEQ ID NO: 105.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) an armoring molecule comprising the amino acid sequence set forth in SEQ ID NO: 105.

In some aspects, the armoring molecule comprises a dominant-negative TGF-β receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the armoring molecule comprises the amino acid sequence set forth in SEQ ID NO: 105.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising the amino acid sequence set forth in SEQ ID NO: 105.

In some aspects, the polynucleotide comprises (a) a nucleotide sequence encoding a CAR, wherein the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99; and (b) a nucleotide sequence encoding a TGFβRIIDN. In some aspects, the polynucleotide comprises (a) a nucleotide sequence encoding a CAR, wherein the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 108; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 108; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 108; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 109; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 109; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 109; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 110; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 110; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 110; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 111; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 111; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 111; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 112; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 112; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 112; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 113; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 113; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 113; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 114; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 114; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 114; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 115; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 115; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 115; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 116; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 116; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 116; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 118; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 118; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 118; and (b) an armoring molecule comprising the amino acid sequence set forth in SEQ ID NO: 105.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 119; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 119; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 119; and (b) an armoring molecule comprising the amino acid sequence set forth in SEQ ID NO: 105.

In some aspects, the polynucleotide comprises (a) a nucleotide sequence encoding a CAR comprising the nucleotide sequence set forth in SEQ ID NO: 101; and (b) a nucleotide sequence encoding an armoring molecule comprising the nucleotide sequence set forth in SEQ ID NO: 104.

In some aspects, the polynucleotide comprises (a) a nucleotide sequence encoding a CAR comprising the nucleotide sequence set forth in SEQ ID NO: 102; and (b) a nucleotide sequence encoding an armoring molecule comprising the nucleotide sequence set forth in SEQ ID NO: 104.

In some aspects, the polynucleotide comprises (a) a nucleotide sequence encoding a CAR comprising the nucleotide sequence set forth in SEQ ID NO: 103; and (b) a nucleotide sequence encoding an armoring molecule comprising the nucleotide sequence set forth in SEQ ID NO: 104.

In some aspects, the polynucleotide comprises (a) a nucleotide sequence encoding a CAR comprising the nucleotide sequence set forth in SEQ ID NO: 101; and (b) a nucleotide sequence encoding an armoring molecule comprising the amino acid sequence set forth in SEQ ID NO: 105.

In some aspects, the polynucleotide comprises (a) a nucleotide sequence encoding a CAR comprising the nucleotide sequence set forth in SEQ ID NO: 102; and (b) a nucleotide sequence encoding an armoring molecule comprising the amino acid sequence set forth in SEQ ID NO: 105.

In some aspects, the polynucleotide comprises (a) a nucleotide sequence encoding a CAR comprising the nucleotide sequence set forth in SEQ ID NO: 103; and (b) a nucleotide sequence encoding an armoring molecule comprising the amino acid sequence set forth in SEQ ID NO: 105.

In some aspects, the nucleotide encoding the CAR and the nucleotide encoding the armoring molecule are linked by a third nucleotide sequence, wherein the third nucleotide sequence encodes a cleavable peptide linker. In some aspects, the cleavable peptide linker comprises a T2A peptide. In some aspects, the cleavable peptide linker comprises SEQ ID NO: 126.

In some aspects, the polynucleotide comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 107. In some aspects, the polynucleotide comprises the nucleotide sequence set forth in SEQ ID NO: 107.

### II. Anti-STEAP2 Antibodies of the Present Disclosure

Some aspects of the present disclosure are directed to antibodies or antigen-binding portions thereof that specifically binds human STEAP2. In some aspects, the antibody or antigen-binding portion thereof comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3. In some aspects, the antibody or antigen-binding portion thereof comprises a VH-CDR3 comprising an amino acid sequence selected from SEQ ID NOs: 6, 16, 26, 36, 46, and 96. In some aspects, the antibody or antigen-binding portion thereof comprises a VH-CDR2 comprising an amino acid sequence selected from SEQ ID NOs: 5, 15, 25, 35, 45, and 95. In some aspects, the antibody or antigen-binding portion thereof comprises a VH-CDR1 comprising an amino acid sequence selected from SEQ ID NOs: 4, 14, 24, 34, and 44, and 94.

In some aspects, the antibody or antigen-binding portion thereof comprises a VL-CDR3 comprising an amino acid sequence selected from SEQ ID NOs: 3, 13, 23, 33, 43, and 93. In some aspects, the antibody or antigen-binding portion thereof comprises a VL-CDR2 comprising an amino acid sequence selected from SEQ ID NOs: 2, 12, 22, 32, 42, and 92. In some aspects, the antibody or antigen-binding portion thereof comprises a VL-CDR1 comprising an amino acid sequence selected from SEQ ID NOs: 1, 11, 21, 31, 41, and 91.

In some aspects, the antibody or antigen-binding portion thereof comprises a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the antibody or antigen-binding portion thereof comprises a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16.

In some aspects, the antibody or antigen-binding portion thereof comprises a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26.

In some aspects, the antibody or antigen-binding portion thereof comprises a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36.

In some aspects, the antibody or antigen-binding portion thereof comprises a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46.

In some aspects, the antibody or antigen-binding portion thereof comprises the VL-CDR1, VL-CDR2, and VL-CDR3 present in the VL region having the amino acid sequence set forth in SEQ ID NO: 58; and the VH-CDR1, VH-CDR2, and VH-CDR3 present in the VH region having the amino acid sequence set forth in SEQ ID NO: 57.

In some aspects, the antibody or antigen-binding portion thereof comprises the VL-CDR1, VL-CDR2, and VL-CDR3 present in the VL region having the amino acid sequence set forth in SEQ ID NO: 68; and the VH-CDR1, VH-CDR2, and VH-CDR3 present in the VH region having the amino acid sequence set forth in SEQ ID NO: 67.

In some aspects, the antibody or antigen-binding portion thereof comprises the VL-CDR1, VL-CDR2, and VL-CDR3 present in the VL region having the amino acid sequence set forth in SEQ ID NO: 78; and the VH-CDR1, VH-CDR2, and VH-CDR3 present in the VH region having the amino acid sequence set forth in SEQ ID NO: 77.

In some aspects, the antibody or antigen-binding portion thereof comprises the VL-CDR1, VL-CDR2, and VL-CDR3 present in the VL region having the amino acid sequence set forth in SEQ ID NO: 88; and the VH-CDR1, VH-CDR2, and VH-CDR3 present in the VH region having the amino acid sequence set forth in SEQ ID NO: 87.

In some aspects, the antibody or antigen-binding portion thereof comprises the VL-CDR1, VL-CDR2, and VL-CDR3 present in the VL region having the amino acid sequence set forth in SEQ ID NO: 98; and the VH-CDR1, VH-CDR2, and VH-CDR3 present in the VH region having the amino acid sequence set forth in SEQ ID NO: 97.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, and 97. In some aspects, the antibody or antigen-binding portion thereof comprises a VH comprising an amino acid sequence selected from SEQ ID NOs: 7, 17, 27, 37, 47, 57, 67, 77, 87, and 97.

In some aspects, the antibody or antigen-binding portion thereof comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, and 98. In some aspects, the antibody or antigen-binding portion thereof comprises a VL comprising an amino acid sequence selected from SEQ ID NOs: 8, 18, 28, 38, 48, 58, 68, 78, 88, and 98.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the antibody or antigen-binding portion thereof comprises a VH comprising the amino acid sequence set forth in SEQ ID NO: 7, and a VL comprising the amino acid sequence set forth in SEQ ID NO: 8.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the antibody or antigen-binding portion thereof comprises a VH comprising the amino acid sequence set forth in SEQ ID NO: 17, and a VL comprising the amino acid sequence set forth in SEQ ID NO: 18.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28. In some aspects, the antibody or antigen-binding portion thereof comprises a VH comprising the amino acid sequence set forth in SEQ ID NO: 27, and a VL comprising the amino acid sequence set forth in SEQ ID NO: 28.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38. In some aspects, the antibody or antigen-binding portion thereof comprises a VH comprising the amino acid sequence set forth in SEQ ID NO: 37, and a VL comprising the amino acid sequence set forth in SEQ ID NO: 38.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48. In some aspects, the antibody or antigen-binding portion thereof comprises a VH comprising the amino acid sequence set forth in SEQ ID NO: 47, and a VL comprising the amino acid sequence set forth in SEQ ID NO: 48.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58. In some aspects, the antibody or antigen-binding portion thereof comprises a VH comprising the amino acid sequence set forth in SEQ ID NO: 57, and a VL comprising the amino acid sequence set forth in SEQ ID NO: 58.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68. In some aspects, the antibody or antigen-binding portion thereof comprises a VH comprising the amino acid sequence set forth in SEQ ID NO: 67, and a VL comprising the amino acid sequence set forth in SEQ ID NO: 68.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78. In some aspects, the antibody or antigen-binding portion thereof comprises a VH comprising the amino acid sequence set forth in SEQ ID NO: 77, and a VL comprising the amino acid sequence set forth in SEQ ID NO: 78.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88. In some aspects, the antibody or antigen-binding portion thereof comprises a VH comprising the amino acid sequence set forth in SEQ ID NO: 87, and a VL comprising the amino acid sequence set forth in SEQ ID NO: 88.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the antibody or antigen-binding portion thereof comprises a VH comprising the amino acid sequence set forth in SEQ ID NO: 97, and a VL comprising the amino acid sequence set forth in SEQ ID NO: 98.

In some aspects, the antibody or antigen-binding portion thereof comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9. In some aspects, the antibody or antigen-binding portion thereof comprises the amino acid sequence set forth in SEQ ID NO: 9.

In some aspects, the antibody or antigen-binding portion thereof comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99. In some aspects, the antibody or antigen-binding portion thereof comprises the amino acid sequence set forth in SEQ ID NO: 99.

In some aspects, the antibody or antigen-binding portion thereof cross competes for binding to human STEAP2 with an antibody or antigen-binding portion thereof disclosed herein. In some aspects, the antibody or antigen-binding portion thereof binds the same epitope on human STEAP2 as an antibody or antigen-binding portion thereof disclosed herein. In some aspects, the antibody or antigen-binding portion thereof binds on overlapping epitope on human STEAP2 as an antibody or antigen-binding portion thereof disclosed herein.

### III. Cells of the Disclosure

Some aspects of the present disclosure are directed to cells comprising a polynucleotide or a polypeptide disclosed herein. Some aspects of the present disclosure are directed to a cell comprising (i) a polynucleotide encoding a chimeric antigen receptor (CAR) that binds human STEAP2. In some aspects, the cell further comprises (ii) a polynucleotide encoding an armoring molecule. In some aspects, the cell is an immune cell. In some aspects, the cell is selected from the group consisting of a T cell, a Natural Killer (NK) cell, a cytotoxic T lymphocyte (CTL), a regulatory T cell, a tumor infiltrating lymphocyte, and any combination thereof. In some aspects, the cell is a mammalian cell. In some aspects, the cell is a human cell.

The cell of the present disclosure can be obtained through any source. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain aspects, the cells collected by apheresis are washed to remove the plasma fraction, and placed in an appropriate buffer or media for subsequent processing. In some aspects, the cells are washed with PBS. As will be appreciated, a washing step can be used, such as by using a semiautomated flowthrough centrifuge, e.g., the COBE™ 2991 cell processor, the Baxter CYTOMATE™, or the like. In some aspects, the washed cells are resuspended in one or more biocompatible buffers, or other saline solution with or without buffer. In certain aspects, the undesired components of the apheresis sample are removed. Additional methods of isolating T cells for a T cell therapy are disclosed in U.S. Patent Publication No. 2013/0287748, which is herein incorporated by references in its entirety.

In certain aspects, T cells are isolated from PBMCs by lysing the red blood cells and depleting the monocytes, e.g., by using centrifugation through a PERCOLL™ gradient. In some aspects, a specific subpopulation of T cells, such as CD28+, CD4+, CD8+, CD45RA+, and CD45RO+ T cells is further isolated by positive or negative selection techniques known in the art. For example, enrichment of a T cell population by negative selection can be accomplished with a combination of antibodies directed to surface markers unique to the negatively selected cells. In some aspects, cell sorting and/or selection via negative magnetic immunoadherence or flow cytometry that uses a cocktail of monoclonal antibodies directed to cell surface markers present on the cells negatively selected can be used. For example, to enrich for CD4+ cells by negative selection, a monoclonal antibody cocktail typically includes antibodies to CD14, CD20, CD11b, CD16, HLA-DR, and CD8. In certain aspects, flow cytometry and cell sorting are used to isolate cell populations of interest for use in the present disclosure.

In some aspects, PBMCs are used directly for genetic modification with the immune cells (such as CARs) using methods as described herein. In certain aspects, after isolating the PBMCs, T lymphocytes are further isolated, and both cytotoxic and helper T lymphocytes are sorted into naive, memory, and effector T cell subpopulations either before or after genetic modification and/or expansion.

In some aspects, CD8+ cells are further sorted into naive, central memory, and effector cells by identifying cell surface antigens that are associated with each of these types of CD8+ cells. In some aspects, the expression of phenotypic markers of central memory T cells includes CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127 and positive for granzyme B and perforin. In certain aspects, CD4+ T cells are further sorted into subpopulations. For example, CD4+ T helper cells can be sorted into naive, central memory, and effector cells by identifying cell populations that have cell surface antigens.

In some aspects, the immune cells, e.g., T cells, are genetically modified following isolation using known methods, or the immune cells are activated and expanded (or differentiated in the case of progenitors) in vitro prior to being genetically modified. In another aspect, the immune cells, e.g., T cells, are genetically modified with the CARs described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other aspects, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In certain aspects, the T cells are obtained from a donor subject. In some aspects, the donor subject is human patient afflicted with a cancer or a tumor. In other aspects, the donor subject is a human patient not afflicted with a cancer or a tumor.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the amino acid sequence set forth in SEQ ID NO: 105.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising the amino acid sequence set forth in SEQ ID NO: 105.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) an armoring molecule comprising the amino acid sequence set forth in SEQ ID NO: 105.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the amino acid sequence set forth in SEQ ID NO: 105.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising the amino acid sequence set forth in SEQ ID NO: 105.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) an armoring molecule comprising the amino acid sequence set forth in SEQ ID NO: 105.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 104. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the nucleic acid sequence set forth in SEQ ID NO: 104.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 104. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16; and (b) an armoring molecule comprising the nucleic acid sequence set forth in SEQ ID NO: 104.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) an armoring molecule comprising a nucleic acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 104. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96; and (b) an armoring molecule comprising the nucleic acid sequence set forth in SEQ ID NO: 104.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising nucleic acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 104. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the nucleic acid sequence set forth in SEQ ID NO: 104.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 104. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18; and (b) an armoring molecule comprising the nucleic acid sequence set forth in SEQ ID NO: 104.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) an armoring molecule comprising a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 104. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98; and (b) an armoring molecule comprising the nucleic acid sequence set forth in SEQ ID NO: 104.

In some aspects, the cell comprises a polynucleotide comprising (a) a nucleotide sequence encoding a CAR comprising the nucleotide sequence set forth in SEQ ID NO: 101; and (b) a nucleotide sequence encoding an armoring molecule comprising the nucleotide sequence set forth in SEQ ID NO: 104.

In some aspects, the cell comprises a polynucleotide comprising (a) a nucleotide sequence encoding a CAR comprising the nucleotide sequence set forth in SEQ ID NO: 102; and (b) a nucleotide sequence encoding an armoring molecule comprising the nucleotide sequence set forth in SEQ ID NO: 104.

In some aspects, the cell comprises a polynucleotide comprising (a) a nucleotide sequence encoding a CAR comprising the nucleotide sequence set forth in SEQ ID NO: 103; and (b) a nucleotide sequence encoding an armoring molecule comprising the nucleotide sequence set forth in SEQ ID NO: 104.

In some aspects, the cell comprises a polynucleotide comprising (a) a nucleotide sequence encoding a CAR comprising the nucleotide sequence set forth in SEQ ID NO: 101; and (b) a nucleotide sequence encoding an armoring molecule comprising the amino acid sequence set forth in SEQ ID NO: 105.

In some aspects, the cell comprises a polynucleotide comprising (a) a nucleotide sequence encoding a CAR comprising the nucleotide sequence set forth in SEQ ID NO: 102; and (b) a nucleotide sequence encoding an armoring molecule comprising the amino acid sequence set forth in SEQ ID NO: 105.

In some aspects, the cell comprises a polynucleotide comprising (a) a nucleotide sequence encoding a CAR comprising the nucleotide sequence set forth in SEQ ID NO: 103; and (b) a nucleotide sequence encoding an armoring molecule comprising the amino acid sequence set forth in SEQ ID NO: 105.

In some aspects, the cell comprises a polynucleotide comprising a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 107. In some aspects, the polynucleotide comprises the nucleotide sequence set forth in SEQ ID NO: 107.

In some aspects, the cell comprises a polynucleotide comprising a nucleotide sequence encoding a polypeptide comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 106. In some aspects, the polypeptide comprises the amino acid sequence set forth in SEQ ID NO: 106.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 117. In some aspects, the polypeptide comprises the amino acid sequence set forth in SEQ ID NO: 117.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 120. In some aspects, the polypeptide comprises the amino acid sequence set forth in SEQ ID NO: 120.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 121. In some aspects, the polypeptide comprises the amino acid sequence set forth in SEQ ID NO: 121.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 122. In some aspects, the polypeptide comprises the amino acid sequence set forth in SEQ ID NO: 122.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 123. In some aspects, the polypeptide comprises the amino acid sequence set forth in SEQ ID NO: 123.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 124. In some aspects, the polypeptide comprises the amino acid sequence set forth in SEQ ID NO: 124.

In some aspects, the cell comprises a polynucleotide comprising a nucleotide sequence encoding an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 125. In some aspects, the polypeptide comprises the amino acid sequence set forth in SEQ ID NO: 125.

In some aspects, the cell comprises a CAR comprising an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the cell comprises a CAR comprising an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16.

In some aspects, the cell comprises a CAR comprising an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96.

In some aspects, the cell comprises a polypeptide that comprises (i) a CAR comprising an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6 and (ii) an amino acid sequence set forth in SEQ ID NO: 105.

In some aspects, the cell comprises a polypeptide that comprises (i) a CAR comprising an antigen-binding domain comprising a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16 and (ii) an amino acid sequence set forth in SEQ ID NO: 105.

In some aspects, the cell comprises a polypeptide that comprises (i) a CAR comprising an antigen-binding domain comprising a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96 and (ii) an amino acid sequence set forth in SEQ ID NO: 105.

In some aspects, the cell comprises a CAR comprising an amino acid set forth in SEQ ID NO: 10 and an armoring molecule comprising an amino acid set forth in SEQ ID NO: 105. In some aspects, the cell comprises a CAR comprising an amino acid set forth in SEQ ID NO: 108 and an armoring molecule comprising an amino acid set forth in SEQ ID NO: 105. In some aspects, the cell comprises a CAR comprising an amino acid set forth in SEQ ID NO: 109 and an armoring molecule comprising an amino acid set forth in SEQ ID NO: 105. In some aspects, the cell comprises a CAR comprising an amino acid set forth in SEQ ID NO: 110 and an armoring molecule comprising an amino acid set forth in SEQ ID NO: 105. In some aspects, the cell comprises a CAR comprising an amino acid set forth in SEQ ID NO: 111 and an armoring molecule comprising an amino acid set forth in SEQ ID NO: 105. In some aspects, the cell comprises a CAR comprising an amino acid set forth in SEQ ID NO: 112 and an armoring molecule comprising an amino acid set forth in SEQ ID NO: 105. In some aspects, the cell comprises a CAR comprising an amino acid set forth in SEQ ID NO: 113 and an armoring molecule comprising an amino acid set forth in SEQ ID NO: 105. In some aspects, the cell comprises a CAR comprising an amino acid set forth in SEQ ID NO: 114 and an armoring molecule comprising an amino acid set forth in SEQ ID NO: 105. In some aspects, the cell comprises a CAR comprising an amino acid set forth in SEQ ID NO: 115 and an armoring molecule comprising an amino acid set forth in SEQ ID NO: 105. In some aspects, the cell comprises a CAR comprising an amino acid set forth in SEQ ID NO: 116 and an armoring molecule comprising an amino acid set forth in SEQ ID NO: 105.

### IV. Vectors, Host Cells, and Pharmaceutical Compositions of the Disclosure

In some aspects, the polynucleotide of the present disclosure is present in a vector. As such, provided herein are vectors comprising a polynucleotide of the present disclosure. In some aspects, the present disclosure is directed to a vector or a set of vectors comprising a polynucleotide encoding a CAR, as described herein. In other aspects, the present disclosure is directed to a vector or a set of vectors comprising a polynucleotide encoding an antibody or an antigen binding molecule thereof that specifically binds to STEAP2, as disclosed herein.

In some aspects, the set of vectors comprises a first vector and a second vector, wherein the first vector comprises a nucleic acid sequence encoding a CAR disclosed herein, and the second vector comprises a nucleic acid sequence encoding an armoring molecule disclosed herein.

Any vector known in the art can be suitable for the present disclosure. In some aspects, the vector is a viral vector. In some aspects, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

In other aspects, provided herein are host cells comprising a polynucleotide or a vector of the present disclosure. In some aspects, the present disclosure is directed to host cells, e.g., in vitro cells, comprising a polynucleotide encoding a CAR or a TCR, as described herein. In some aspects, the present disclosure is directed to host cells, e.g., in vitro cells, comprising a polynucleotide encoding an antibody or an antigen binding molecule thereof that specifically binds to STEAP2, as disclosed herein. In other aspects, the present disclosure is directed to in vitro cells comprising a polypeptide encoded by a polynucleotide encoding a CAR—That specifically binds to STEAP2. In other aspects, the present disclosure is directed to cells, in vitro cells, comprising a polypeptide encoded by a polynucleotide encoding an antibody or an antigen binding molecule thereof that specifically binds to STEAP2, as disclosed herein.

Any cell may be used as a host cell for the polynucleotides, the vectors, or the polypeptides of the present disclosure. In some aspects, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some aspects, the cell is a human cell.

Other aspects of the present disclosure are directed to compositions comprising a polynucleotide described herein, a vector described herein, a polypeptide described herein, or cell described herein. In some aspects, the composition comprises a pharmaceutically acceptable carrier, diluent, solubilizer, emulsifier, preservative and/or adjuvant. In some aspects, the composition comprises an excipient. In one aspect, the composition comprises a polynucleotide encoding a CAR, wherein the CAR comprises an antigen binding molecule that specifically binds to STEAP2. In another aspect, the composition comprises a CAR encoded by a polynucleotide of the present disclosure, wherein the CAR comprises an antigen binding molecule that specifically binds to STEAP2. In another aspect, the composition comprises a T cell comprising a polynucleotide encoding a CAR, wherein the CAR comprises an antigen binding molecule that specifically binds to STEAP2. In another aspect, the composition comprises an antibody or an antigen binding molecule thereof that specifically binds STEAP2, as described herein. In another aspect, the composition comprises a cell (e.g., a T cell, e.g., a CAR-T cell) comprising a polynucleotide encoding CAR comprising an antigen binding domain that specifically binds STEAP2, as disclosed herein.

In other aspects, the composition is formulated for parenteral delivery, for inhalation, or for delivery through the digestive tract, such as orally. The preparation of such pharmaceutically acceptable compositions is within the ability of one skilled in the art. In certain aspects, buffers are used to maintain the composition at physiological pH or at a slightly lower pH, typically within a pH range of from about 5 to about 8. In certain aspects, when parenteral administration is contemplated, the composition is in the form of a pyrogen-free, parenterally acceptable aqueous solution comprising a desired antigen binding molecule to BCMA, with or without additional therapeutic agents, in a pharmaceutically acceptable vehicle. In certain aspects, the vehicle for parenteral injection is sterile distilled water in which an antigen binding molecule to BCMA, with or without at least one additional therapeutic agent, is formulated as a sterile, isotonic solution, properly preserved. In certain aspects, the preparation involves the formulation of the desired molecule with polymeric compounds (such as polylactic acid or polyglycolic acid), beads or liposomes, that provide for the controlled or sustained release of the product, which are then be delivered via a depot injection. In certain aspects, implantable drug delivery devices are used to introduce the desired molecule.

### VI. Methods of the Disclosure

Certain aspects of the present disclosure are directed to methods of treating a disease or condition in a subject in need thereof, comprising administering to the subject a composition disclosed herein. In some aspects, the disease or condition comprises a cancer. In some aspects, the cancer is prostate cancer. In some aspects, the cancer comprises a tumor derived from a prostate cancer (e.g., a tumor arising from the metastasis of a prostate cancer). In some aspects, the cancer (e.g., the prostate cancer) is locally progressed. In some aspects, the cancer (e.g., the prostate cancer) is metastatic. In some aspects, the cancer (e.g., the prostate cancer) is recurrent. In some aspects, the cancer (e.g., the prostate cancer) is relapsed.

The compositions disclosed herein, e.g. a T-cell comprising a polynucleotide encoding a CAR disclosed herein, can be used in combination with other anti-cancer therapies, including one or more additional immunotherapies. In some aspects, the compositions disclosed herein are administered concurrently with the additional anti-cancer agent. In some aspects, the compositions disclosed herein and the additional anti-cancer agent are administered sequentially (e.g., on the same day or on different days).

In some aspects, the additional anti-cancer agent comprises an antimetabolites (including, without limitation, folic acid antagonists, pyrimidine analogs, purine analogs and adenosine deaminase inhibitors). In some aspects, the additional anti-cancer agent comprises methotrexate, 5-fluorouracil, floxuridine, cytarabine, 6-mercaptopurine, 6-thioguanine, fludarabine phosphate, pentostatine, gemcitabine, and any combination thereof.

In some aspects, the additional anti-cancer agent comprises a taxane, paclitaxel (e.g., TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone Bi, [17]-dehydrodesoxyepothilone B, dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone BIO, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, a microtubuline stabilizing, and any combination thereof.

TABLE 1
Sequences.

| SEQ | | |
|---|---|---|
| ID | Description | Sequences |
| 1 | STEAP2 | RASQSVNSNLA |
| | VL CDR1 | |
| | (40A3) | |
| 2 | STEAP2 | GASTRAT |
| | VL CDR2 | |
| 3 | STEAP2 | QQYNNWPFT |
| | VL CDR3 | |
| 4 | STEAP2 | RNSAVWN |
| | VH CDR1 | |
| 5 | STEAP2 | RTYYRSKWYNDYAVSVKS |
| | VH CDR2 | |
| 6 | STEAP2 | GLLQNNFYYYMDV |
| | VH CDR3 | |
| 7 | STEAP2 | QVQLQQSGPGLVKPSQTLSLTCAISGDSVSRNSAVWNWIRQS |
| | VH | PSRGLEWLGRTYYRSKWYNDYAVSVKSRITINPDTSKNQFSL |
| | | QVNSVTPEDTAVYYCARGLLQNNFYYYMDVWGKGTTVTVSS |
| 8 | STEAP2 | EIVMTQSPATLSVSPGERATLSCRASQSVNSNLAWYQQKPGQ |
| | VL | APRLLIYGASTRATGIPARFSGSGSGTEFTLTISSLQSEDFA |
| | | VYYCQQYNNWPFTFGPGTKVDIK |
| 9 | STEAP2 | EIVMTQSPATLSVSPGERATLSCRASQSVNSNLAWYQQKPGQ |
| | scFv | APRLLIYGASTRATGIPARFSGSGSGTEFTLTISSLQSEDFA |
| | amino | VYYCQQYNNWPFTFGPGTKVDIKGGGGSGGGGSGGGGSGGGG |
| | acid | SQVQLQQSGPGLVKPSQTLSLTCAISGDSVSRNSAVWNWIRQ |
| | sequence | SPSRGLEWLGRTYYRSKWYNDYAVSVKSRITINPDTSKNQFS |
| | | LQVNSVTPEDTAVYYCARGLLQNNFYYYMDVWGKGTTVTVSS |
| 10 | STEAP2 | MLLLVTSLLLCELPHPAFLLIPEIVMTQSPATLSVSPGERAT |
| | BZ CAR | LSCRASQSVNSNLAWYQQKPGQAPRLLIYGASTRATGIPARF |
| | amino | SGSGSGTEFTLTISSLQSEDFAVYYCQQYNNWPFTFGPGTKV |
| | acid | DIKGGGGSGGGGSGGGGSGGGGSQVQLQQSGPGLVKPSQTLS |
| | sequence | LTCAISGDSVSRNSAVWNWIRQSPSRGLEWLGRTYYRSKWYN |
| | | DYAVSVKSRITINPDTSKNQFSLQVNSVTPEDTAVYYCARGL |
| | | LQNNFYYYMDVWGKGTTVTVSSGSESKYGPPCPPCPFWVLVV |
| | | VGGVLACYSLLVTVAFIIFWVKRGRKKLLYIFKQPFMRPVOT |
| | | TQEEDGCSCRFPEEEEGGCELRVKFSRSADAPAYQQGQNQLY |
| | | NELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 11 | STEAP2-3 | RASQSVSSNLA |
| | VL | |
| | CDR1 | |
| | (40A3GL-LO7) | |
| 12 | STEAP2-3 | GASTRAT |
| | VL CDR2 | |
| 13 | STEAP2-3 | QQYNNWPFT |
| | VL CDR3 | |
| 14 | STEAP2-3 | RNSAVWN |
| | VH CDR1 | |
| 15 | STEAP2-3 | RTYYRSKWYNDYAVSVKS |
| | VH CDR2 | |
| 16 | STEAP2-3 | GLLQNQFYYYMDV |
| | VH | |
| | CDR3 | |
| 17 | STEAP2-3 | QVQLQQSGPGLVKPSQTLSLTCAISGDSVSRNSAVWNWIRQS |
| | VH | PSRGLEWLGRTYYRSKWYNDYAVSVKSRITINPDTSKNQFSL |
| | | QLNSVTPEDTAVYYCARGLLQNQFYYYMDVWGKGTTVTVSS |
| 18 | STEAP2-3 | EIVMTQSPATLSVSPGERATLSCRASQSVSSNLAWYQQKPGQ |
| | VL | APRLLIYGASTRATGIPARFSGSGSGTEFTLTISSLQSEDFA |
| | | VYYCQQYNNWPFTFGPGTKVDIK |
| 21 | STEAP2-2 | RSSQSVVHSNGNTYLE |
| | VL | |
| | CDR1 (mm | |
| | 30D12) | |
| 22 | STEAP2-2 | KVSNRFS |
| | VL CDR2 | |
| 23 | STEAP2-2 | FQGSHVPYT |
| | VL CDR3 | |
| 24 | STEAP2-2 | SYGMS |
| | VH CDR1 | |
| 25 | STEAP2-2 | TISSGGSYTFYPDIMKG |
| | VH CDR2 | |
| 26 | STEAP2-2 | RGYGTIYTFSFDS |
| | VH CDR3 | |
| 27 | STEAP2-2 | EVQLVESGGDLVKPGGSLKLSCAASGFSFSSYGMSWVRQTPD |
| | VH | KRLEWVATISSGGSYTFYPDIMKGRFTISRDNAMNTLYLQMS |
| | | SLKSEDSAMYYCARRGYGTIYTFSFDSWGQGTTLTVSS |
| 28 | STEAP2-2 | DVLMTQTPLSLPVSLGDQASISCRSSQSVVHSNGNTYLEWYL |
| | VL | QKPGQSPKLLIYKVSNRFSGVPDRFSGSGSGTDFTLKISRVE |
| | | AEDLGVYYCFQGSHVPYTFGGGTKLEIK |
| 31 | STEAP2-2 | RSSQSVVHSNANTYLE |
| | Humanized1 | |
| | VL | |
| | CDR1 | |
| 32 | STEAP2-2 | KVSNRFS |
| | Humanized1 | |
| | VL | |
| | CDR2 | |
| 33 | STEAP2-2 | FQGSHVPYT |
| | Humanized1 | |
| | VL | |
| | CDR3 | |
| 34 | STEAP2-2 | SYGMS |
| | Humanized1 | |
| | VH | |
| | CDR1 | |
| 35 | STEAP2-2 | TISSGGSYTFYPDIMKG |
| | Humanized1 | |
| | VH | |
| | CDR2 | |
| 36 | STEAP2-2 | RGYGTIYTFSFDA |
| | Humanized1 | |
| | VH | |
| | CDR3 | |
| 37 | STEAP2-2 | EVQLVESGGGLVKPGGSLRLSCAASGFTFSSYGMSWVRQAPG |
| | Humanized1 | KRLEWVATISSGGSYTFYPDIMKGRFTISRDNSKNTLYLQMN |
| | VH | SLRAEDTAVYYCARRGYGTIYTFSFDAWGQGTTLTVSS |
| 38 | STEAP2-2 | DVVMTQSPLSLPVTLGQPASISCRSSQSVVHSNANTYLEWYL |
| | Humanized1 | QKPGQSPQLLIYKVSNRFSGVPDRFSGSGSGTDFTLKISRVE |
| | VL | AEDVGVYYCFQGSHVPYTFGQGTKLEIK |
| 41 | STEAP2-2 | RSSQSVVHSNANTYLE |
| | Humanized2 | |
| | VL | |
| | CDR1 | |
| 42 | STEAP2-2 | KVSNRFS |
| | Humanized2 | |
| | VL | |
| | CDR2 | |
| 43 | STEAP2-2 | FQGSHVPYT |
| | Humanized2 | |
| | VL | |
| | CDR3 | |
| 44 | STEAP2-2 | SYGMS |
| | Humanized2 | |
| | VH | |
| | CDR1 | |
| 45 | STEAP2-2 | TISSGGSYTFYPDIMKG |
| | Humanized2 | |
| | VH | |
| | CDR2 | |
| 46 | STEAP2-2 | RGYGTIYTFSFDA |
| | Humanized2 | |
| | VH | |
| | CDR3 | |
| 47 | STEAP2-2 | EVQLLESGGGLVQPGGSLRLSCAASGFTFSSYGMSWVRQAPG |
| | Humanized2 | KRLEWVSTISSGGSYTFYPDIMKGRFTISRDNSKNTLYLQMN |
| | VH | SLRAEDTAVYYCARRGYGTIYTFSFDAWGQGTTLTVSS |
| 48 | STEAP2-2 | DVVMTQSPLSLPVTLGQPASISCRSSQSVVHSNANTYLEWYL |
| | Humanized | QKPGQSPQLLIYKVSNRFSGVPDRFSGSGSGTDFTLKISRVE |
| | VL | AEDVGVYYCFQGSHVPYTFGQGTKLEIK |
| 57 | STEAP2-4 | EVQLVESGGGLVQPGGSLRLSCAASGFTFSSFAMTWVRQAPG |
| | VH | KGLEWVSVITYSGGRTYYADSVKGRFTISRDNSKNTLYLQMN |
| | (40A1) | SLRAEDTAVYFCAKDRIAAVGPFDYWGQGTLVTVSS |
| 58 | STEAP2-4 | DIQLTQSPSFLSASVGDRVTITCRASQGISVYLAWYQQEPGK |
| | VL | APKLLIYAASTLQSGVPSRFSGSGSGTEFTLTISSLQPEDFA |
| | | TYYCQQLNSYPRTFGQGTKVEIK |
| 67 | STEAP2-5 | QVQLVQSGAEVKKPGASVKVSCKASGYTFTSYGISWVRQAPG |
| | VH | QGLEWMGWISGYTGNTNYAQKLQGRVTMTADTSTSTAYMELR |
| | (34C1) | SLRSDDTAVYYCARGGSYFDYWGQGTLVTVSS |
| 68 | STEAP2-5 | DIQMTQSPSTLSASVGDRVTITCRASQSISRWLAWYQQKPGK |
| | VL | APKLLIYKASSLESGVPSRFSGSGSGTEFTLTISSLQPDDFA |
| | | TYYCQQFNSFSPITFGQGTRLEIK |
| 77 | STEAP2-6 | QVQLQQPGAELVKPGASVKLSCKASGYTFTSYWMEWVKQRPG |
| | VH | QGLEWIGMIHPNSGITNYNERFKNKATLTVDKSSSTAYMQLS |
| | (6E10) | SLTSEDSAVYYCARDHYYILAYWGQGTLVTVSA |
| 78 | STEAP2-6 | DVLMTQTPLSLPVSLGDQASISCRSSQSVVHSNGNTYLEWYL |
| | VL | QKPGQSPKLLIYKVSNRFSGVPDRFSGSGSGTDFTLKISRVE |
| | | AEDLGVYYCFQGSHVPYTFGGGTKLEIK |
| 87 | STEAP2-7 | QVQLQQPGADLVKPGASVKMSCKASGHTFTNYWVTWVKQRPG |
| | VH | QGLEWIGNFYPGSGIIKYNENFRSKATLTVDISSSTAYMQLS |
| | (22F3) | SLTSEDSAVYYCARSKLGDSFYFDYWGQGTTLTVSS |
| 88 | STEAP2-7 | DVVMTQTPLSLPVSLGNQASISCRSSQSLVHSNGNTYLHWYL |
| | VL | QKPGQSPKLLIYKVSNRFSGVPDRFSGSGSGTDFTLKISRVE |
| | | AEDLGVYFCSQSTHVPLTFGAGTKLEIK |
| 91 | STEAP2-8 | RASQSVASNLA |
| | 40A3GL- | |
| | L014) VL | |
| | CDR1 | |
| 92 | STEAP2-8 | GASTRAT |
| | VL CDR2 | |
| 93 | STEAP2-8 | QQYNNWPFT |
| | VL CDR3 | |
| 94 | STEAP2-8 | RNSAVWN |
| | VH CDR1 | |
| 95 | STEAP2-8 | RTYYRSKWYNDYAPSVKS |
| | VH CDR2 | |
| 96 | STEAP2-8 | GLRQNQFYYYMDV |
| | VH CDR3 | |
| 97 | STEAP2-8 | QVQLQQSGPGLVKPSQTLSLTCAISGDSVSRNSAVWNWIRQS |
| | VH | PSRGLEWLGRTYYRSKWYNDYAPSVKSRITINPDTSKNQFSL |
| | | QLNSVTPEDTAVYYCARGLRQNQFYYYMDVWGKGTTVTVSS |
| 98 | STEAP2-8 | EIVMTQSPATLSVSPGERATLSCRASQSVASNLAWYQQKPGQ |
| | VL | APRLLIYGASTRATGIPARFSGSGSGTEFTLTISSLQSEDFA |
| | | VYYCQQYNNWPFTFGPGTKVDIK |
| 99 | STEAP2-8 | EIVMTQSPATLSVSPGERATLSCRASQSVASNLAWYQQKPGQ |
| | scFv | APRLLIYGASTRATGIPARFSGSGSGTEFTLTISSLQSEDFA |
| | | VYYCQQYNNWPFTFGPGTKVDIKGGGGSGGGGSGGGGSGGGG |
| | | SQVQLQQSGPGLVKPSQTLSLTCAISGDSVSRNSAVWNWIRQ |
| | | SPSRGLEWLGRTYYRSKWYNDYAPSVKSRITINPDTSKNQFS |
| | | LQLNSVTPEDTAVYYCARGLRQNQFYYYMDVWGKGTTVTVSS |
| 101 | STEAP2 | GAGATTGTGATGACCCAGAGCCCTGCAACTCTGAGCGTGTCA |
| | scFv | CCCGGAGAAAGGGCCACTCTGTCGTGTCGAGCATCGCAGTCC |
| | nucleic | GTGAACTCCAATCTCGCCTGGTACCAGCAGAAGCCTGGGCAG |
| | acid | GCCCCGAGGCTGCTCATCTACGGTGCCTCCACGAGAGCCACG |
| | sequence | GGAATTCCAGCGCGCTTTAGCGGATCCGGCTCGGGAACCGAG |
| | | TTCACCCTTACCATCTCATCGCTGCAATCCGAAGATTTCGCC |
| | | GTGTATTACTGTCAACAGTACAACAACTGGCCGTTCACCTTT |
| | | GGCCCGGGAACTAAGGTCGACATCAAGGGCGGCGGGGGCTCT |
| | | GGGGGTGGCGGAAGCGGCGGCGGCGGATCCGGTGGCGGCGGA |
| | | AGCCAAGTGCAGCTGCAGCAGTCCGGACCCGGACTCGTGAAG |
| | | CCGTCCCAGACTCTGTCCCTGACTTGCGCGATTTCCGGCGAT |
| | | TCCGTGTCCCGCAACTCCGCTGTGTGGAACTGGATCCGGCAG |
| | | TCGCCTTCGAGAGGACTGGAGTGGCTGGGACGGACCTACTAC |
| | | CGCTCAAAATGGTATAACGACTATGCTGTGTCCGTCAAGAGC |
| | | CGCATCACCATTAACCCCGATACCTCCAAGAACCAGTTCAGT |
| | | CTGCAAGTCAACAGCGTGACTCCTGAGGACACCGCCGTGTAC |
| | | TACTGCGCCCGGGGTCTGCTGCAAAACAACTTCTACTACTAC |
| | | ATGGACGTCTGGGGAAAGGGAACTACTGTGACCGTGTCCTCC |
| 102 | STEAP2 | ATGCTGCTCCTTGTCACATCACTGCTGCTCTGCGAACTGCCC |
| | BZ CAR | CACCCTGCATTCCTCCTGATCCCCGAGATTGTGATGACCCAG |
| | nucleic | AGCCCTGCAACTCTGAGCGTGTCACCCGGAGAAAGGGCCACT |
| | acid | CTGTCGTGTCGAGCATCGCAGTCCGTGAACTCCAATCTCGCC |
| | sequence | TGGTACCAGCAGAAGCCTGGGCAGGCCCCGAGGCTGCTCATC |
| | | TACGGTGCCTCCACGAGAGCCACGGGAATTCCAGCGCGCTTT |
| | | AGCGGATCCGGCTCGGGAACCGAGTTCACCCTTACCATCTCA |
| | | TCGCTGCAATCCGAAGATTTCGCCGTGTATTACTGTCAACAG |
| | | TACAACAACTGGCCGTTCACCTTTGGCCCGGGAACTAAGGTC |
| | | GACATCAAGGGCGGCGGGGGCTCTGGGGGTGGCGGAAGCGGC |
| | | GGCGGCGGATCCGGTGGCGGCGGAAGCCAAGTGCAGCTGCAG |
| | | CAGTCCGGACCCGGACTCGTGAAGCCGTCCCAGACTCTGTCC |
| | | CTGACTTGCGCGATTTCCGGCGATTCCGTGTCCCGCAACTCC |
| | | GCTGTGTGGAACTGGATCCGGCAGTCGCCTTCGAGAGGACTG |
| | | GAGTGGCTGGGACGGACCTACTACCGCTCAAAATGGTATAAC |
| | | GACTATGCTGTGTCCGTCAAGAGCCGCATCACCATTAACCCC |
| | | GATACCTCCAAGAACCAGTTCAGTCTGCAAGTCAACAGCGTG |
| | | ACTCCTGAGGACACCGCCGTGTACTACTGCGCCCGGGGTCTG |
| | | CTGCAAAACAACTTCTACTACTACATGGACGTCTGGGGAAAG |
| | | GGAACTACTGTGACCGTGTCCTCCGGCTCCGAATCAAAATAC |
| | | GGTCCGCCATGCCCACCGTGCCCCTTCTGGGTGCTCGTGGTC |
| | | GTCGGAGGGGTTCTGGCCTGCTACTCCCTGCTGGTCACCGTG |
| | | GCGTTTATCATCTTCTGGGTGAAGCGGGGAAGGAAGAAGCTA |
| | | CTGTACATTTTCAAGCAGCCTTTCATGCGGCCTGTGCAGACC |
| | | ACCCAGGAAGAGGACGGCTGTTCCTGCCGGTTCCCCGAGGAA |
| | | GAGGAAGGGGGTTGCGAGCTGCGCGTGAAGTTCAGCAGGAGC |
| | | GCTGATGCCCCAGCGTACCAACAGGGGCAAAACCAGTTGTAC |
| | | AACGAACTGAACCTTGGTCGGCGCGAAGAGTACGACGTGCTT |
| | | GACAAGCGCCGCGGCAGAGATCCCGAGATGGGTGGAAAGCCG |
| | | CGGCGGAAGAATCCGCAGGAAGGGCTCTACAACGAGCTCCAG |
| | | AAGGACAAGATGGCCGAAGCCTACAGCGAAATCGGGATGAAG |
| | | GGCGAAAGACGCCGGGGAAAAGGACACGACGGACTGTACCAG |
| | | GGGTTGTCGACCGCGACCAAGGACACCTACGACGCCCTGCAT |
| | | ATGCAAGCCTTGCCGCCGAGATGA |
| 103 | STEAP2-2 | GATGTTTTGATGACCCAAACTCCTCTCTCCCTGCCTGTCAGT |
| | scFv | CTTGGAGATCAAGCCTCCATCTCTTGCAGATCTAGTCAGAGT |
| | nucleic | GTTGTACATAGTAATGGAAACACCTATTTAGAATGGTACCTG |
| | acid | CAGAAACCAGGCCAGTCTCCAAAGCTCCTGATCTACAAAGTT |
| | sequence | TCCAACCGATTTTCTGGGGTCCCAGACAGGTTCAGTGGCAGT |
| | | GGATCAGGGACAGATTTCACACTCAAGATCAGCAGAGTGGAG |
| | | GCTGAGGATCTGGGAGTTTATTACTGCTTTCAAGGTTCACAT |
| | | GTTCCGTACACGTTCGGAGGGGGGACCAAGCTGGAAATAAAA |
| | | GGAGGCGGAGGATCTGGTGGTGGTGGATCTGGCGGCGGAGGA |
| | | AGTGGTGGCGGAGGCTCTGAGGTGCAGCTGGTGGAGTCTGGG |
| | | GGAGACTTAGTGAAGCCTGGAGGGTCCCTGAAACTCTCCTGT |
| | | GCCGCCTCTGGATTCTCTTTCTCCTCTTATGGCATGTCTTGG |
| | | GTTCGCCAGACTCCAGACAAGAGGCTGGAATGGGTCGCAACC |
| | | ATTAGTAGTGGTGGTAGTTACACCTTCTATCCCGACATTATG |
| | | AAGGGGCGATTCACCATCTCCAGAGACAATGCCATGAACACC |
| | | CTGTACCTGCAAATGAGCAGTCTGAAGTCTGAGGACTCAGCC |
| | | ATGTATTACTGTGCAAGACGGGGCTACGGTACTATCTACACG |
| | | TTTTCCTTTGACTCCTGGGGCCAAGGCACCACTCTCACAGTC |
| | | TCCAGC |
| 104 | Dominant- | ATGGGACGCGGGCTGCTTCGAGGACTCTGGCCACTTCATATC |
| | negative | GTGTTGTGGACTCGCATCGCTTCAACCATTCCGCCGCACGTG |
| | TGF-β | CAGAAGTCCGTGAACAATGACATGATCGTGACCGACAACAAC |
| | receptor | GGTGCAGTGAAGTTCCCACAGCTGTGCAAGTTCTGCGATGTC |
| | type 2 | AGATTCAGCACTTGCGACAACCAGAAGTCCTGCATGTCAAAC |
| | nucleic | TGCTCCATCACCTCCATCTGCGAGAAGCCTCAAGAGGTCTGC |
| | acid | GTGGCCGTGTGGCGGAAGAACGACGAGAACATCACCCTGGAA |
| | sequence | ACCGTGTGCCACGATCCGAAGCTGCCTTATCACGACTTCATT |
| | | CTGGAAGATGCCGCCTCGCCCAAGTGTATCATGAAAGAAAAG |
| | | AAAAAGCCCGGAGAAACGTTCTTCATGTGCTCGTGTAGCTCC |
| | | GACGAGTGCAACGACAACATTATCTTTAGCGAAGAGTACAAC |
| | | ACTTCCAACCCTGACCTCCTGCTCGTGATTTTTCAAGTCACC |
| | | GGCATTTCCCTGCTGCCCCCGCTGGGCGTGGCGATCTCGGTG |
| | | ATCATTATCTTCTACTGTTACCGGGTCAATAGGCAG |
| 105 | Dominant- | MGRGLLRGLWPLHIVLWTRIASTIPPHVQKSVNNDMIVTDNN |
| | negative | GAVKFPQLCKFCDVRFSTCDNQKSCMSNCSITSICEKPQEVC |
| | TGF-β | VAVWRKNDENITLETVCHDPKLPYHDFILEDAASPKCIMKEK |
| | receptor | KKPGETFFMCSCSSDECNDNIIESEEYNTSNPDLLLVIFQVT |
| | type 2 | GISLLPPLGVAISVIIIFYCYRVNRQ |
| | amino | |
| | acid | |
| | sequence | |
| 106 | STEAP2 | MLLLVTSLLLCELPHPAFLLIPEIVMTQSPATLSVSPGERAT |
| | BZ CAR- | LSCRASQSVNSNLAWYQQKPGQAPRLLIYGASTRATGIPARF |
| | T2A- | SGSGSGTEFTLTISSLQSEDFAVYYCQQYNNWPFTFGPGTKV |
| | Dominant- | DIKGGGGSGGGGSGGGGSGGGGSQVQLQQSGPGLVKPSQTLS |
| | negative | LTCAISGDSVSRNSAVWNWIRQSPSRGLEWLGRTYYRSKWYN |
| | TGF-β | DYAVSVKSRITINPDTSKNQFSLQVNSVTPEDTAVYYCARGL |
| | receptor | LQNNFYYYMDVWGKGTTVTVSSGSESKYGPPCPPCPFWVLVV |
| | type 2 | VGGVLACYSLLVTVAFIIFWVKRGRKKLLYIFKQPFMRPVQT |
| | amino | TQEEDGCSCRFPEEEEGGCELRVKFSRSADAPAYQQGQNQLY |
| | acid | NELNLGRREEYDVLDKRRGRDPEMGGKPRRKNPQEGLYNELQ |
| | sequence | KDKMAEAYSEIGMKGERRRGKGHDGLYQGLSTATKDTYDALH |
| | | MQALPPRGSGEGRGSLLTCGDVEENPGPMGRGLLRGLWPLHI |
| | | VLWTRIASTIPPHVQKSVNNDMIVTDNNGAVKFPQLCKFCDV |
| | | RFSTCDNQKSCMSNCSITSICEKPQEVCVAVWRKNDENITLE |
| | | TVCHDPKLPYHDFILEDAASPKCIMKEKKKPGETFFMCSCSS |
| | | DECNDNIIFSEEYNTSNPDLLLVIFQVTGISLLPPLGVAISV |
| | | IIIFYCYRVNRQ |
| 107 | STEAP2 | ATGCTGCTCCTTGTCACATCACTGCTGCTCTGCGAACTGCCC |
| | BZ CAR- | CACCCTGCATTCCTCCTGATCCCCGAGATTGTGATGACCCAG |
| | T2A- | AGCCCTGCAACTCTGAGCGTGTCACCCGGAGAAAGGGCCACT |
| | Dominant- | CTGTCGTGTCGAGCATCGCAGTCCGTGAACTCCAATCTCGCC |
| | negative | TGGTACCAGCAGAAGCCTGGGCAGGCCCCGAGGCTGCTCATC |
| | TGF-β | TACGGTGCCTCCACGAGAGCCACGGGAATTCCAGCGCGCTTT |
| | receptor | AGCGGATCCGGCTCGGGAACCGAGTTCACCCTTACCATCTCA |
| | type 2 | TCGCTGCAATCCGAAGATTTCGCCGTGTATTACTGTCAACAG |
| | nucleic | TACAACAACTGGCCGTTCACCTTTGGCCCGGGAACTAAGGTC |
| | acid | GACATCAAGGGCGGCGGGGGCTCTGGGGGTGGCGGAAGCGGC |
| | sequence | GGCGGCGGATCCGGTGGCGGCGGAAGCCAAGTGCAGCTGCAG |
| | | CAGTCCGGACCCGGACTCGTGAAGCCGTCCCAGACTCTGTCC |
| | | CTGACTTGCGCGATTTCCGGCGATTCCGTGTCCCGCAACTCC |
| | | GCTGTGTGGAACTGGATCCGGCAGTCGCCTTCGAGAGGACTG |
| | | GAGTGGCTGGGACGGACCTACTACCGCTCAAAATGGTATAAC |
| | | GACTATGCTGTGTCCGTCAAGAGCCGCATCACCATTAACCCC |
| | | GATACCTCCAAGAACGAGTTCAGTCTGCAAGTCAACAGCGTG |
| | | ACTCCTGAGGACACCGCCGTGTACTACTGCGCCCGGGGTCTG |
| | | CTGCAAAACAACTTCTACTACTACATGGACGTCTGGGGAAAG |
| | | GGAACTACTGTGACCGTGTCCTCCGGCTCCGAATCAAAATAC |
| | | GGTCCGCCATGCCCACCGTGCCCCTTCTGGGTGCTCGTGGTC |
| | | GTCGGAGGGGTTCTGGCCTGCTACTCCCTGCTGGTCACCGTG |
| | | GCGTTTATCATCTTCTGGGTGAAGCGGGGAAGGAAGAAGCTA |
| | | CTGTACATTTTCAAGCAGCCTTTCATGCGGCCTGTGCAGACC |
| | | ACCCAGGAAGAGGACGGCTGTTCCTGCCGGTTCCCCGAGGAA |
| | | GAGGAAGGGGGTTGCGAGCTGCGCGTGAAGTTCAGCAGGAGC |
| | | GCTGATGCCCCAGCGTACCAACAGGGGCAAAACCAGTTGTAC |
| | | AACGAACTGAACCTTGGTCGGCGCGAAGAGTACGACGTGCTT |
| | | GACAAGCGCCGCGGCAGAGATCCCGAGATGGGTGGAAAGCCG |
| | | CGGCGGAAGAATCCGCAGGAAGGGCTCTACAACGAGCTCCAG |
| | | AAGGACAAGATGGCCGAAGCCTACAGCGAAATCGGGATGAAG |
| | | GGCGAAAGACGCCGGGGAAAAGGACACGACGGACTGTACCAG |
| | | GGGTTGTCGACCGCGACCAAGGACACCTACGACGCCCTGCAT |
| | | ATGCAAGCCTTGCCGCCGAGAGGATCCGGAGAGGGGAGGGGA |
| | | AGCCTCCTCACTTGCGGCGATGTGGAGGAAAACCCGGGTCCT |
| | | ATGGGACGCGGGCTGCTTCGAGGACTCTGGCCACTTCATATC |
| | | GTGTTGTGGACTCGCATCGCTTCAACCATTCCGCCGCACGTG |
| | | CAGAAGTCCGTGAACAATGACATGATCGTGACCGACAACAAC |
| | | GGTGCAGTGAAGTTCCCACAGCTGTGCAAGTTCTGCGATGTC |
| | | AGATTCAGCACTTGCGACAACCAGAAGTCCTGCATGTCAAAC |
| | | TGCTCCATCACCTCCATCTGCGAGAAGCCTCAAGAGGTCTGC |
| | | GTGGCCGTGTGGCGGAAGAACGACGAGAACATCACCCTGGAA |
| | | ACCGTGTGCCACGATCCGAAGCTGCCTTATCACGACTTCATT |
| | | CTGGAAGATGCCGCCTCGCCCAAGTGTATCATGAAAGAAAAG |
| | | AAAAAGCCCGGAGAAACGTTCTTCATGTGCTCGTGTAGCTCC |
| | | GACGAGTGCAACGACAACATTATCTTTAGCGAAGAGTACAAC |
| | | ACTTCCAACCCTGACCTCCTGCTCGTGATTTTTCAAGTCACC |
| | | GGCATTTCCCTGCTGCCCCCGCTGGGCGTGGCGATCTCGGTG |
| | | ATCATTATCTTCTACTGTTACCGGGTCAATAGGCAG |
| 108 | STEAP2-2 | MLLLVTSLLLCELPHPAFLLIPDVLMTQTPLSLPVSLGDQAS |
| | BZ CAR | ISCRSSQSVVHSNGNTYLEWYLQKPGQSPKLLIYKVSNRFSG |
| | amino | VPDRFSGSGSGTDFTLKISRVEAEDLGVYYCFQGSHVPYTFG |
| | acid | GGTKLEIKGGGGSGGGGSGGGGSGGGGSEVQLVESGGDLVKP |
| | sequence | GGSLKLSCAASGFSFSSYGMSWVRQTPDKRLEWVATISSGGS |
| | | YTFYPDIMKGRFTISRDNAMNTLYLQMSSLKSEDSAMYYCAR |
| | | RGYGTIYTFSFDSWGQGTTLTVSSGSESKYGPPCPPCPFWVL |
| | | VVVGGVLACYSLLVTVAFIIFWVKRGRKKLLYIFKQPFMRPV |
| | | QTTQEEDGCSCRFPEEEEGGCELRVKFSRSADAPAYQQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYDA |
| | | LHMQALPPR |
| 109 | STEAP2-2 | MLLLVTSLLLCELPHPAFLLIPDVVMTQSPLSLPVTLGQPAS |
| | Humanized1 | ISCRSSQSVVHSNANTYLEWYLQKPGQSPQLLIYKVSNRFSG |
| | BZ | VPDRFSGSGSGTDFTLKISRVEAEDVGVYYCFQGSHVPYTFG |
| | CAR | QGTKLEIKGGGGSGGGGSGGGGSGGGGSEVQLVESGGGLVKP |
| | amino | GGSLRLSCAASGFTFSSYGMSWVRQAPGKRLEWVATISSGGS |
| | acid | YTFYPDIMKGRFTISRDNSKNTLYLQMNSLRAEDTAVYYCAR |
| | sequence | RGYGTIYTFSFDAWGQGTTLTVSSGSESKYGPPCPPCPFWVL |
| | | VVVGGVLACYSLLVTVAFIIFWVKRGRKKLLYIFKQPFMRPV |
| | | QTTQEEDGCSCRFPEEEEGGCELRVKFSRSADAPAYQQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYDA |
| | | LHMQALPPR |
| 110 | STEAP2-2 | MLLLVTSLLLCELPHPAFLLIPDVVMTQSPLSLPVTLGQPAS |
| | Humanized2 | ISCRSSQSVVHSNANTYLEWYLQKPGQSPQLLIYKVSNRFSG |
| | BZ | VPDRFSGSGSGTDFTLKISRVEAEDVGVYYCFQGSHVPYTFG |
| | CAR | QGTKLEIKGGGGSGGGGSGGGGSGGGGSEVQLLESGGGLVQP |
| | amino | GGSLRLSCAASGFTFSSYGMSWVRQAPGKRLEWVSTISSGGS |
| | acid | YTFYPDIMKGRFTISRDNSKNTLYLQMNSLRAEDTAVYYCAR |
| | sequence | RGYGTIYTFSFDAWGQGTTLTVSSGSESKYGPPCPPCPFWVL |
| | | VVVGGVLACYSLLVTVAFIIFWVKRGRKKLLYIFKQPFMRPV |
| | | QTTQEEDGCSCRFPEEEEGGCELRVKFSRSADAPAYQQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYDA |
| | | LHMQALPPR |
| 111 | STEAP2-3 | MLLLVTSLLLCELPHPAFLLIPEIVMTQSPATLSVSPGERAT |
| | BZ CAR | LSCRASQSVSSNLAWYQQKPGQAPRLLIYGASTRATGIPARF |
| | amino | SGSGSGTEFTLTISSLQSEDFAVYYCQQYNNWPFTFGPGTKV |
| | acid | DIKGGGGSGGGGSGGGGSGGGGSQVQLQQSGPGLVKPSQTLS |
| | sequence | LTCAISGDSVSRNSAVWNWIRQSPSRGLEWLGRTYYRSKWYN |
| | | DYAVSVKSRITINPDTSKNQFSLQLNSVTPEDTAVYYCARGL |
| | | LQNQFYYYMDVWGKGTTVTVSSGSESKYGPPCPPCPFWVLVV |
| | | VGGVLACYSLLVTVAFIIFWVKRGRKKLLYIFKQPFMRPVQT |
| | | TQEEDGCSCRFPEEEEGGCELRVKFSRSADAPAYQQGQNQLY |
| | | NELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 112 | STEAP2-4 | MLLLVTSLLLCELPHPAFLLIPDIQLTQSPSFLSASVGDRVT |
| | BZ CAR | ITCRASQGISVYLAWYQQEPGKAPKLLIYAASTLQSGVPSRF |
| | amino | SGSGSGTEFTLTISSLQPEDFATYYCQQLNSYPRTFGQGTKV |
| | acid | EIKGGGGSGGGGSGGGGSGGGGSEVQLVESGGGLVQPGGSLR |
| | sequence | LSCAASGFTFSSFAMTWVRQAPGKGLEWVSVITYSGGRTYYA |
| | | DSVKGRFTISRDNSKNTLYLQMNSLRAEDTAVYFCAKDRIAA |
| | | VGPFDYWGQGTLVTVSSGSESKYGPPCPPCPFWVLVVVGGVL |
| | | ACYSLLVTVAFIIFWVKRGRKKLLYIFKQPFMRPVQTTQEED |
| | | GCSCRFPEEEEGGCELRVKFSRSADAPAYQQGQNQLYNELNL |
| | | GRREEYDVLDKRRGRDPEMGGKPRRKNPQEGLYNELQKDKMA |
| | | EAYSEIGMKGERRRGKGHDGLYQGLSTATKDTYDALHMQALP |
| | | PR |
| 113 | STEAP2-5 | MLLLVTSLLLCELPHPAFLLIPDIQMTQSPSTLSASVGDRVT |
| | BZ CAR | ITCRASQSISRWLAWYQQKPGKAPKLLIYKASSLESGVPSRF |
| | amino | SGSGSGTEFTLTISSLQPDDFATYYCQQFNSFSPITFGQGTR |
| | acid | LEIKGGGGSGGGGSGGGGSGGGGSQVQLVQSGAEVKKPGASV |
| | sequence | KVSCKASGYTFTSYGISWVRQAPGQGLEWMGWISGYTGNTNY |
| | | AQKLQGRVTMTADTSTSTAYMELRSLRSDDTAVYYCARGGSY |
| | | FDYWGQGTLVTVSSGSESKYGPPCPPCPFWVLVVVGGVLACY |
| | | SLLVTVAFIIFWVKRGRKKLLYIFKQPFMRPVQTTQEEDGCS |
| | | CRFPEEEEGGCELRVKFSRSADAPAYQQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| 114 | STEAP2-6 | MLLLVTSLLLCELPHPAFLLIPDVLMTQTPLSLPVSLGDQAS |
| | BZ CAR | ISCRSSQSVVHSNGNTYLEWYLQKPGQSPKLLIYKVSNRFSG |
| | amino | VPDRFSGSGSGTDFTLKISRVEAEDLGVYYCFQGSHVPYTFG |
| | acid | GGTKLEIKGGGGSGGGGSGGGGSGGGGSQVQLQQPGAELVKP |
| | sequence | GASVKLSCKASGYTFTSYWMEWVKQRPGQGLEWIGMIHPNSG |
| | | ITNYNERFKNKATLTVDKSSSTAYMQLSSLTSEDSAVYYCAR |
| | | DHYYILAYWGQGTLVTVSAGSESKYGPPCPPCPFWVLVVVGG |
| | | VLACYSLLVTVAFIIFWVKRGRKKLLYIFKQPFMRPVQTTQE |
| | | EDGCSCRFPEEEEGGCELRVKFSRSADAPAYQQGQNQLYNEL |
| | | NLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |
| 115 | STEAP2-7 | MLLLVTSLLLCELPHPAFLLIPDVVMTQTPLSLPVSLGNQAS |
| | BZ CAR | ISCRSSQSLVHSNGNTYLHWYLQKPGQSPKLLIYKVSNRFSG |
| | amino | VPDRFSGSGSGTDFTLKISRVEAEDLGVYFCSQSTHVPLTFG |
| | acid | AGTKLEIKGGGGSGGGGSGGGGSGGGGSQVQLQQPGADLVKP |
| | sequence | GASVKMSCKASGHTFTNYWVTWVKQRPGQGLEWIGNFYPGSG |
| | | IIKYNENFRSKATLTVDISSSTAYMQLSSLTSEDSAVYYCAR |
| | | SKLGDSFYFDYWGQGTTLTVSSGSESKYGPPCPPCPFWVLVV |
| | | VGGVLACYSLLVTVAFIIFWVKRGRKKLLYIFKQPFMRPVQT |
| | | TQEEDGCSCRFPEEEEGGCELRVKFSRSADAPAYQQGQNQLY |
| | | NELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 116 | STEAP2-8 | MLLLVTSLLLCELPHPAFLLIPEIVMTQSPATLSVSPGERAT |
| | BZ CAR | LSCRASQSVASNLAWYQQKPGQAPRLLIYGASTRATGIPARF |
| | amino | SGSGSGTEFTLTISSLQSEDFAVYYCQQYNNWPFTFGPGTKV |
| | acid | DIKGGGGSGGGGSGGGGSGGGGSQVQLQQSGPGLVKPSQTLS |
| | sequence | LTCAISGDSVSRNSAVWNWIRQSPSRGLEWLGRTYYRSKWYN |
| | | DYAPSVKSRITINPDTSKNQFSLQLNSVTPEDTAVYYCARGL |
| | | RQNQFYYYMDVWGKGTTVTVSSGSESKYGPPCPPCPFWVLVV |
| | | VGGVLACYSLLVTVAFIIFWVKRGRKKLLYIFKQPFMRPVQT |
| | | TQEEDGCSCRFPEEEEGGCELRVKFSRSADAPAYQQGQNQLY |
| | | NELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 117 | STEAP2-2 | MLLLVTSLLLCELPHPAFLLIPDVLMTQTPLSLPVSLGDQAS |
| | BZ CAR- | ISCRSSQSVVHSNGNTYLEWYLQKPGQSPKLLIYKVSNRFSG |
| | T2A- | VPDRFSGSGSGTDFTLKISRVEAEDLGVYYCFQGSHVPYTFG |
| | Dominant- | GGTKLEIKGGGGSGGGGSGGGGSGGGGSEVQLVESGGDLVKP |
| | negative | GGSLKLSCAASGFSFSSYGMSWVRQTPDKRLEWVATISSGGS |
| | TGF-β | YTFYPDIMKGRFTISRDNAMNTLYLQMSSLKSEDSAMYYCAR |
| | receptor | RGYGTIYTFSFDSWGQGTTLTVSSGSESKYGPPCPPCPFWVL |
| | type 2 | VVVGGVLACYSLLVTVAFIIFWVKRGRKKLLYIFKQPFMRPV |
| | amino | QTTQEEDGCSCRFPEEEEGGCELRVKFSRSADAPAYQQGQNQ |
| | acid | LYNELNLGRREEYDVLDKRRGRDPEMGGKPRRKNPQEGLYNE |
| | sequence | LQKDKMAEAYSEIGMKGERRRGKGHDGLYQGLSTATKDTYDA |
| | | LHMQALPPRGSGEGRGSLLTCGDVEENPGPMGRGLLRGLWPL |
| | | HIVLWTRIASTIPPHVQKSVNNDMIVTDNNGAVKFPQLCKFC |
| | | DVRFSTCDNQKSCMSNCSITSICEKPQEVCVAVWRKNDENIT |
| | | LETVCHDPKLPYHDFILEDAASPKCIMKEKKKPGETFFMCSC |
| | | SSDECNDNIIFSEEYNTSNPDLLLVIFQVTGISLLPPLGVAI |
| | | SVIIIFYCYRVNRQ |
| 118 | STEAP2-2 | MLLLVTSLLLCELPHPAFLLIPDVVMTQSPLSLPVTLGQPAS |
| | Humanized1 | ISCRSSQSVVHSNANTYLEWYLQKPGQSPQLLIYKVSNRFSG |
| | BZ | VPDRFSGSGSGTDFTLKISRVEAEDVGVYYCFQGSHVPYTFG |
| | CAR | QGTKLEIKGGGGSGGGGSGGGGSGGGGSEVQLVESGGGLVKP |
| | amino | GGSLRLSCAASGFTFSSYGMSWVRQAPGKRLEWVATISSGGS |
| | acid | YTFYPDIMKGRFTISRDNSKNTLYLQMNSLRAEDTAVYYCAR |
| | sequence | RGYGTIYTFSFDAWGQGTTLTVSSGSESKYGPPCPPCPFWVL |
| | | VVVGGVLACYSLLVTVAFIIFWVKRGRKKLLYIFKQPFMRPV |
| | | QTTQEEDGCSCRFPEEEEGGCELRVKFSRSADAPAYQQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYDA |
| | | LHMQALPPRGSGEGRGSLLTCGDVEENPGPMGRGLLRGLWPL |
| | | HIVLWTRIASTIPPHVQKSVNNDMIVTDNNGAVKFPQLCKFC |
| | | DVRFSTCDNQKSCMSNCSITSICEKPQEVCVAVWRKNDENIT |
| | | LETVCHDPKLPYHDFILEDAASPKCIMKEKKKPGETFFMCSC |
| | | SSDECNDNIIFSEEYNTSNPDLLLVIFQVTGISLLPPLGVAI |
| | | SVIIIFYCYRVNRQ |
| 119 | STEAP2-2 | MLLLVTSLLLCELPHPAFLLIPDVVMTQSPLSLPVTLGQPAS |
| | Humanized2 | ISCRSSQSVVHSNANTYLEWYLQKPGQSPQLLIYKVSNRFSG |
| | BZ | VPDRFSGSGSGTDFTLKISRVEAEDVGVYYCFQGSHVPYTFG |
| | CAR | QGTKLEIKGGGGSGGGGSGGGGSGGGGSEVQLLESGGGLVQP |
| | amino | GGSLRLSCAASGFTFSSYGMSWVRQAPGKRLEWVSTISSGGS |
| | acid | YTFYPDIMKGRFTISRDNSKNTLYLQMNSLRAEDTAVYYCAR |
| | sequence | RGYGTIYTFSFDAWGQGTTLTVSSGSESKYGPPCPPCPFWVL |
| | | VVVGGVLACYSLLVTVAFIIFWVKRGRKKLLYIFKQPFMRPV |
| | | QTTQEEDGCSCRFPEEEEGGCELRVKFSRSADAPAYQQGQNQ |
| | | LYNELNLGRREEYDVLDKRRGRDPEMGGKPRRKNPQEGLYNE |
| | | LQKDKMAEAYSEIGMKGERRRGKGHDGLYQGLSTATKDTYDA |
| | | LHMQALPPRGSGEGRGSLLTCGDVEENPGPMGRGLLRGLWPL |
| | | HIVLWTRIASTIPPHVQKSVNNDMIVTDNNGAVKFPQLCKFC |
| | | DVRFSTCDNQKSCMSNCSITSICEKPQEVCVAVWRKNDENIT |
| | | LETVCHDPKLPYHDFILEDAASPKCIMKEKKKPGETFFMCSC |
| | | SSDECNDNIIFSEEYNTSNPDLLLVIFQVTGISLLPPLGVAI |
| | | SVIIIFYCYRVNRQ |
| 120 | STEAP2-3 | MLLLVTSLLLCELPHPAFLLIPEIVMTQSPATLSVSPGERAT |
| | BZ CAR- | LSCRASQSVSSNLAWYQQKPGQAPRLLIYGASTRATGIPARF |
| | T2A- | SGSGSGTEFTLTISSLQSEDFAVYYCQQYNNWPFTFGPGTKV |
| | Dominant- | DIKGGGGSGGGGSGGGGSGGGGSQVQLQQSGPGLVKPSQTLS |
| | negative | LTCAISGDSVSRNSAVWNWIRQSPSRGLEWLGRTYYRSKWYN |
| | TGF-β | DYAVSVKSRITINPDTSKNQFSLQLNSVTPEDTAVYYCARGL |
| | receptor | LQNQFYYYMDVWGKGTTVTVSSGSESKYGPPCPPCPFWVLVV |
| | type 2 | VGGVLACYSLLVTVAFIIFWVKRGRKKLLYIFKQPFMRPVQT |
| | amino | TQEEDGCSCRFPEEEEGGCELRVKFSRSADAPAYQQGQNQLY |
| | acid | NELNLGRREEYDVLDKRRGRDPEMGGKPRRKNPQEGLYNELQ |
| | sequence | KDKMAEAYSEIGMKGERRRGKGHDGLYQGLSTATKDTYDALH |
| | | MQALPPRGSGEGRGSLLTCGDVEENPGPMGRGLLRGLWPLHI |
| | | VLWTRIASTIPPHVQKSVNNDMIVTDNNGAVKFPQLCKFCDV |
| | | RFSTCDNQKSCMSNCSITSICEKPQEVCVAVWRKNDENITLE |
| | | TVCHDPKLPYHDFILEDAASPKCIMKEKKKPGETFFMCSCSS |
| | | DECNDNIIFSEEYNTSNPDLLLVIFQVTGISLLPPLGVAISV |
| | | IIIFYCYRVNRQ |
| 121 | STEAP2-4 | MLLLVTSLLLCELPHPAFLLIPDIQLTQSPSFLSASVGDRVT |
| | BZ CAR- | ITCRASQGISVYLAWYQQEPGKAPKLLIYAASTLQSGVPSRF |
| | T2A- | SGSGSGTEFTLTISSLQPEDFATYYCQQLNSYPRTFGQGTKV |
| | Dominant- | EIKGGGGSGGGGSGGGGSGGGGSEVQLVESGGGLVQPGGSLR |
| | negative | LSCAASGFTFSSFAMTWVRQAPGKGLEWVSVITYSGGRTYYA |
| | TGF-β | DSVKGRFTISRDNSKNTLYLQMNSLRAEDTAVYFCAKDRIAA |
| | receptor | VGPFDYWGQGTLVTVSSGSESKYGPPCPPCPFWVLVVVGGVL |
| | type 2 | ACYSLLVTVAFIIFWVKRGRKKLLYIFKQPFMRPVQTTQEED |
| | amino | GCSCRFPEEEEGGCELRVKFSRSADAPAYQQGQNQLYNELNL |
| | acid | GRREEYDVLDKRRGRDPEMGGKPRRKNPQEGLYNELQKDKMA |
| | sequence | EAYSEIGMKGERRRGKGHDGLYQGLSTATKDTYDALHMQALP |
| | | PRGSGEGRGSLLTCGDVEENPGPMGRGLLRGLWPLHIVLWTR |
| | | IASTIPPHVQKSVNNDMIVTDNNGAVKFPQLCKFCDVRFSTC |
| | | DNQKSCMSNCSITSICEKPQEVCVAVWRKNDENITLETVCHD |
| | | PKLPYHDFILEDAASPKCIMKEKKKPGETFFMCSCSSDECND |
| | | NIIFSEEYNTSNPDLLLVIFQVTGISLLPPLGVAISVIIIFY |
| | | CYRVNRQ |
| 122 | STEAP2-5 | MLLLVTSLLLCELPHPAFLLIPDIQMTQSPSTLSASVGDRVT |
| | BZ CAR- | ITCRASQSISRWLAWYQQKPGKAPKLLIYKASSLESGVPSRF |
| | T2A- | SGSGSGTEFTLTISSLQPDDFATYYCQQFNSFSPITFGQGTR |
| | Dominant- | LEIKGGGGSGGGGSGGGGSGGGGSQVQLVQSGAEVKKPGASV |
| | negative | KVSCKASGYTFTSYGISWVRQAPGQGLEWMGWISGYTGNTNY |
| | TGF-β | AQKLQGRVTMTADTSTSTAYMELRSLRSDDTAVYYCARGGSY |
| | receptor | FDYWGQGTLVTVSSGSESKYGPPCPPCPFWVLVVVGGVLACY |
| | type 2 | SLLVTVAFIIFWVKRGRKKLLYIFKQPFMRPVQTTQEEDGCS |
| | amino | CRFPEEEEGGCELRVKFSRSADAPAYQQGQNQLYNELNLGRR |
| | acid | EEYDVLDKRRGRDPEMGGKPRRKNPQEGLYNELQKDKMAEAY |
| | sequence | SEIGMKGERRRGKGHDGLYQGLSTATKDTYDALHMQALPPRG |
| | | SGEGRGSLLTCGDVEENPGPMGRGLLRGLWPLHIVLWTRIAS |
| | | TIPPHVQKSVNNDMIVTDNNGAVKFPQLCKFCDVRFSTCDNQ |
| | | KSCMSNCSITSICEKPQEVCVAVWRKNDENITLETVCHDPKL |
| | | PYHDFILEDAASPKCIMKEKKKPGETFFMCSCSSDECNDNI1 |
| | | FSEEYNTSNPDLLLVIFQVTGISLLPPLGVAISVIIIFYCYR |
| | | VNRQ |
| 123 | STEAP2-6 | MLLLVTSLLLCELPHPAFLLIPDVLMTQTPLSLPVSLGDQAS |
| | BZ CAR- | ISCRSSQSVVHSNGNTYLEWYLQKPGQSPKLLIYKVSNRFSG |
| | T2A- | VPDRFSGSGSGTDFTLKISRVEAEDLGVYYCFQGSHVPYTFG |
| | Dominant- | GGTKLEIKGGGGSGGGGSGGGGSGGGGSQVQLQQPGAELVKP |
| | negative | GASVKLSCKASGYTFTSYWMEWVKQRPGQGLEWIGMIHPNSG |
| | TGF-β | ITNYNERFKNKATLTVDKSSSTAYMQLSSLTSEDSAVYYCAR |
| | receptor | DHYYILAYWGOGTLVTVSAGSESKYGPPCPPCPFWVLVVVGG |
| | type 2 | VLACYSLLVTVAFIIFWVKRGRKKLLYIFKQPFMRPVQTTQE |
| | amino | EDGCSCRFPEEEEGGCELRVKFSRSADAPAYQQGQNQLYNEL |
| | acid | NLGRREEYDVLDKRRGRDPEMGGKPRRKNPQEGLYNELQKDK |
| | sequence | MAEAYSEIGMKGERRRGKGHDGLYQGLSTATKDTYDALHMQA |
| | | LPPRGSGEGRGSLLTCGDVEENPGPMGRGLLRGLWPLHIVLW |
| | | TRIASTIPPHVQKSVNNDMIVTDNNGAVKFPQLCKFCDVRFS |
| | | TCDNQKSCMSNCSITSICEKPQEVCVAVWRKNDENITLETVC |
| | | HDPKLPYHDFILEDAASPKCIMKEKKKPGETFFMCSCSSDEC |
| | | NDNIIFSEEYNTSNPDLLLVIFQVTGISLLPPLGVAISVIII |
| | | FYCYRVNRQ |
| 124 | STEAP2-7 | MLLLVTSLLLCELPHPAFLLIPDVVMTQTPLSLPVSLGNQAS |
| | BZ CAR- | ISCRSSQSLVHSNGNTYLHWYLQKPGQSPKLLIYKVSNRFSG |
| | T2A- | VPDRFSGSGSGTDFTLKISRVEAEDLGVYFCSQSTHVPLTFG |
| | Dominant- | AGTKLEIKGGGGSGGGGSGGGGSGGGGSQVQLQQPGADLVKP |
| | negative | GASVKMSCKASGHTFTNYWVTWVKQRPGQGLEWIGNFYPGSG |
| | TGF-β | IIKYNENFRSKATLTVDISSSTAYMQLSSLTSEDSAVYYCAR |
| | receptor | SKLGDSFYFDYWGQGTTLTVSSGSESKYGPPCPPCPFWVLVV |
| | type 2 | VGGVLACYSLLVTVAFIIFWVKRGRKKLLYIFKQPFMRPVQT |
| | amino | TQEEDGCSCRFPEEEEGGCELRVKFSRSADAPAYQQGQNQLY |
| | acid | NELNLGRREEYDVLDKRRGRDPEMGGKPRRKNPQEGLYNELQ |
| | sequence | KDKMAEAYSEIGMKGERRRGKGHDGLYQGLSTATKDTYDALH |
| | | MQALPPRGSGEGRGSLLTCGDVEENPGPMGRGLLRGLWPLHI |
| | | VLWTRIASTIPPHVQKSVNNDMIVTDNNGAVKFPQLCKFCDV |
| | | RFSTCDNQKSCMSNCSITSICEKPQEVCVAVWRKNDENITLE |
| | | TVCHDPKLPYHDFILEDAASPKCIMKEKKKPGETFFMCSCSS |
| | | DECNDNIIFSEEYNTSNPDLLLVIFQVTGISLLPPLGVAISV |
| | | IIIFYCYRVNRQ |
| 125 | STEAP2-8 | MLLLVTSLLLCELPHPAFLLIPEIVMTQSPATLSVSPGERAT |
| | BZ CAR- | LSCRASQSVASNLAWYQQKPGQAPRLLIYGASTRATGIPARF |
| | T2A- | SGSGSGTEFTLTISSLQSEDFAVYYCQQYNNWPFTFGPGTKV |
| | Dominant- | DIKGGGGSGGGGSGGGGSGGGGSQVQLQQSGPGLVKPSQTLS |
| | negative | LTCAISGDSVSRNSAVWNWIRQSPSRGLEWLGRTYYRSKWYN |
| | TGF-β | DYAPSVKSRITINPDTSKNQFSLQLNSVTPEDTAVYYCARGL |
| | receptor | RQNQFYYYMDVWGKGTTVTVSSGSESKYGPPCPPCPFWVLVV |
| | type 2 | VGGVLACYSLLVTVAFIIFWVKRGRKKLLYIFKQPFMRPVQT |
| | amino | TQEEDGCSCRFPEEEEGGCELRVKFSRSADAPAYQQGQNQLY |
| | acid | NELNLGRREEYDVLDKRRGRDPEMGGKPRRKNPQEGLYNELQ |
| | sequence | KDKMAEAYSEIGMKGERRRGKGHDGLYQGLSTATKDTYDALH |
| | | MQALPPRGSGEGRGSLLTCGDVEENPGPMGRGLLRGLWPLHI |
| | | VLWTRIASTIPPHVQKSVNNDMIVTDNNGAVKFPQLCKFCDV |
| | | RFSTCDNQKSCMSNCSITSICEKPQEVCVAVWRKNDENITLE |
| | | TVCHDPKLPYHDFILEDAASPKCIMKEKKKPGETFFMCSCSS |
| | | DECNDNIIFSEEYNTSNPDLLLVIFQVTGISLLPPLGVAISV |
| | | IIIFYCYRVNRQ |
| 126 | T2A | GSGEGRGSLLTCGDVEENPGP |
| | Peptide | |
| 127 | Signal | MLLLVTSLLLCELPHPAFLLIP |
| | Peptide | |
| 128 | IgG4P | ESKYGPPCPPCP |
| | Hinge | |
| | (S228P) | |
| 129 | CD28 | FWVLVVVGGVLACYSLLVTVAFIIFWV |
| | Transmembrane | |
| | Domain | |
| 130 | 4-1BB | KRGRKKLLYIFKQPFMRPVQTTQEEDGCSCRFPEEEEGGCEL |
| | Activation | |
| | Domain | |
| | (B domain) | |
| 131 | CD3zeta | RVKFSRSADAPAYQQGQNQLYNELNLGRREEYDVLDKRRGRD |
| | Domain | PEMGGKPRRKNPQEGLYNELQKDKMAEAYSEIGMKGERRRGK |
| | (z domain) | GHDGLYQGLSTATKDTYDALHMQALPPR |
| 132 | Bz | KRGRKKLLYIFKQPFMRPVQTTQEEDGCSCRFPEEEEGGCEL |
| | Domain | RVKFSRSADAPAYQQGQNQLYNELNLGRREEYDVLDKRRGRD |
| | | PEMGGKPRRKNPQEGLYNELQKDKMAEAYSEIGMKGERRRGK |
| | | GHDGLYQGLSTATKDTYDALHMQALPPR |
| 133 | CD28 Co- | RSKRSRLLHSDYMNMTPRRPGPTRKHYQPYAPPRDFAAYRS |
| | stimulatory | |
| | Domain | |

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1: Generation and Characterization of Anti-STEAP2 CARs

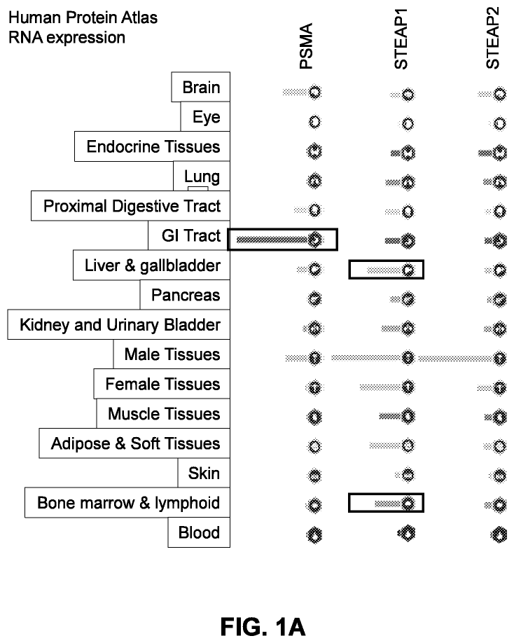

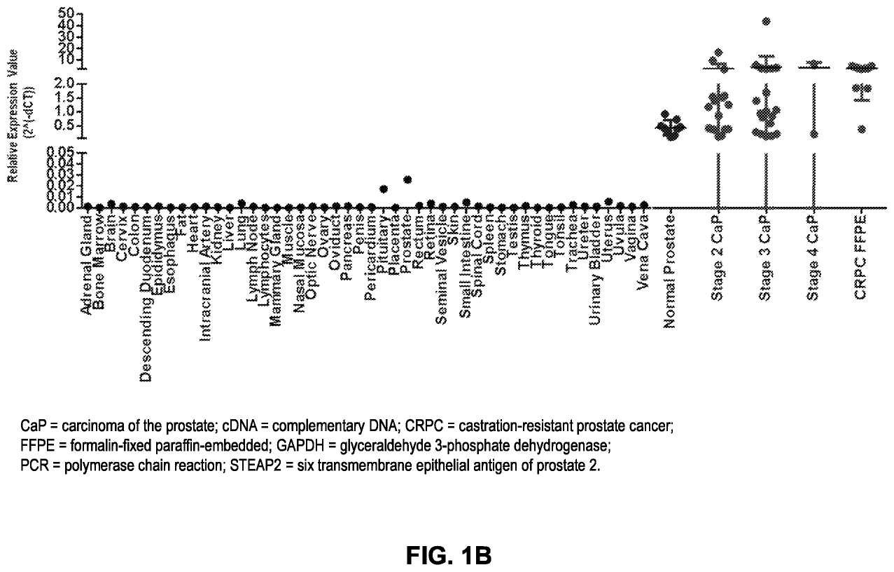

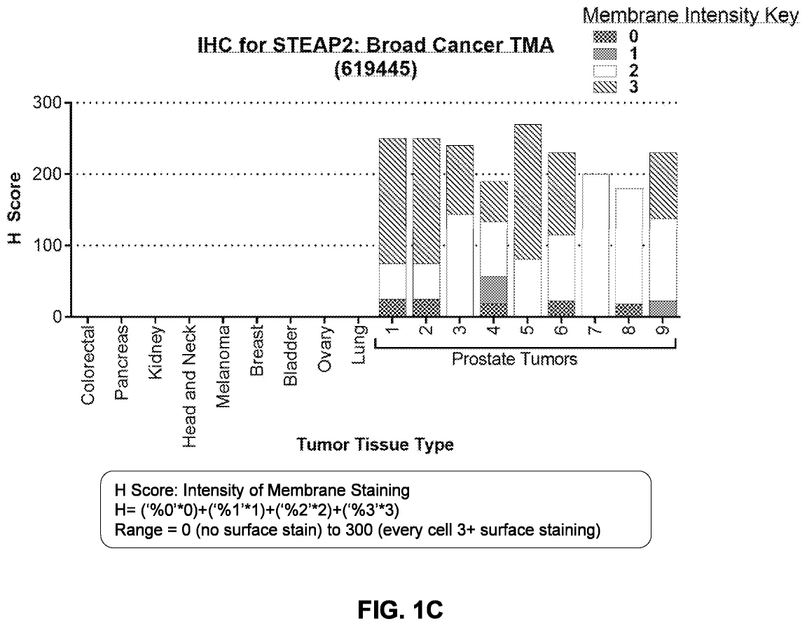

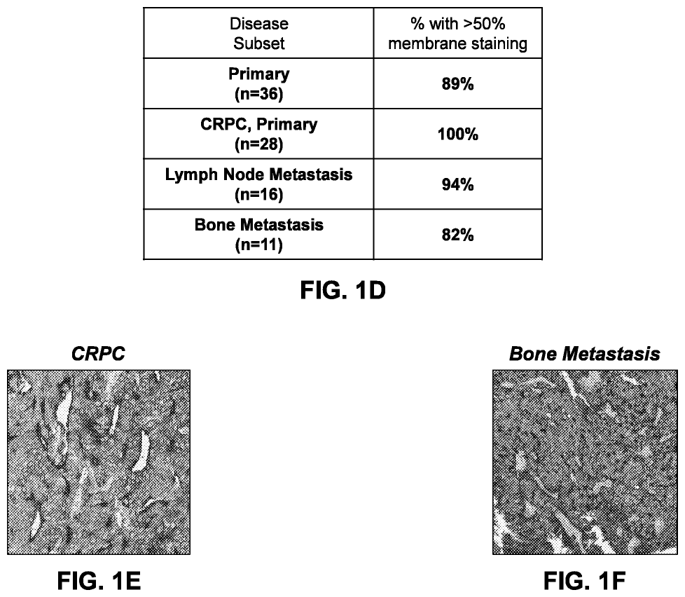

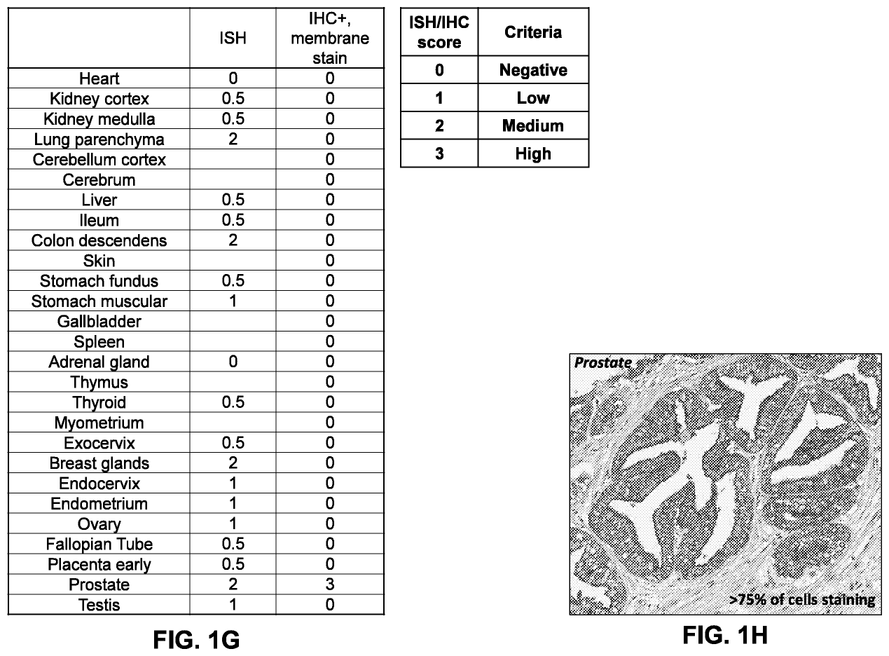

STEAP2 is a metalloreductase that reduces iron and copper to facilitate cellular uptake, metabolism, and proliferation, which is predominantly expressed in prostate cancer, with little to no expression in healthy tissue outside the prostate or in other cancer types (FIGS. 1A-1C). STEAP2 expression was analyzed by quantitative real-time PCR analysis of normal tissue and prostate cancer cDNA arrays. cDNA generated from CRPC FFPE patient samples was also included. GAPDH was utilized as a normalization control (FIG. 1B). STEAP2 has high, homogeneous cell surface expression across all disease stages, including metastases and CRPC (FIG. 1B), with a limited tissue sink and toxicity risk due to normal human tissue expression profile: prostate and minimal expression in kidney cortex (FIGS. 1A-1C). We performed cDNA array analyses, ISH, and IHC across a wide range of tumor types as well as prostate cancers throughout disease progression to confirm that the protein is overexpressed and present at the cell surface in prostate cancer compared to normal prostate (FIG. 1C).

IHC was performed on tissue microarrays containing primary prostate cancer, castrate-resistant prostate cancer (CRPC), and prostate lymph node metastases as well as decalcified full face sections of prostate cancer bone metastases for STEAP2 membrane expression (FIG. 1D). High expression of STEAP2 was found on CRPC (FIG. 1E) and in bone metastases (FIG. 1F). STEAP2 IHC and ISH performed in a normal human tissue microarray demonstrated absent or low STEAP2 in most normal tissues (FIG. 1G) and high STEAP 2 protein levels in normal prostate tissue (FIG. 1H).

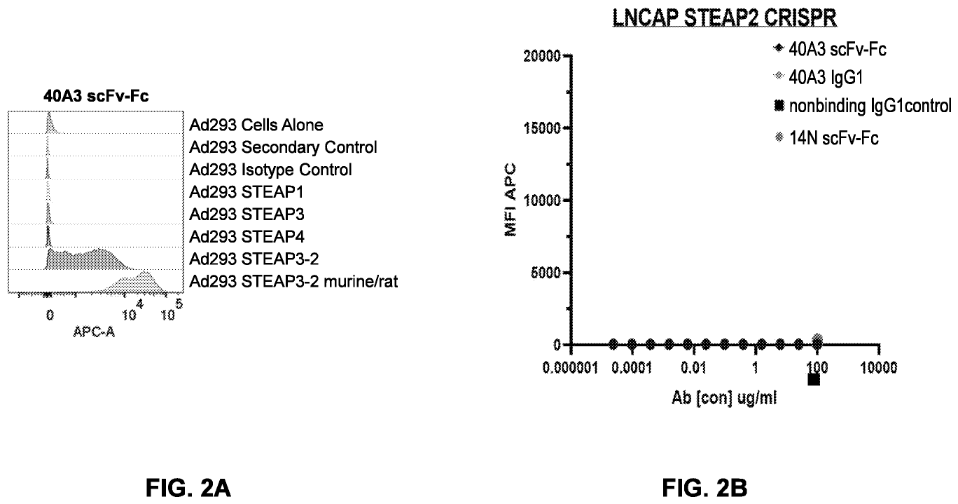

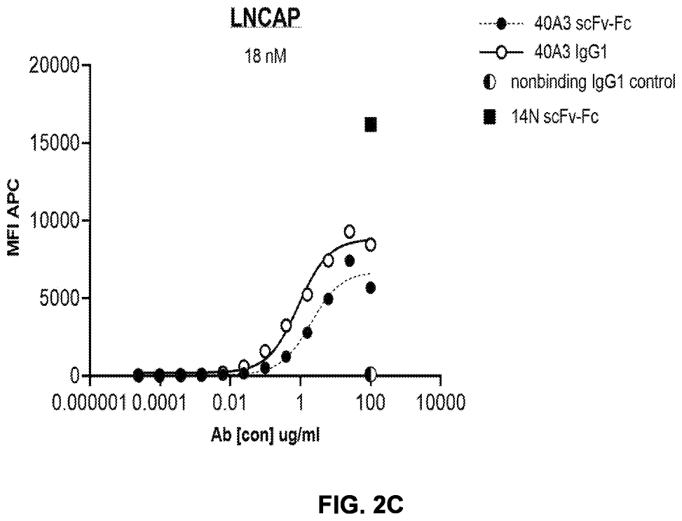

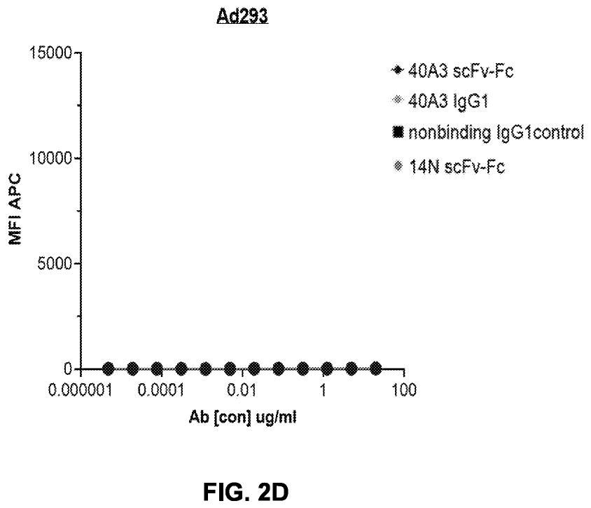

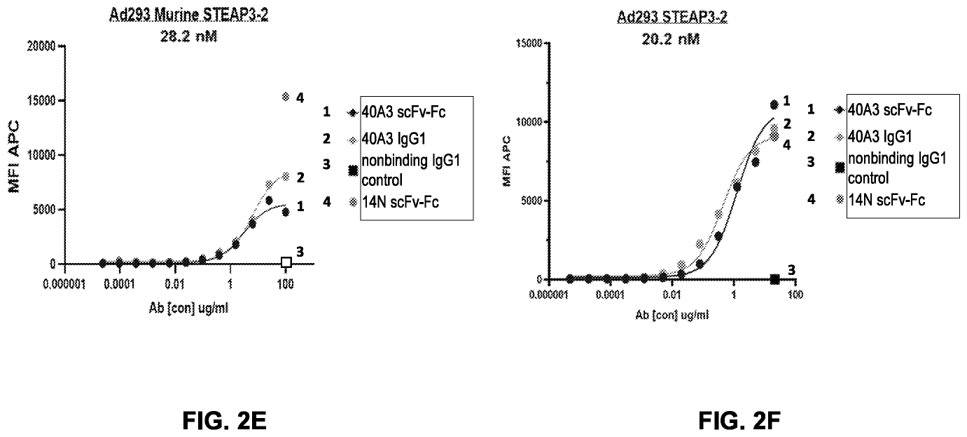

Multiple anti-STEAP2 antigen-binding constructs were generated and assayed for binding specificity. In vitro binding assays were performed to assess the specificity for STEAP2 (FIG. 2A) and on-cell binding affinities of candidate scFv-Fc's using antigen-positive and -negative cell lines (FIGS. 2B-2F). Clone 40A3 showed strong on-target binding to human, cyno, and murine/rat STEAP2 heterologously expressed by Ad293 cells (FIGS. 2A, 2E, and 2F), with little to no binding to control Ad293 cells (FIGS. 2A and 2D) or Ad293 cells expressing other STEAP family members (FIG. 2A). Clone 40A3 further bound LNCaP cells (androgen-sensitive human prostate adenocarcinoma cells), both as a scFv-Fc CAR construct and as an IgG1 antibody; binding which was lost in the STEAP2 knock-out LNCaP cells (FIG. 2B).

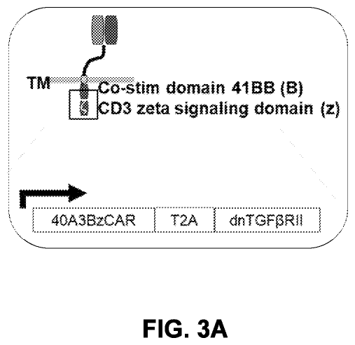

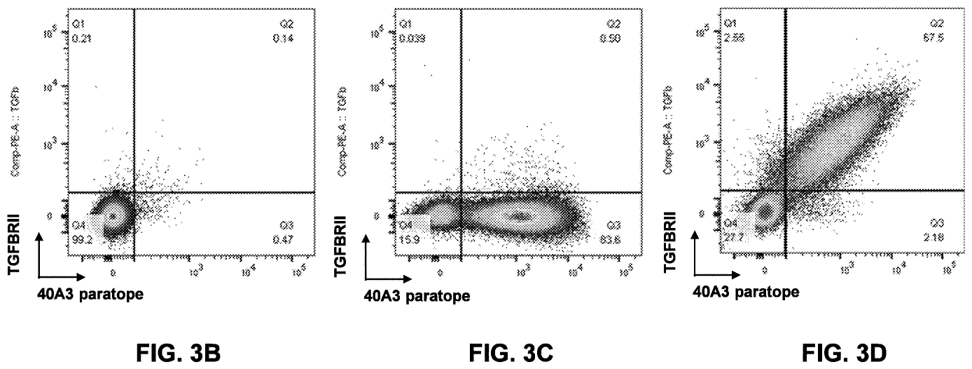

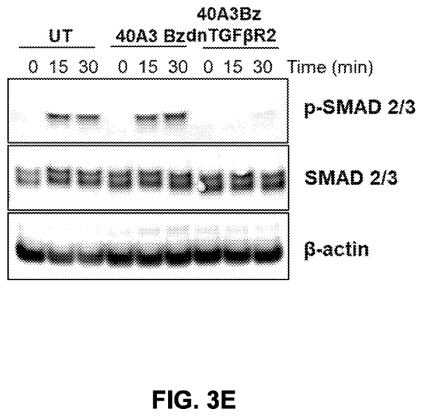

The antigen-binding domain of clone 40A3 was used to generate a CAR construct, referred to herein as 40A3Bz, by fusing the 40A3 antigen-binding domain to a 4-1BB costimulatory domain and a CD3 zeta signaling domain (FIG. 3A. top). To further enhance CAR-T efficacy in vivo, the CAR-encoding sequence was placed upstream of a sequence encoding a dominant negative TGFβRII (dnTGFβRII), linked by a viral T2A peptide-coding sequence (FIG. 3A, bottom). The T2A peptide allows for expression of two separate proteins from the same promoter, ensuring equimolar expression of both proteins. Lentiviral transduction was then used to express the CAR constructs with high efficiency (FIGS. 3B-3D). CAR-T cells were generated using CD3/CD28 bead activated donor T cells via lentiviral transduction. CAR-T cells were expanded in CAR-T culture conditions for 10 days prior to flow cytometry assessment of cell surface CAR expression compared to untransduced donor T cells. A paratope antibody recognizing 40A3Bz was utilized in conjunction with a TGFβRII antibody to evaluate the cell surface localization of 40A3Bz and 40A3Bz dnTGFβRII in untransduced (FIG. 3B), 40A3Bz (FIG. 3C), and 40A3Bz dnTGFβRII (FIG. 3D) transduced cell populations at day 10.

To provide functional validation of the dnTGFβRII armoring, CAR positive cells (40A3Bz STEAP2 CAR and 403Bz dnTGFβRII STEAP2 CAR-T cells) were purified to >97% purity through FACS at Day 4 post transduction. These cells were expanded to Day 15, starved in X VIVO™ 15 media for 17 hours prior to being stimulated with 1 ng/mL recombinant human TGFβ. Analysis of the acute signaling downstream of the native TGFβRII was compared between the armored and unarmored CAR-T cells in western blots for pSMAD 2/3, total SMAD 2/3, and 3-actin (FIG. 3E). A significant abrogation of TGFβ-mediated signaling in the dnTGFβRII (40A3Bz) CAR-T cells compared to 40A3Bz CAR or Untransduced cells alone was confirmed (FIG. 3E).

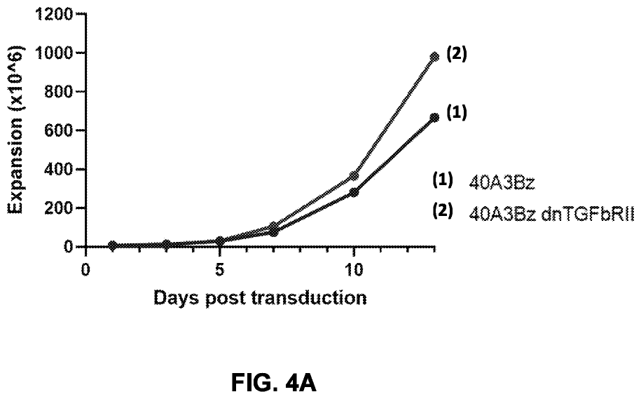

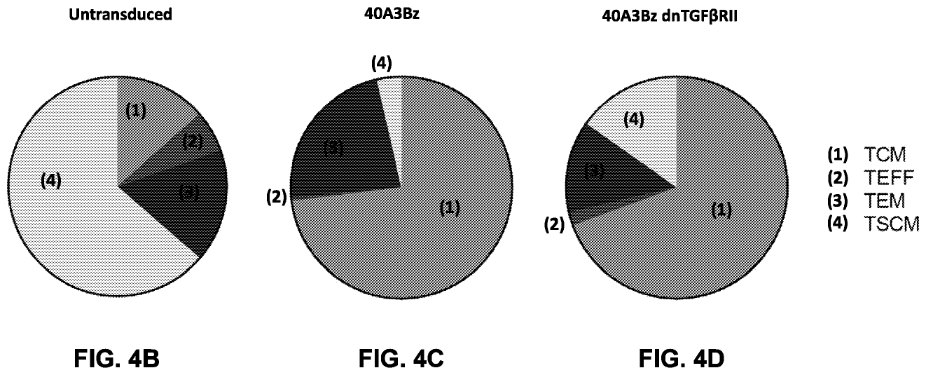

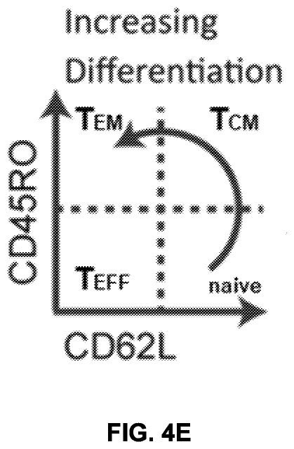

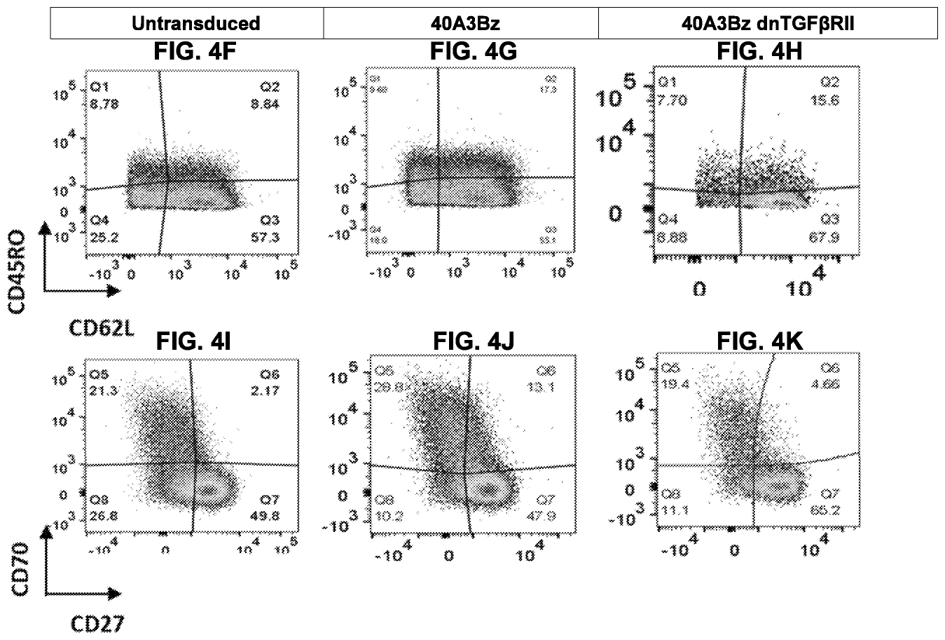

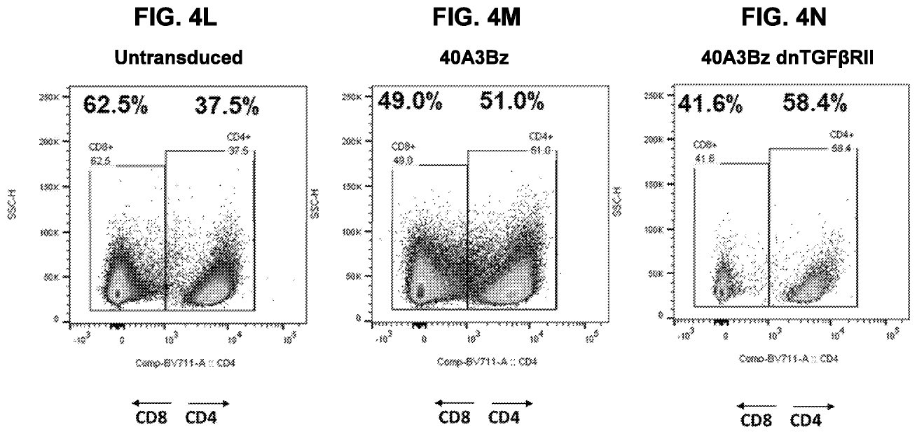

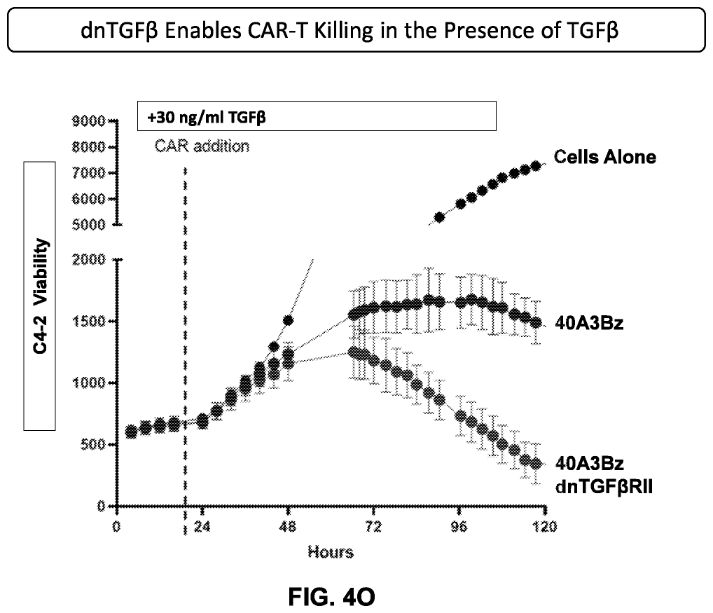

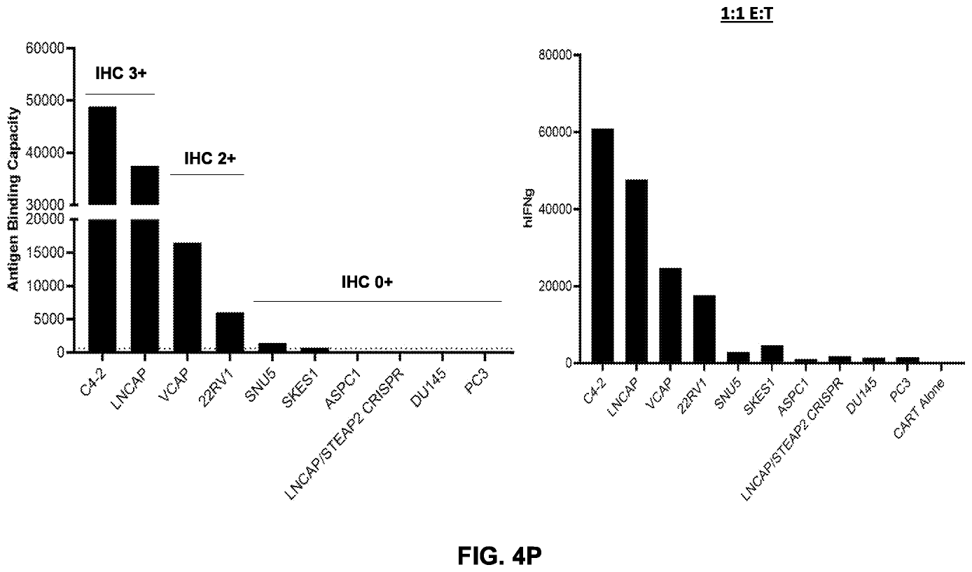

Phenotypic characterization was performed on (40A3Bz) STEAP2 CAR and dnTGFβRII (40A3Bz) STEAP2 CAR-T cells expanded from 2 healthy donors. T cells from all conditions were shown to have predominately a naive/central memory phenotype at the end of the expansion process at Day 10 as determined by staining with CD62L and CD45RO antibodies (FIGS. 4F-4K). Expanded CAR-T cells were also assessed for expression of differentiation and exhaustion markers (e.g., CD45RA, CD69, KLRG1, CD127, PD 1, and LAG-3; data not shown). Overall, the data showed that CAR and dnTGFβRII armoring can be expressed in T cells. Furthermore, it showed that the introduction of the dnTGFβRII armoring into the CAR constructs did not appear to substantially affect the cell phenotype at the end of culture.

Transduced 40A3Bz STEAP2 CAR and 40A3Bz dnTGFβRII STEAP2 CAR-T cells showed robust expansion (FIG. 4A). Phenotypic characterization was performed on 40A3Bz STEAP2 CAR and 40A3Bz dnTGFβRII STEAP2 CAR-T cells expanded from 2 healthy donors. T cells from all conditions were shown to have predominately a naïve/stem-like phenotype (FIGS. 4B-4K). Expanded CAR-T cells were also assessed for expression of differentiation and exhaustion markers (e.g., CD45RA, CD69, KLRG1, CD127, PD 1, and LAG-3; data not shown). Further, 40A3Bz STEAP2 CAR and 40A3Bz dnTGFβRII STEAP2 CAR-T cells maintained a mixed CD4:CD8 ratio (FIGS. 4L-4N). And 40A3Bz dnTGFβRII STEAP2 CAR-T cells were able to kill C4-2 target cells in the presence of 30 ng/ml TGFβ (FIG. 4O). 40A3Bz STEAP2 CAR and 40A3Bz dnTGFβRII STEAP2 CAR-T were pretreated with 30 ng/mL recombinant TGFβ for 6 days to suppress CAR-T function and then utilized in a co-culture assay with C4-2 cells stably expressing mKate red fluorescent protein at a 1:4 E:T ratio. Cytotoxicity was imaged using an Incucyte® live cell imaging system over time by the presence of RFP positive cells in co-culture over 120 hours. The results showed the improved potency of the dnTGFβRII armored CAR in vitro following exposure to immunosuppressive TGFβ.

A range of tumor cell lines was profiled by FACS with an anti-STEAP2 antibody-alexa fluor 647 conjugate for antibody binding capacity using the Bang's beads quantum simply cellular kit. STEAP2 cell surface IHC was performed on these cell lines and quantified (FIG. 4P, left panel). Further, these cell lines were included in 40A3Bz dnTGFβRII CAR-T co-culture assays at an E:T ratio of 1:1 and the media were sampled at 24 hours to analyze the levels of IFNγ release from the CAR-T cells. 40A3Bz dnTGFβRII CAR-T displayed specific cytotoxicity against C4-2, LNCaP, VCAP, 22RV1 cell lines (FIG. 4P, right panel).

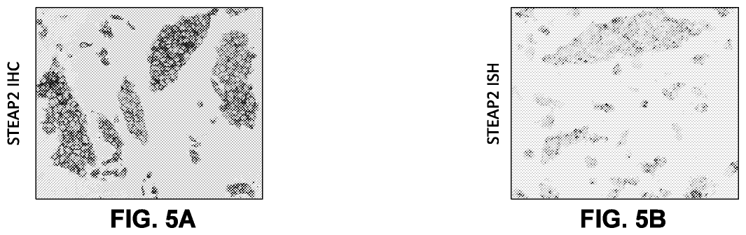

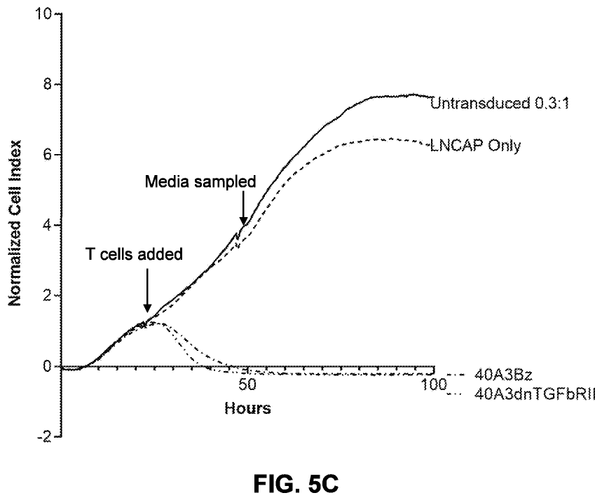

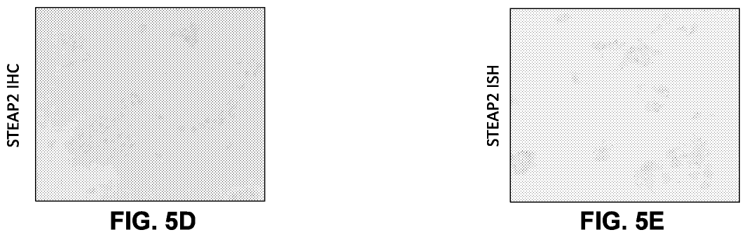

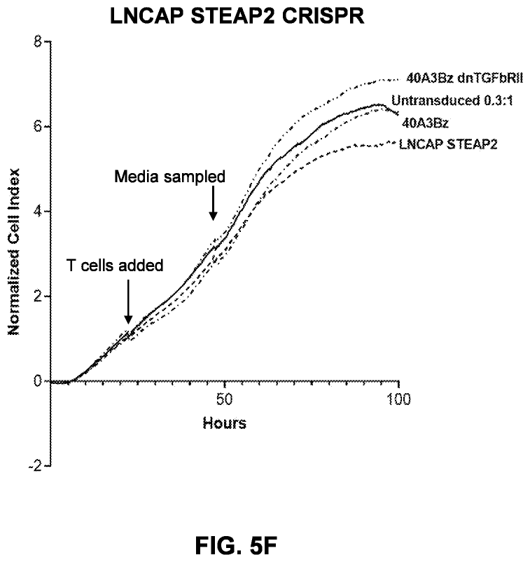

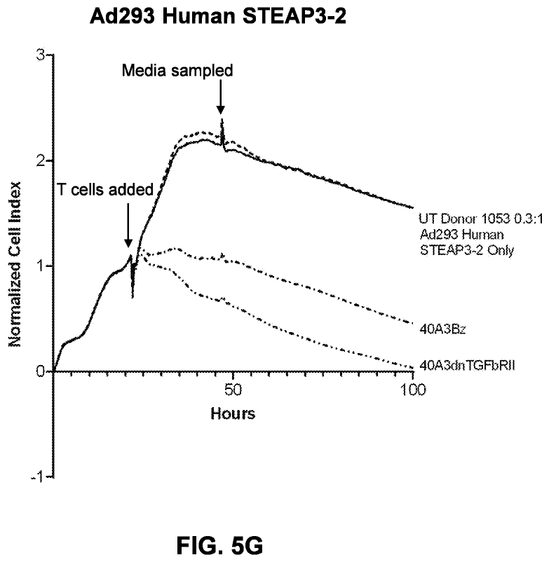

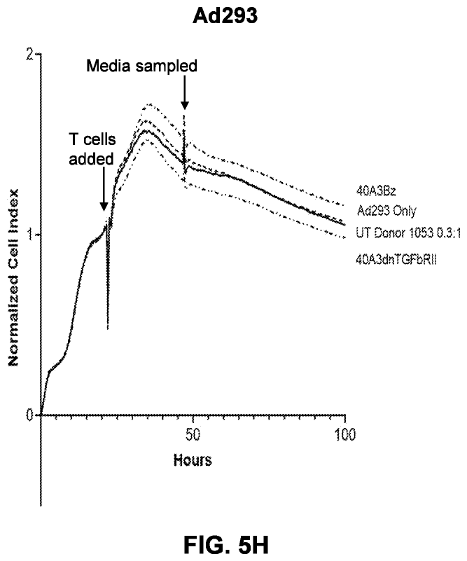

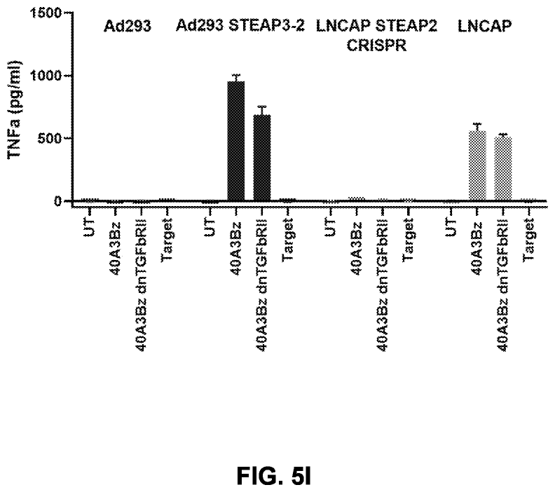

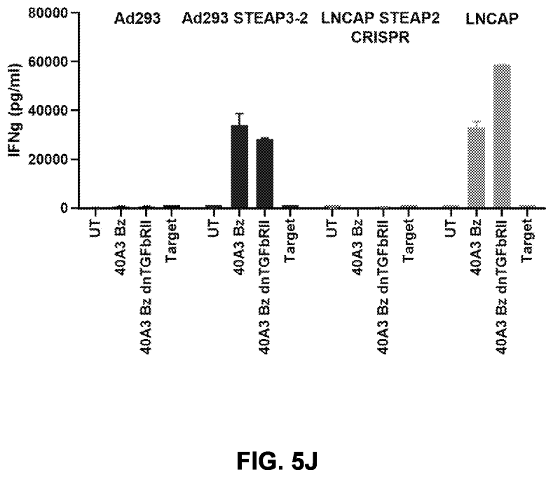

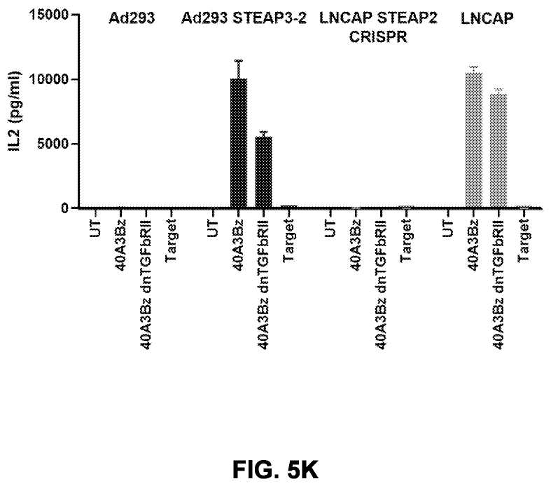

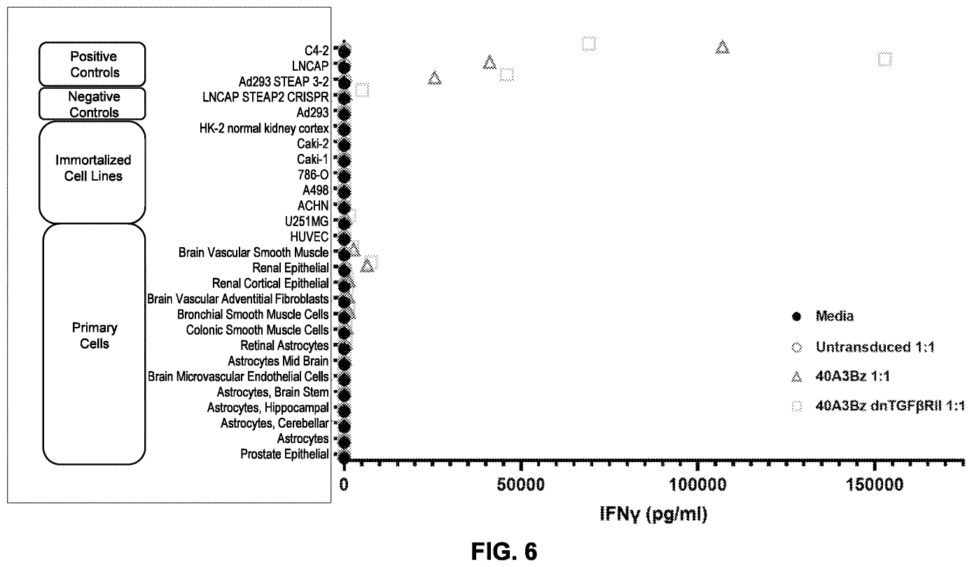

STEAP2 CAR's specific cytotoxicity over time was also determined. STEAP2 CAR-T cells were cytotoxic against LNCaP cells (FIGS. 5A-5C) and Ad293 cells that exogenously express human STEAP2 (FIG. 5G) but not against STEAP2 knock out LNCaP cells (FIG. 5D-5F) or control Ad293 cells (FIG. 5H). Dominant-negative TGFβRII 40A3Bz STEAP2 CAR-T cells expanded in human T-cell media (AIM V™ media supplemented with 5% Human AB Heat Inactivated Serum and 300 U/mL IL-2) for 10 days were shown to kill antigen-positive target cells in a similar fashion to unarmored STEAP2 CAR-T cells (FIGS. 5C, 5F, and 5G-5H). In addition, 40A3Bz and 40A3Bz dnTGFbRII STEAP2 CAR-T cells were shown to release pro-inflammatory cytokines after 24 hours of co culture (FIGS. 5I-5K). No cytotoxic activity and release of pro-inflammatory cytokines was observed following co-culture of armored STEAP2 CAR-T cells with antigen-negative targets. This indicates that T-cell activity is STEAP2 antigen-dependent with no evidence of tonic CAR signaling. STEAP2 CAR-T had minimal on-target, off-tumor activity (FIG. 6).

### Example 2: In Vivo Efficacy of Anti-STEAP2 CARs in a Mouse Model

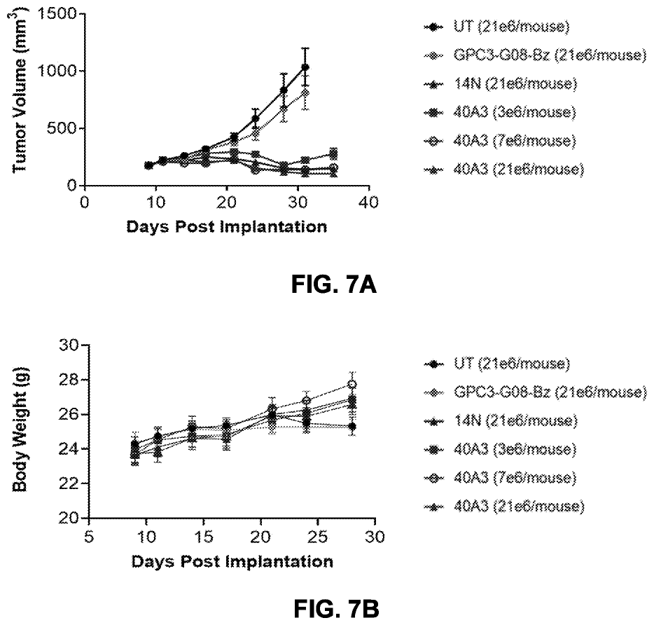

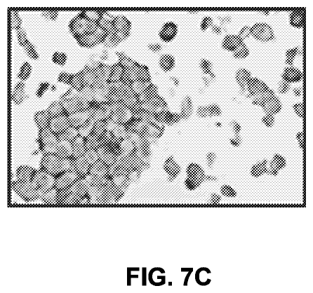

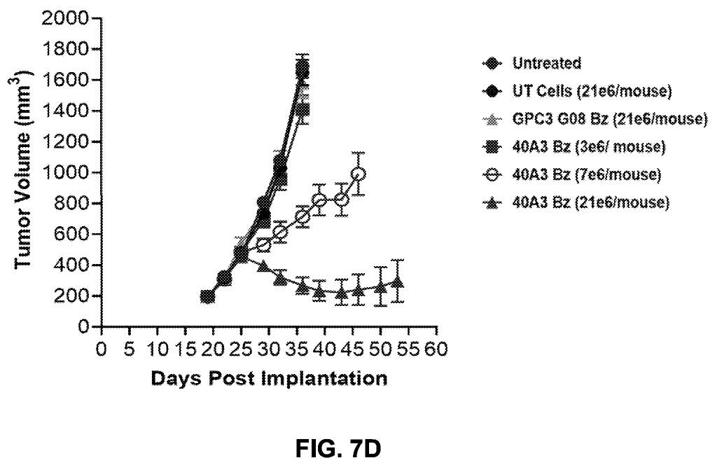

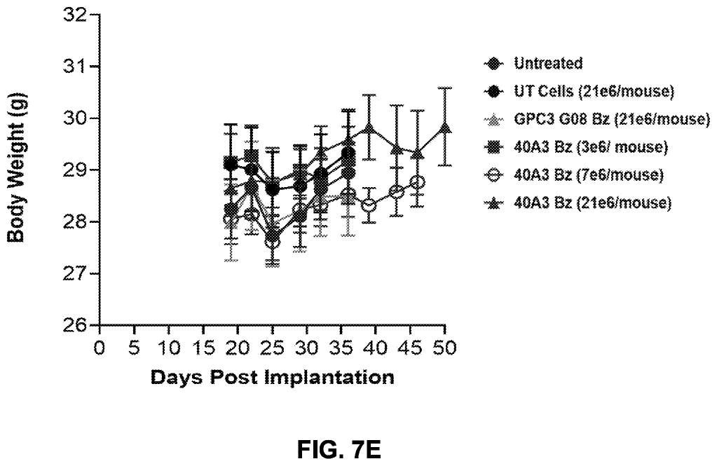

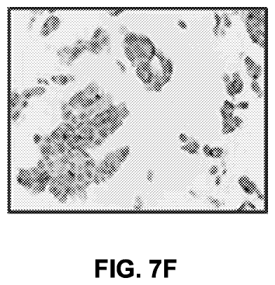

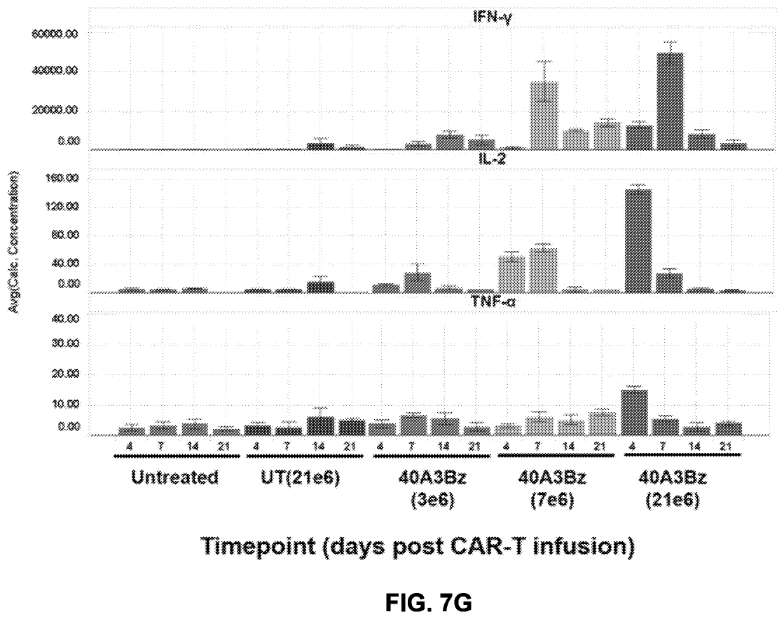

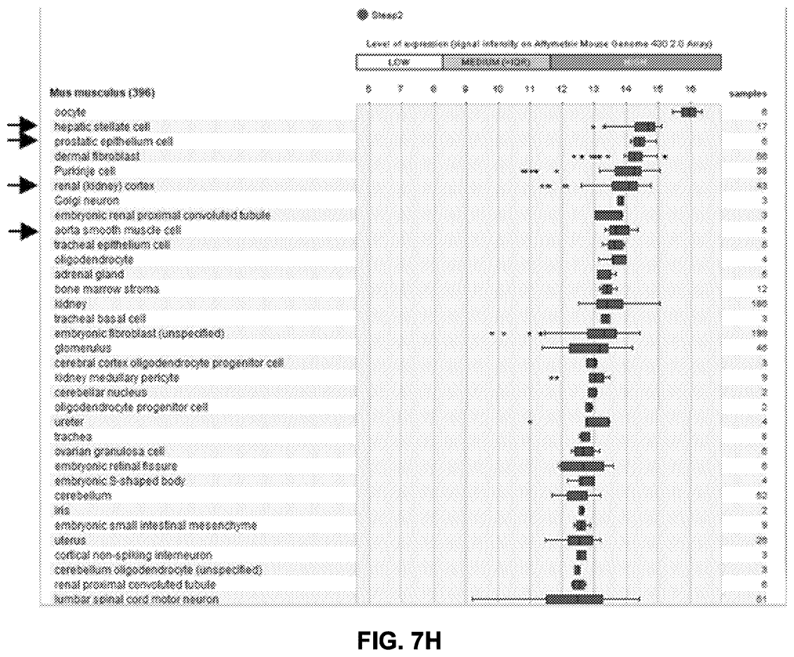

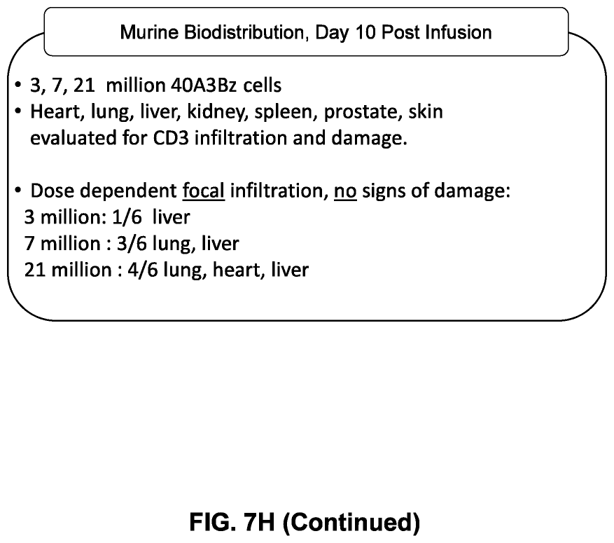

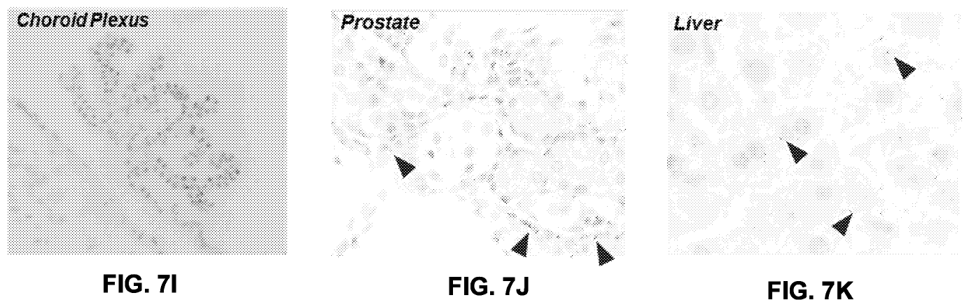

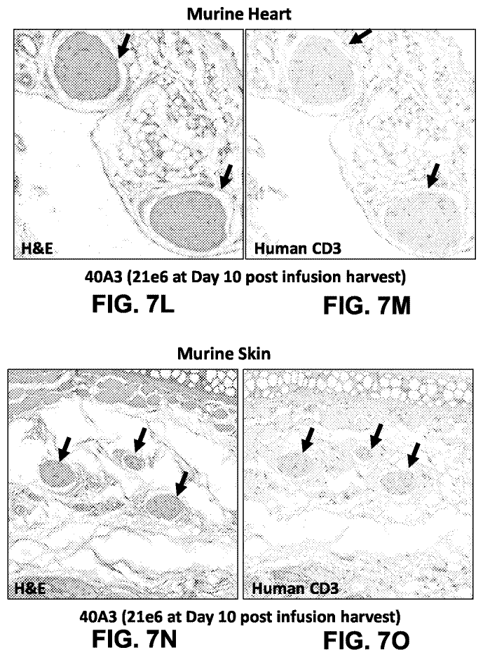

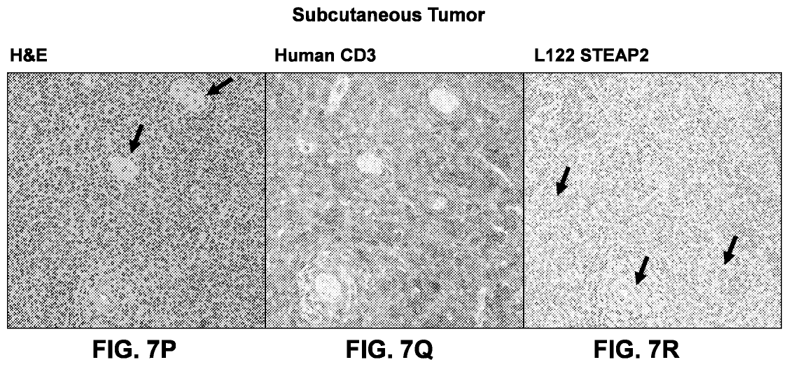

Mice were implanted with STEAP2 positive tumor cells and administered untransduced (UT) T cells or T cells transduced with a GPC3-G08-Bz negative control, a 14N positive control, or 3, 7, or 21 million 40A3Bz CAR-T cells (FIGS. 7A-7F). In vivo proof-of-concept studies were conducted using 2 prostate cancer cell lines, C4-2 and 22RV1 shown to express STEAP2 (FIGS. 7C and 7F). The data revealed control of C4-2 tumor in NSG mice following infusion of 40A3Bz STEAP2 CAR-T cells at 3 different doses (FIG. 7A). No adverse changes in mouse body weight were observed with all 3 CAR-T cell doses during the study (FIG. 7B). These data are relevant from a safety perspective given that the 40A3 scFv can cross-react with murine STEAP2. In the 22RV1 xenograft model that contains a lower STEAP2 receptor density and proportion of expression compared to C4-2, control of the tumor was only evident at the highest dose (21×10⁶) of 40A3Bz STEAP2 CAR-T cells (FIG. 7D). Partial responses were seen with the 7×10⁶ dose, and no effect on tumor cell growth was observed with the lowest 3×10⁶ T-cell dose. Similar to the C4-2 model, mice engrafted with the 22RV1 cell line and then administered with 3 concentration levels of STEAP2 CAR-T cells showed no adverse change in body weight during the study (FIG. 7E).

Mice from the 22RV1 xenograft study were bled on Days 4, 7, 14, and 21 following CAR-T cell infusion to evaluate the kinetics of cytokine production. Human IFNγ, IL-2, and TNFα levels were elevated in serum, peaking at Day 4 or Day 7 for most CAR-T cell doses before reducing at Days 14 and 21 (FIG. 7G). These data confirm tumor recognition by STEAP2 CAR-T cells following challenge with tumor cell lines expressing various levels of STEAP2 antigen in vivo. STEAP2 expression 10 days post infusion was evaluated via ISH, with dose dependent focal infiltration and no signs of damage (FIGS. 7I-7K).

STEAP2 CAR-T administration showed no evidence of CD3+ CAR-T infiltration into nerves at the base of the heart (FIGS. 7L-7M) and no evidence of CD3+ CAR-T infiltration into peripheral subcutaneous nerves (FIGS. 7N-7O). However, two small intact and non-infiltrated peripheral nerves entrapped within the dense CAR-T infiltrate were observed (FIG. 7P; arrows). Several small blood vessels were also observed within the tumor with minimal to mild STEAP2 staining (FIG. 7R; arrow). Despite very dense CD3+ CAR-T infiltrate (FIG. 7Q), the nerves are intact and did not appear to be affected.

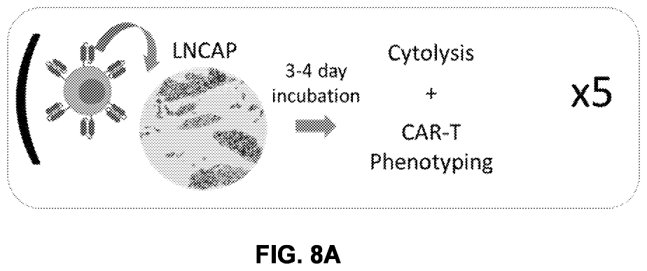

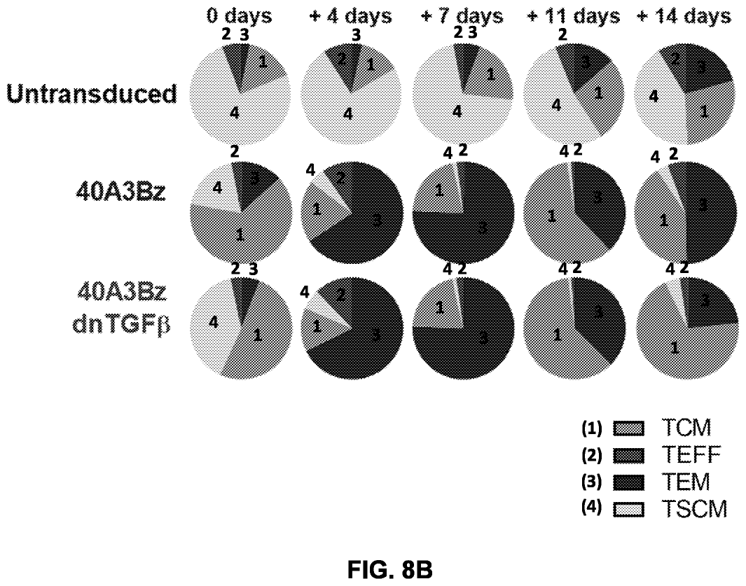

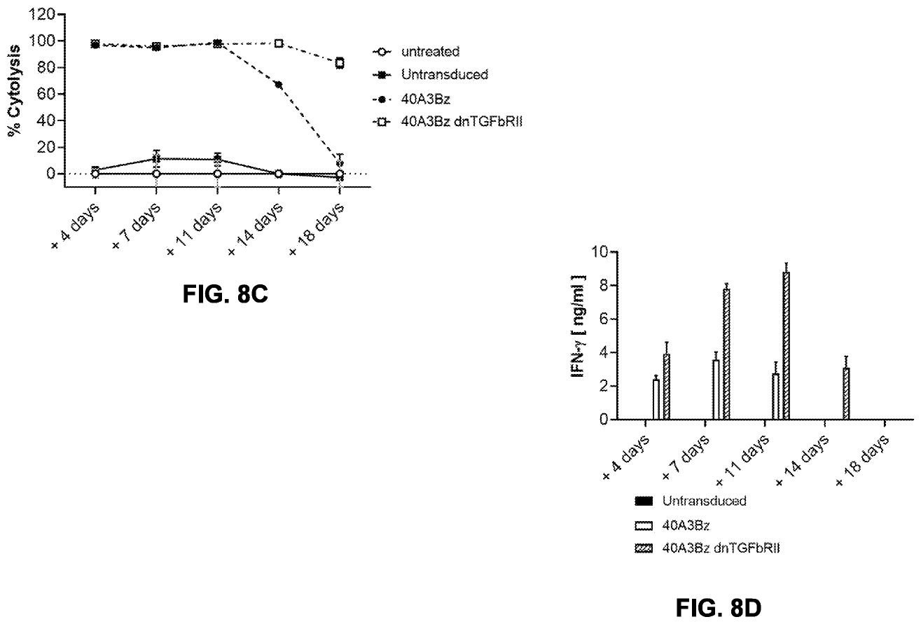

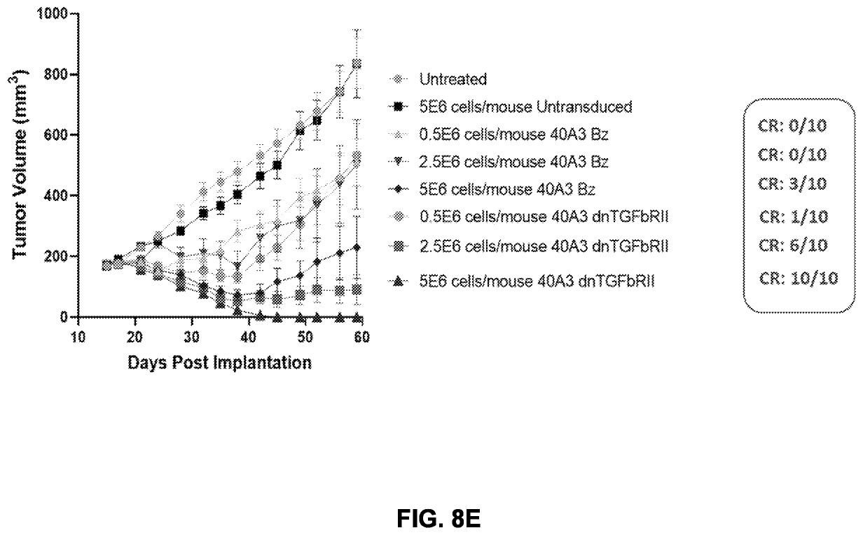

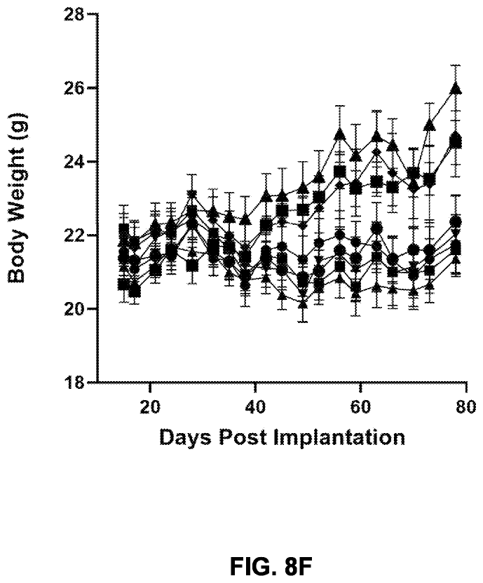

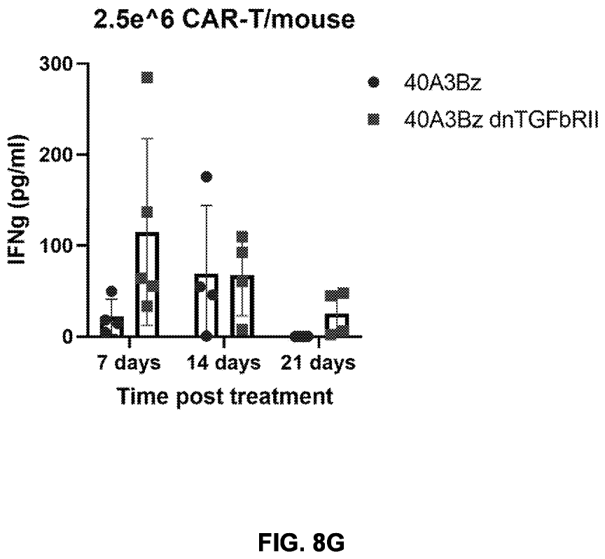

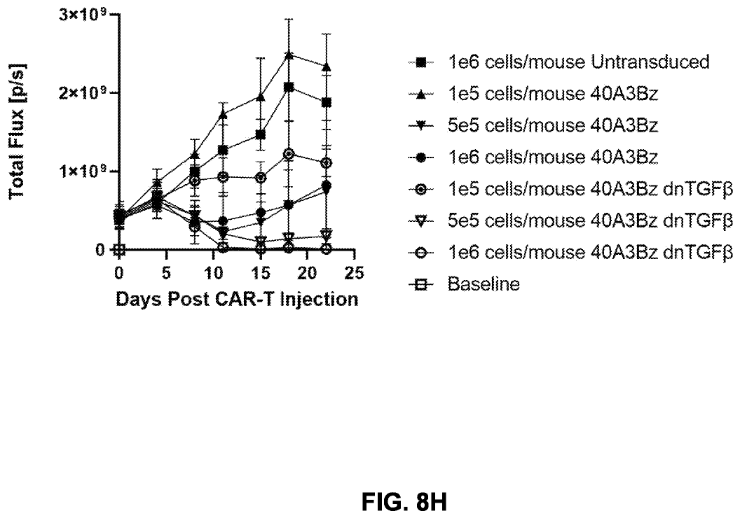

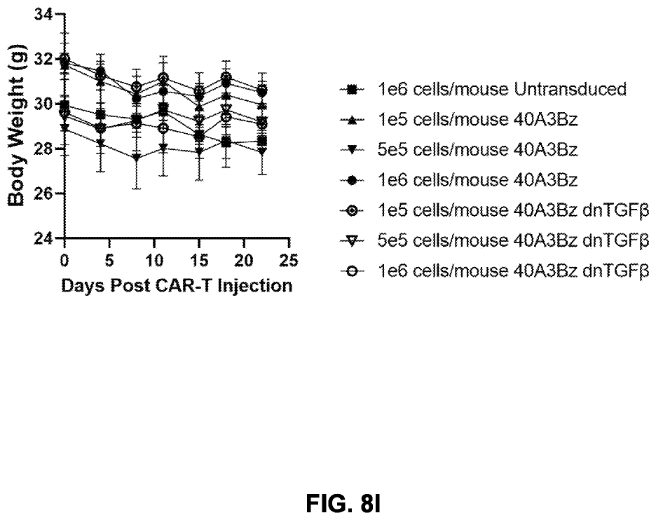

STEAP2 CAR-T showed increased persistence relative to untransduced T cells. Following 5 rounds of serial killing (FIG. 8A), 40A3Bz dnTGFβRII CAR-T cells showed consistent cytolysis and continuing IFN-γ production (FIGS. 8E-8G) in the presence of antigen positive cells, while maintaining a predominantly T_{CM} and T_{EM} differentiation status (FIG. 8B). 40A3Bz and 40A3Bz dnTGFβRII CAR-T cells dosed at 3 concentrations (0.5, 2.5, 5×e6 CAR positive cells) by tail vein injection in NSG mice implanted with TGFβ overexpressing C4-2 tumor cells showed reduced tumor volumes (FIG. 8E) and maintained body weights (FIG. 8F) out to 60 days post tumor implantation. Reduction in tumor volume (FIG. 8E) and cytokine release (FIGS. 8D and 8G) were further enhanced by dnTGFβRII armoring. Complete responders (CR) were defined as mice with a tumor volume of 0 mm3 for two successive measurements.

NSG mice were implanted in the intratibial space with C4-2 luciferase expressing cells and the luciferase signal was monitored. When the tumor flux reached 4.04e8 photons/sec, the animals were injected with 1×10⁶, 1×10⁵, 5×10⁵ or 1×10⁶ 40A3Bz CAR-T cells or 40A3Bz dnTGFβRII CAR-T cells per mouse. Tumor volume and body weights were monitored for 22 days post dose, and dose dependent tumor growth inhibition was evident with greater inhibition seen with the 40A3Bz dnTGFbRII CAR-T compared to 40A3Bz (FIG. 8H). No adverse change in body weight during the study was observed (FIG. 8I).

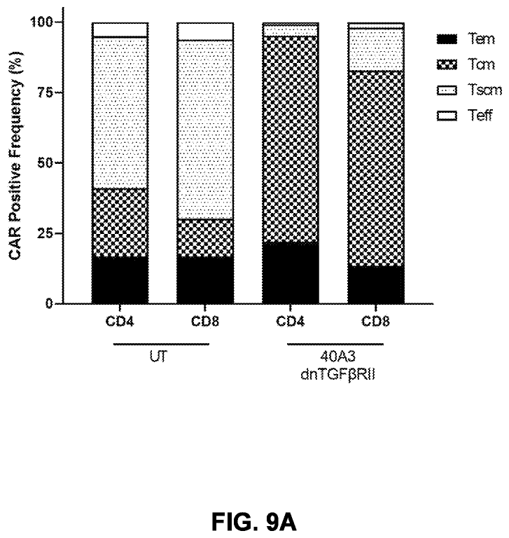

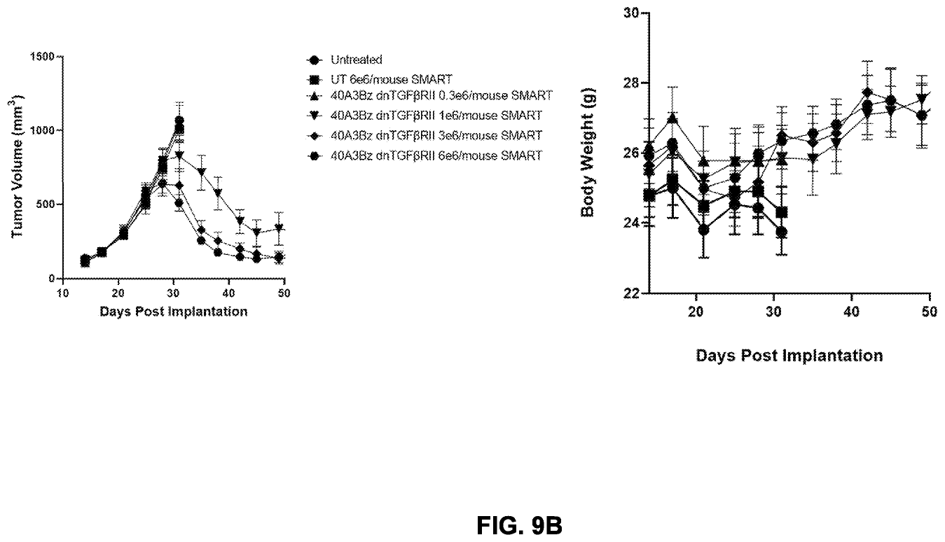

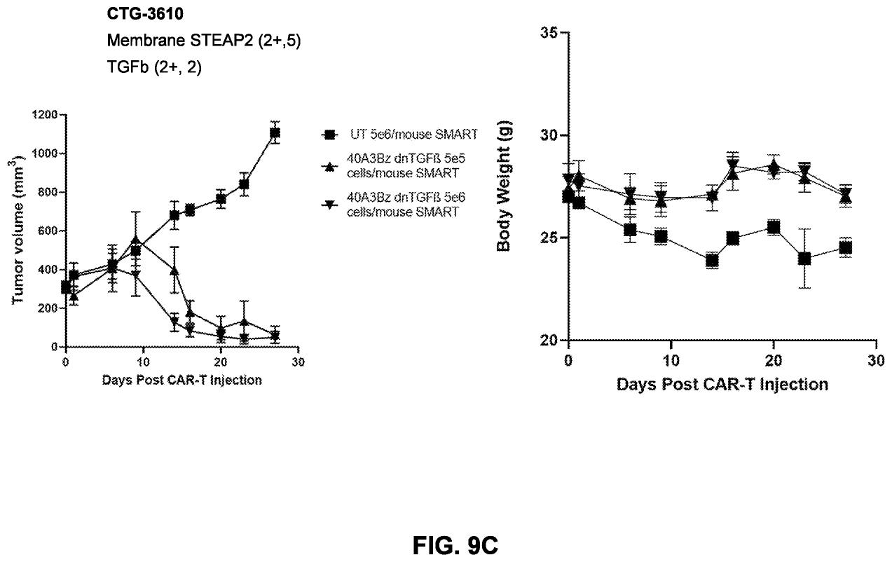

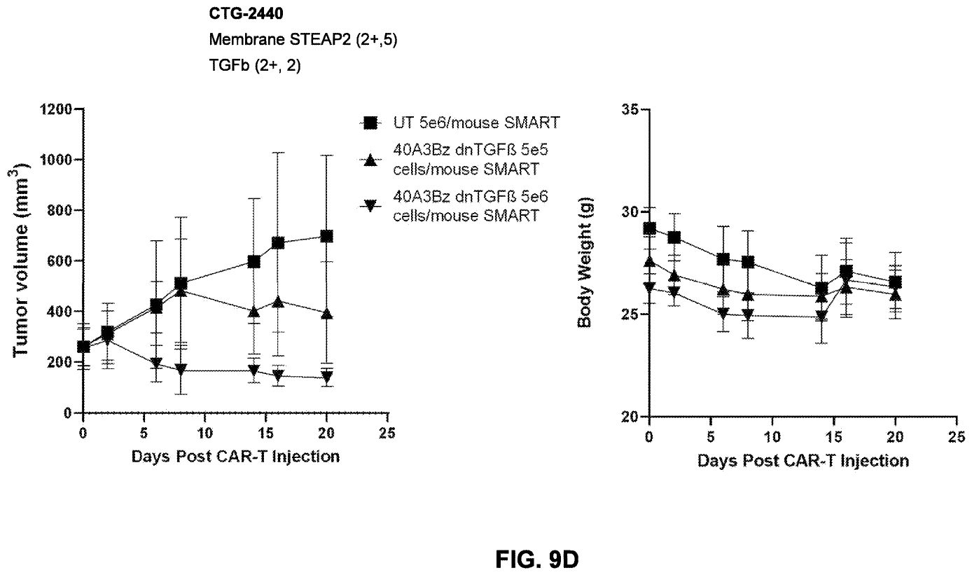

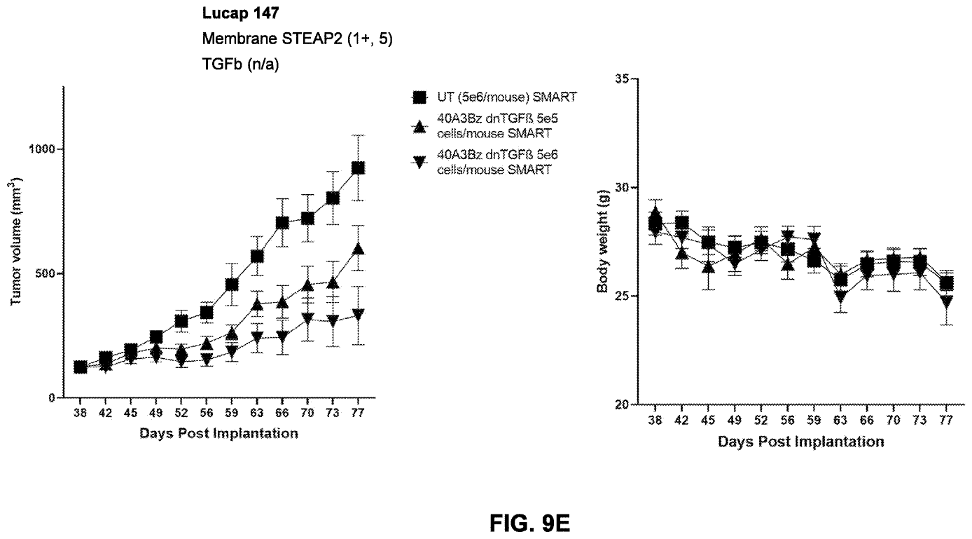

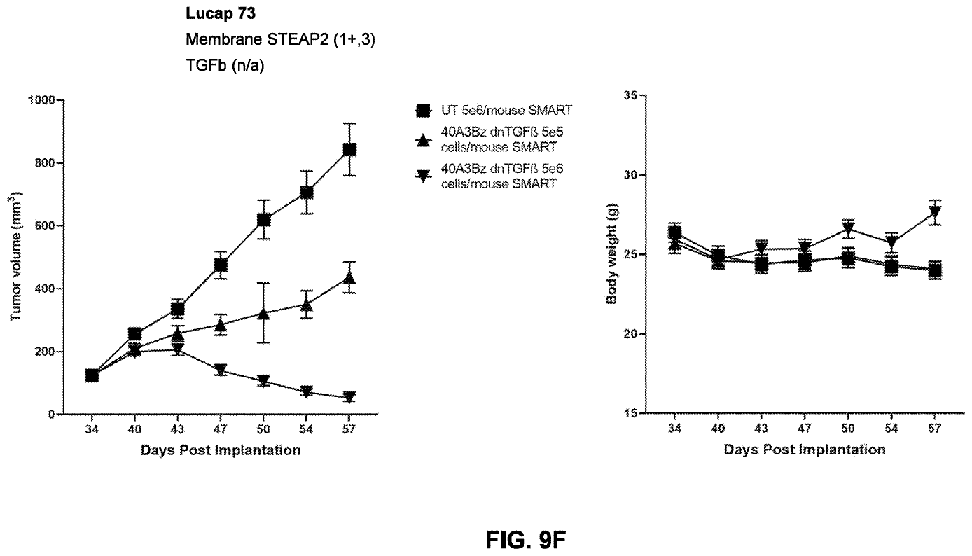

In some aspects, 40A3Bz dnTGFβRII CAR-T cells were manufactured according to the SMART process and CAR positivity, activation, and phenotypes of the cells were evaluated at expansion Day 4 and compared to untransduced T cells from the same donor (FIG. 9A). 40A3Bz dnTGFβRII SMART CAR-T cells were dosed at 4 concentrations (0.3, 1, 3, 6×10⁶ CAR positive cells) by tail vein injection in NSG MHC class 1 class 2 knockout mice implanted with 22Rv1 cells overexpressing TGFβ. Tumor volumes and body weights were measured out to 50 days post tumor implantation (FIG. 9B). Tumor volumes were effectively reduced in all 40A3Bz dnTGFβRII SMART CAR-T cell treated mice (FIG. 9B, top) with no adverse effects on body weight (FIG. 9B, bottom).

Further, NSG class 1 class 2 knockout mice were implanted with PDX fragments from frozen stocks of CTG-3610 prostate cancer cells and randomized when tumor volumes ranged from 125-250 mm³. The IHC intensity scores of CTB-3610 cells for membrane STEAP2 and TGFβ were 2+ and the proportion scores were 5 for STEAP2 and 2 for TGFβ (FIG. 9C). Mice were dosed with 0.5×10⁶ or 5×10⁶ 40A3Bz dnTGFβRII SMART CAR-T cells and compared to 5×10⁶ UT SMART controls. At both doses, the 40A3Bz dnTGFβRII SMART CAR-T cells efficiently suppressed tumor growth (FIG. 9C, top) with no adverse effect on body weight (FIG. 9C, bottom).

NSG class 1 class 2 knockout mice were implanted with PDX fragments from frozen stocks of CTG-2440 prostate cancer cells and randomized when tumor volumes ranged from 125-250 mm3. The IHC intensity scores for membrane STEAP2 and TGFβ were 2+ and the proportion scores were 5 for STEAP2 and 2 for TGFβ (FIG. 9D). Mice were dosed with 0.5×10⁶ or 5×10⁶ 40A3Bz dnTGFβRII SMART CAR-T cells and compared to 5×10⁶ UT SMART controls. The 40A3Bz dnTGFβRII SMART CAR-T cells suppressed tumor growth dose-dependently (FIG. 9D, top) with no adverse effect on body weight (FIG. 9D, bottom).

NSG class 1 class 2 knockout mice were implanted with PDX fragments from frozen stocks of Lucap 147 prostate cancer cells and randomized when tumor volumes ranged from 125-250 mm³. The IHC intensity score for membrane STEAP2 was 1+ and the proportion score for membrane STEAP2 was 5 (FIG. 9E). Mice were dosed with 0.5×10⁶ or 5×10⁶ 40A3Bz dnTGFβRII SMART CAR-T cells and compared to 5×10⁶ UT SMART controls. The 40A3Bz dnTGFβRII SMART CAR-T cells suppressed tumor growth dose-dependently (FIG. 9E, top) with no adverse effect on body weight (FIG. 9E, bottom).

NSG class 1 class 2 knockout mice were implanted with PDX fragments from frozen stocks of Lucap 73 prostate cancer cells and randomized when tumor volumes ranged from 125-250 mm³. The IHC intensity score for membrane STEAP2 was 1+ and the proportion score for membrane STEAP2 was 3 (FIG. 9F). Mice were dosed with 0.5×10⁶ or 5×10⁶ 40A3Bz dnTGFβRII SMART CAR-T cells and compared to 5×10⁶ UT SMART controls. The 40A3Bz dnTGFβRII SMART CAR-T cells suppressed tumor growth dose-dependently (FIG. 9F, top) with no adverse effect on body weight (FIG. 9F, bottom).

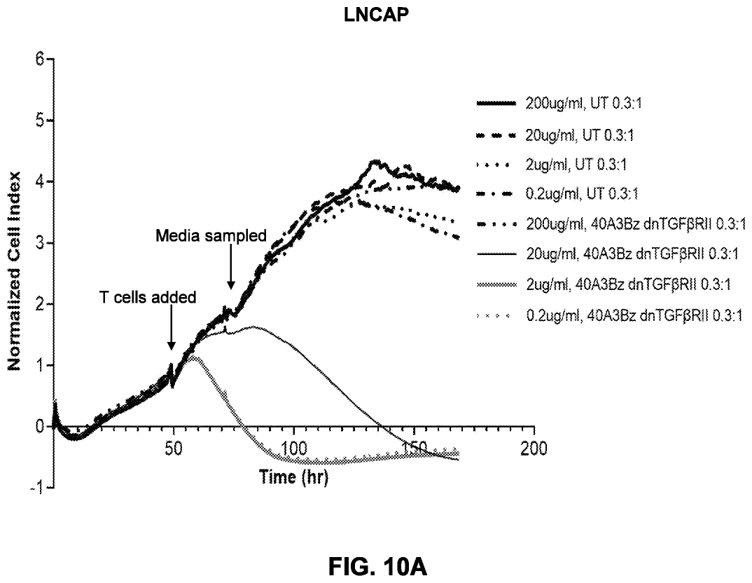

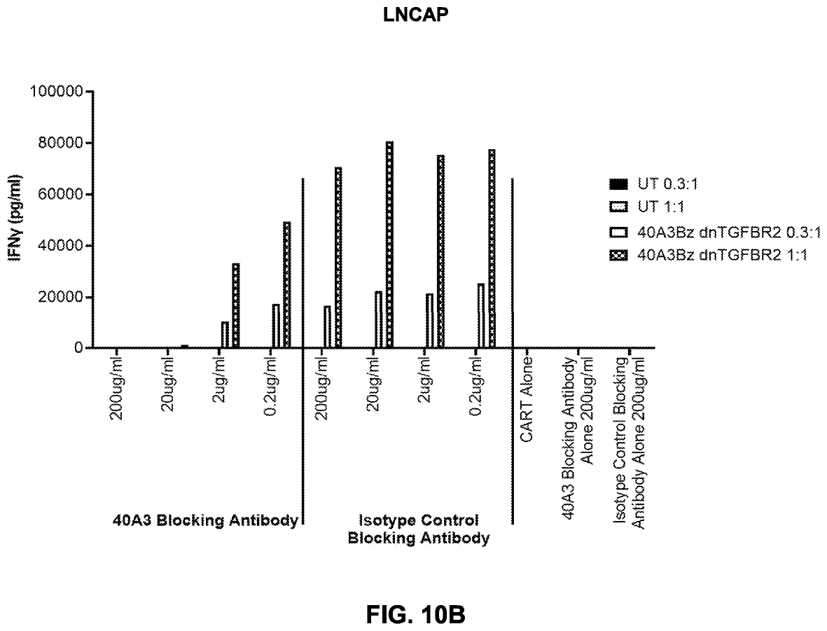

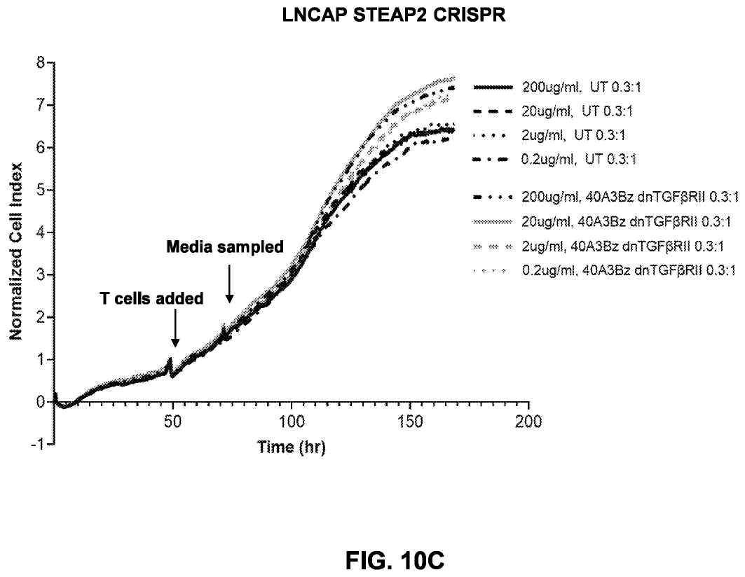

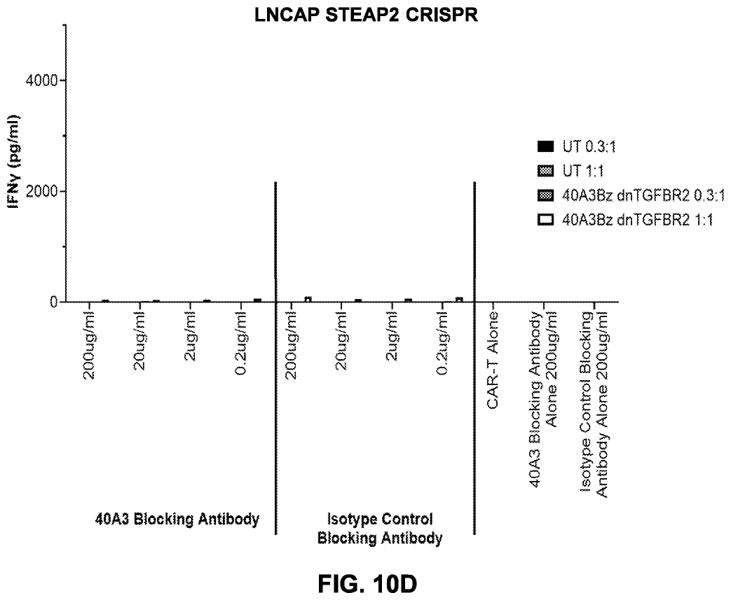

Co-cultures were setup with the LNCaP tumor cell line and 40A3Bz dnTGFβRII CAR-T cells at E:T ratio of 0.3:1 and blocking anti-STEAP2 antibodies were administered to the culture (0.2, 2, 20, or 200 μg/ml) (FIG. 10A). Co-cultures were also setup with the LNCaP tumor cell line and 40A3Bz dnTGFβRII CAR-T cells at E:T ratios of 0.3:1 and 1:1 in the presence of blocking anti-STEAP2 antibodies or isotype control blocking antibodies (0.2, 2, 20, or 200 μg/ml) and IFNγ levels were determined in the media (FIG. 10B). 40A3Bz dnTGFβRII CAR-T cells efficiently inhibited cell growth of LNCaP cells in the presence of low concentrations of blocking anti-STEAP2 antibodies. However, the highest concentration of anti-STEAP2 prevented the growth inhibition of LNCaP cells by 40A3Bz dnTGFβRII CAR-T cells (FIG. 10A). Similarly, IFNγ levels were induced dose-dependently by 40A3Bz dnTGFβRII CAR-T cells in the presence of low, but not high, concentrations of blocking STEAP2 antibodies, while isotype control blocking antibodies had no effect on IFNγ levels (FIG. 10B).

Co-cultures of LNCaP STEAP2 CRISPR cells with 40A3Bz dnTGFβRII CAR-T cells at E:T ratio of 0.3:1 in the presence of blocking anti-STEAP2 antibodies (0.2, 2, 20, or 200 μg/ml) showed similar cell growth of LNCaP STEAP2 CRISPR cells in the presence of 40A3Bz dnTGFβRII CAR-T cells as in the presence of untransduced T cells (FIG. 10C) and no secretion of IFNγ into the culture medium (FIG. 10D).

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All publications, patents, and patent applications disclosed herein are incorporated by reference to the same extent as if each individual publication, patent or patent application was specifically and individually indicated to be incorporated by reference.

What is claimed:

1. A polynucleotide comprising a nucleotide sequence encoding a chimeric antigen receptor (CAR), wherein the CAR comprises:
(i) an antigen-binding domain that binds an epitope on human six transmembrane epithelial antigen of prostate-2 (STEAP2);
(ii) a transmembrane domain; and
(iii) an intracellular domain.

2. The polynucleotide of claim 1, wherein the antigen-binding domain binds an epitope on an extracellular loop of human STEAP2.

3. The polynucleotide of claim 1 or 2, wherein the antigen-binding domain comprises an Fab, Fab′, F(ab′)2, Fd, Fv, single-chain fragment variable (scFv), single chain antibody, V_{H}H, vNAR, nanobody (single-domain antibody), or any combination thereof.

4. The polynucleotide of any one of claims 1 to 3, wherein the antigen-binding domain comprises an scFv.

5. The polynucleotide of any one of claims 1 to 4, wherein the antigen-binding domain comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3.

6. The polynucleotide of any one of claims 1 to 5, wherein the antigen-binding domain comprises a VH-CDR3 comprising an amino acid sequence selected from SEQ ID NOs: 6, 16, 26, 36, 46, and 96.

7. The polynucleotide of any one of claims 1 to 6, wherein the antigen-binding domain comprises a VH-CDR2 comprising an amino acid sequence selected from SEQ ID NOs: 5, 15, 25, 35, 45, and 95.

8. The polynucleotide of any one of claims 1 to 7, wherein the antigen-binding domain comprises a VH-CDR1 comprising an amino acid sequence selected from SEQ ID NOs: 4, 14, 24, 34, 44, and 94.

9. The polynucleotide of any one of claims 1 to 8, wherein the antigen-binding domain comprises a VL-CDR3 comprising an amino acid sequence selected from SEQ ID NOs: 3, 13, 23, 33, 43, and 93.

10. The polynucleotide of any one of claims 1 to 9, wherein the antigen-binding domain comprises a VL-CDR2 comprising an amino acid sequence selected from SEQ ID NOs: 2, 12, 22, 32, 42, and 92.

11. The polynucleotide of any one of claims 1 to 10, wherein the antigen-binding domain comprises a VL-CDR1 comprising an amino acid sequence selected from SEQ ID NOs: 1, 11, 21, 31, 41, and 91.

12. The polynucleotide of any one of claims 1 to 11, wherein the antigen-binding domain comprises:
(a) a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6;
(b) a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16;
(c) a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26;
(d) a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36;
(e) a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46; or
(f) a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 95, a VH-CDR3 comprising the amino acid sequence set forth in SEQ ID NO: 96.

13. The polynucleotide of any one of claims 1 to 12, wherein the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, and 97.

14. The polynucleotide of any one of claims 1 to 13, wherein the antigen-binding domain comprises a VH comprising an amino acid sequence selected from SEQ ID NOs: 7, 17, 27, 37, 47, and 97.

15. The polynucleotide of any one of claims 1 to 14, wherein the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, and 98.

16. The polynucleotide of any one of claims 1 to 15, wherein the antigen-binding domain comprises a VL comprising an amino acid sequence selected from SEQ ID NOs: 8, 18, 28, 38, 48, and 98.

17. The polynucleotide of any one of claims 1 to 12, wherein the antigen-binding domain comprises:
(a) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38;
(e) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48; or
(f) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

18. The polynucleotide of any one of claims 1 to 5, wherein the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57.

19. The polynucleotide of any one of claims 1 to 5 and 18, wherein the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58.

20. The polynucleotide of any one of claims 1 to 5, wherein the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67.

21. The polynucleotide of any one of claims 1 to 5 and 20, wherein the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68.

22. The polynucleotide of any one of claims 1 to 5, wherein the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77.

23. The polynucleotide of any one of claims 1 to 5 and 22, wherein the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78.

24. The polynucleotide of any one of claims 1 to 5, wherein the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87.

25. The polynucleotide of any one of claims 1 to 5 and 24, wherein the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88.

26. The polynucleotide of any one of claims 1 to 5, wherein the antigen-binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97.

27. The polynucleotide of any one of claims 1 to 5 and 26, wherein the antigen-binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

28. The polynucleotide of any one of claims 1 to 27, wherein the antigen-binding domain comprises:
(a) a VH comprising the amino acid sequence set forth in SEQ ID NO: 7, and a VL comprising the amino acid sequence set forth in SEQ ID NO: 8;
(b) a VH comprising the amino acid sequence set forth in SEQ ID NO: 17, and a VL comprising the amino acid sequence set forth in SEQ ID NO: 18;
(c) a VH comprising the amino acid sequence set forth in SEQ ID NO: 27, and a VL comprising the amino acid sequence set forth in SEQ ID NO: 28;
(d) a VH comprising the amino acid sequence set forth in SEQ ID NO: 37, and a VL comprising the amino acid sequence set forth in SEQ ID NO: 38;
(e) a VH comprising the amino acid sequence set forth in SEQ ID NO: 47, and a VL comprising the amino acid sequence set forth in SEQ ID NO: 48;
(f) a VH comprising the amino acid sequence set forth in SEQ ID NO: 57, and a VL comprising the amino acid sequence set forth in SEQ ID NO: 58;
(g) a VH comprising the amino acid sequence set forth in SEQ ID NO: 67, and a VL comprising the amino acid sequence set forth in SEQ ID NO: 68;
(h) a VH comprising the amino acid sequence set forth in SEQ ID NO: 77, and a VL comprising the amino acid sequence set forth in SEQ ID NO: 78;
(i) a VH comprising the amino acid sequence set forth in SEQ ID NO: 87, and a VL comprising the amino acid sequence set forth in SEQ ID NO: 88; or
(j) a VH comprising the amino acid sequence set forth in SEQ ID NO: 97, and a VL comprising the amino acid sequence set forth in SEQ ID NO: 98.

29. The polynucleotide of any one of claims 1 to 17 and 28, wherein the antigen-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9.

30. The polynucleotide of any one of claims 1 to 17 and 28, wherein the antigen-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 99.

31. The polynucleotide of any one of claims 1 to 30, wherein the intracellular domain comprises a costimulatory domain or a portion thereof.

32. The polynucleotide of any one of claims 1 to 31, wherein the intracellular domain comprises a costimulatory domain selected from the group consisting of the intracellular domain of CD3z, a CD28 co-stimulatory domain, a CD27 co-stimulatory domain, a 4-1BB co-stimulatory domain, an ICOS co-stimulatory domain, an OX-40 co-stimulatory domain, a GITR co-stimulatory domain, a CD2 co-stimulatory domain, an IL-2Rβ co-stimulatory domain, an MyD88/CD40a CD28 co-stimulatory domain, and any combination thereof.

33. The polynucleotide of any one of claims 1 to 32, wherein the intracellular domain comprises a 4-1BB co-stimulatory domain.

34. The polynucleotide of any one of claims 1 to 32, wherein the intracellular domain comprises the intracellular domain of CD3z and a CD28 co-stimulatory domain.

35. The polynucleotide of any one of claims 1 to 32, wherein the intracellular domain comprises the intracellular domain of CD3z comprising SEQ ID NO: 131 and a 4-1BB co-stimulatory domain comprising SEQ ID NO: 130.

36. The polynucleotide of any one of claims 1 to 35, wherein the intracellular domain comprises the intracellular domain of CD3z, a CD28 co-stimulatory domain, and a 4-1BB co-stimulatory domain.

37. The polynucleotide of any one of claims 1 to 36, wherein the transmembrane domain comprises a transmembrane domain selected from the transmembrane domain of CD4, CD8a, or CD28.

38. The polynucleotide of any one of claims 1 to 37, wherein the transmembrane domain comprises the transmembrane domain of CD28 and comprises SEQ ID NO: 129.

39. The polynucleotide of any one of claims 1 to 38, wherein the CAR further comprises a hinge/spacer domain.

40. The polynucleotide of claim 39, wherein the hinge/spacer domain comprises an immunoglobulin hinge/spacer.

41. The polynucleotide of claim 39 or 40, wherein the hinge/spacer domain comprises an IgG hinge domain.

42. The polynucleotide of any one of claims 39 to 41, wherein the hinge/spacer domain comprise an IgG1 hinge domain, an IgG2 hinge domain, an IgG3 hinge domain, or an IgG4 hinge domain.

43. The polynucleotide of any one of claims 39 to 42, wherein the hinge/spacer domain comprises an IgG4 hinge domain and comprises SEQ ID NO: 128.

44. The polynucleotide of any one of claims 1 to 43 wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10.

45. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 108.

46. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 109.

47. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 110.

48. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 111.

49. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 112.

50. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 113.

51. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 114.

52. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 115.

53. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 116.

54. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 118.

55. The polynucleotide of any one of claims 1 to 43, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 119.

56. The polynucleotide of any one of claims 1 to 55, which further encodes an armoring molecule, wherein the armoring molecule counters immunosuppression of a cell in a tumor microenvironment when expressed on a surface of the cell.

57. The polynucleotide of claim 56, wherein the armoring molecule comprises a dominant-negative TGF-β receptor type 2 (TGFβRIIDN).

58. The polynucleotide of claim 56 or 57, wherein the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105.

59. The polynucleotide of any one of claims 56 to 58, wherein the armoring molecule comprises the amino acid sequence set forth in SEQ ID NO: 105.

60. The polynucleotide of any one of claims 1 to 59, wherein the nucleotide sequence encoding the CAR has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 101 or 103.

61. The polynucleotide of claim 60, wherein the nucleotide sequence encoding the CAR comprises the nucleotide sequence set forth in SEQ ID NO: 101 or 103.

62. The polynucleotide of any one of claims 1 to 61, further comprising a second nucleotide sequence encoding an armoring molecule, wherein the second nucleotide sequence has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 104.

63. The polynucleotide of claim 62, wherein the second nucleotide comprises the nucleotide sequence set forth in SEQ ID NO: 104.

64. The polynucleotide of claim 63, wherein the nucleotide sequence encoding the CAR comprises the nucleotide sequence set forth in SEQ ID NO: 101, and the second nucleotide sequence comprises the nucleotide sequence set forth in SEQ ID NO: 104.

65. The polynucleotide of claim 63, wherein the nucleotide sequence encoding the CAR comprises the nucleotide sequence set forth in SEQ ID NO: 103, and the second nucleotide sequence comprises the nucleotide sequence set forth in SEQ ID NO: 104.

66. The polynucleotide of any one of claims 56 to 65, wherein the nucleotide sequence encoding the CAR and the second nucleotide sequence are linked by a third nucleotide sequence, wherein the third nucleotide sequence encodes a cleavable peptide linker.

67. The polynucleotide of claim 66, wherein the cleavable peptide linker is a self-cleaving peptide linker.

68. The polynucleotide of claim 66 or 67, wherein the cleavable peptide linker comprises a T2A peptide.

69. The polynucleotide of any one of claims 66-68, wherein the cleavable peptide linker comprises SEQ ID NO: 126.

70. The polynucleotide of any one of claims 1-17, 28, 31-43, 56-64, or 66-69, comprising a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 107.

71. The polynucleotide of any one of claims 1-17, 28, 31-43, 56-64 or 66-70, comprising the nucleotide sequence set forth in SEQ ID NO: 107.

72. The polynucleotide of any one of claims 1-17, 28, 31-43, 56-63 or 65-69, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 117.

73. The polynucleotide of any one of claims 1-17, 28, 31-43, 56-63, 65-69, or 72, wherein the CAR comprises the amino acid sequence set forth in SEQ ID NO: 117.

74. The polynucleotide of any one of claims 1-17, 28, 31-43, 56-59, or 66-69, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 120.

75. The polynucleotide of any one of claims 1-17, 28, 31-43, 56-59, 66-69, or 74, wherein the CAR comprises the amino acid sequence set forth in SEQ ID NO: 120.

76. The polynucleotide of any one of claims 1-17, 28, 31-43, 56-59, or 66-69, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 121.

77. The polynucleotide of any one of claims 1-17, 28, 31-43, 56-59, 66-69, or 76, wherein the CAR comprises the amino acid sequence set forth in SEQ ID NO: 121.

78. The polynucleotide of any one of claims 1-17, 28, 31-43, 56-59, or 66-69 wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 122.

79. The polynucleotide of any one of claims 1-17, 28, 31-43, 56-59, 66-69, or 78, wherein the CAR comprises the amino acid sequence set forth in SEQ ID NO: 122.

80. The polynucleotide of any one of claims 1-17, 28, 31-43, 56-59, 66-69, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 123.

81. The polynucleotide of any one of claims 1-17, 28, 31-43, 56-59, 66-69, or 80, wherein the CAR comprises the amino acid sequence set forth in SEQ ID NO: 123.

82. The polynucleotide of any one of claims 1-17, 28, 31-43, 56-59, or 66-69, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 124.

83. The polynucleotide of any one of claims 1-17, 28, 31-43, 56-59, 66-69, or 82, wherein the CAR comprises the amino acid sequence set forth in SEQ ID NO: 124.

84. The polynucleotide of any one of claims 1-17, 28, 31-43, 56-59, or 66-69, wherein the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 125.

85. The polynucleotide of any one of claims 1-17, 28, 31-43, 56-59, 66-69, or 84, wherein the CAR comprises the amino acid sequence set forth in SEQ ID NO: 125.

86. A vector or a set of vectors comprising the polynucleotide of any one of claims 1 to 85.

87. The vector or the set of vectors of claim 86, which is a viral vector.

88. A cell comprising the polynucleotide of any one of claims 1 to 85 or the vector or the set of vectors of claim 86 or 87.

89. The cell of claim 88, which is an immune cell.

90. The cell of claim 88 or 89, which is selected from the group consisting of a T cell, a Natural Killer (NK) cell, a cytotoxic T lymphocyte (CTL), a regulatory T cell, a tumor infiltrating lymphocyte, and any combination thereof.

91. A cell comprising (i) a polynucleotide encoding a chimeric antigen receptor (CAR) that binds human STEAP2 and (ii) a polynucleotide encoding an armoring molecule.

92. The cell of claim 91, which is an immune cell.

93. The cell of claim 91 or 92, which is selected from the group consisting of a T cell, a Natural Killer (NK) cell, a cytotoxic T lymphocyte (CTL), a regulatory T cell, a tumor infiltrating lymphocyte, and any combination thereof.

94. The cell of any one of claims 91-93, which is a human cell.

95. The cell of any one of claims 91-94, wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein
(a) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6;
(b) the VL-CDR1 comprises the amino acid sequence set forth in SEQ ID NO: 11, the VL-CDR2 comprises the amino acid sequence set forth in SEQ ID NO: 12, the VL-CDR3 comprises the amino acid sequence set forth in SEQ ID NO: 13, the VH-CDR1 comprises the amino acid sequence set forth in SEQ ID NO: 14, the VH-CDR2 comprises the amino acid sequence set forth in SEQ ID NO: 15, and the VH-CDR3 comprises the amino acid sequence set forth in SEQ ID NO: 16;
(c) the VL-CDR1 comprises the amino acid sequence set forth in SEQ ID NO: 21, the VL-CDR2 comprises the amino acid sequence set forth in SEQ ID NO: 22, the VL-CDR3 comprises the amino acid sequence set forth in SEQ ID NO: 23, the VH-CDR1 comprises the amino acid sequence set forth in SEQ ID NO: 24, the VH-CDR2 comprises the amino acid sequence set forth in SEQ ID NO: 25, and the VH-CDR3 comprises the amino acid sequence set forth in SEQ ID NO: 26;
(d) the VL-CDR1 comprises the amino acid sequence set forth in SEQ ID NO: 31, the VL-CDR2 comprises the amino acid sequence set forth in SEQ ID NO: 32, the VL-CDR3 comprises the amino acid sequence set forth in SEQ ID NO: 33, the VH-CDR1 comprises the amino acid sequence set forth in SEQ ID NO: 34, the VH-CDR2 comprises the amino acid sequence set forth in SEQ ID NO: 35, and the VH-CDR3 comprises the amino acid sequence set forth in SEQ ID NO: 36;
(e) the VL-CDR1 comprises the amino acid sequence set forth in SEQ ID NO: 41, the VL-CDR2 comprises the amino acid sequence set forth in SEQ ID NO: 42, the VL-CDR3 comprises the amino acid sequence set forth in SEQ ID NO: 43, the VH-CDR1 comprises the amino acid sequence set forth in SEQ ID NO: 44, the VH-CDR2 comprises the amino acid sequence set forth in SEQ ID NO: 45, and the VH-CDR3 comprises the amino acid sequence set forth in SEQ ID NO: 46; or
(f) the VL-CDR1 comprises the amino acid sequence set forth in SEQ ID NO: 91, the VL-CDR2 comprises the amino acid sequence set forth in SEQ ID NO: 92, the VL-CDR3 comprises the amino acid sequence set forth in SEQ ID NO: 93, the VH-CDR1 comprises the amino acid sequence set forth in SEQ ID NO: 94, the VH-CDR2 comprises the amino acid sequence set forth in SEQ ID NO: 95, and the VH-CDR3 comprises the amino acid sequence set forth in SEQ ID NO: 96.

96. The cell of any one of claims 91-95, wherein
(a) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38;
(e) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48;
(f) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58;
(g) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68;
(h) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78;
(i) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88; or
(j) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

97. The cell of any one of claims 91-96, wherein:
(a) the VH comprises the amino acid sequence set forth in SEQ ID NO: 7, and the VL comprises the amino acid sequence set forth in SEQ ID NO: 8;
(b) the VH comprises the amino acid sequence set forth in SEQ ID NO: 17, and the VL comprises the amino acid sequence set forth in SEQ ID NO: 18;
(c) the VH comprises the amino acid sequence set forth in SEQ ID NO: 27, and the VL comprises the amino acid sequence set forth in SEQ ID NO: 28;
(d) the VH comprises the amino acid sequence set forth in SEQ ID NO: 37, and the VL comprises the amino acid sequence set forth in SEQ ID NO: 38;
(e) the VH comprises the amino acid sequence set forth in SEQ ID NO: 47, and the VL comprises the amino acid sequence set forth in SEQ ID NO: 48;
(f) the VH comprises the amino acid sequence set forth in SEQ ID NO: 57, and the VL comprises the amino acid sequence set forth in SEQ ID NO: 58;
(g) the VH comprises the amino acid sequence set forth in SEQ ID NO: 67, and the VL comprises the amino acid sequence set forth in SEQ ID NO: 68;
(h) the VH comprises the amino acid sequence set forth in SEQ ID NO: 77, and the VL comprises the amino acid sequence set forth in SEQ ID NO: 78; or
(i) the VH comprises the amino acid sequence set forth in SEQ ID NO: 87, and the VL comprises the amino acid sequence set forth in SEQ ID NO: 88; or
(j) the VH comprises the amino acid sequence set forth in SEQ ID NO: 97, and the VL comprises the amino acid sequence set forth in SEQ ID NO: 98.

98. The cell of any one of claims 91-97, wherein the armoring molecule comprises a dominant-negative TGF-β receptor type 2 (TGFβRIIDN).

99. The cell of any one of claims 91-98, wherein the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105.

100. The cell of any one of claims 91-99, wherein the armoring molecule comprises the amino acid sequence set forth in SEQ ID NO: 105.

101. The cell of any one of claims 91-100, wherein the polynucleotide encoding the CAR comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 101 or 103.

102. The cell of claim 101, wherein the polynucleotide encoding the CAR comprises the nucleotide sequence set forth in SEQ ID NO: 101 or 103.

103. The cell of any one of claims 91-102, wherein the polynucleotide encoding the armoring molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 104.

104. The cell of any one of claims 91-103, wherein the polynucleotide encoding the armoring molecule comprises the nucleotide sequence set forth in SEQ ID NO: 104.

105. The cell of any one of claims 91-104, wherein the polynucleotide encoding the CAR comprises the nucleotide sequence set forth in SEQ ID NO: 101, and the polynucleotide encoding the armoring molecule comprises the nucleotide sequence set forth in SEQ ID NO: 104.

106. The cell of any one of claims 91-105, wherein the polynucleotide encoding the CAR comprises the nucleotide sequence set forth in SEQ ID NO: 103, and the polynucleotide encoding the armoring molecule comprises the nucleotide sequence set forth in SEQ ID NO: 104.

107. The cell of any one of claims 91 to 106, wherein the polynucleotide encoding the CAR and the polynucleotide encoding the armoring molecule are operably linked under the control of a single promoter.

108. The cell of any one of claims 91-107, wherein the polynucleotide encoding the CAR and the polynucleotide encoding the armoring molecule are operably linked by an IRES.

109. The cell of any one of claims 91-107, wherein the polynucleotide encoding the CAR and the polynucleotide encoding the armoring molecule are linked by a nucleotide sequence encoding a cleavable peptide linker.

110. The cell of claim 109, wherein the cleavable peptide linker is a self-cleaving peptide linker.

111. The cell of claim 109 or 110, wherein the cleavable peptide linker comprises a T2A peptide.

112. The cell of any one of claims 109-111, wherein the cleavable peptide linker comprises SEQ ID NO: 126.

113. A cell comprising a chimeric antigen receptor (CAR) that binds human STEAP2, wherein the CAR comprises an antigen-binding domain that comprises:
a) a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6;
b) a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16;
c) a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26;
d) a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36; or
e) a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46; or
f) a VL-CDR1 comprising the amino acid sequence set forth in SEQ ID NO: 91, a VL-CDR2 comprising the amino acid sequence set forth in SEQ ID NO: 92, a VL-CDR3 comprising the amino acid sequence set forth in SEQ ID NO: 93, a VH-CDR1 comprising the amino acid sequence set forth in SEQ ID NO: 94, a VH-CDR2 comprising the amino acid sequence set forth in SEQ ID NO: 45, a VH-CDR3 comprising the amino acid sequence set forth in SEQ ID NO: 96.

114. The cell of claim 113, further comprising an armoring molecule.

115. The cell of claim 113 or 114, wherein the CAR comprises an antigen-binding domain that comprises:
a) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
b) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
c) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
d) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38;
e) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48; or
f) a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

116. The cell of any one of claims 113-115, wherein the CAR comprises an antigen-binding domain that comprises the amino acid sequence of SEQ ID NO: 9.

117. The cell of any one of claims 113-115, wherein the CAR comprises an antigen-binding domain that comprises the amino acid sequence of SEQ ID NO: 99.

118. The cell of any one of claims 113-115, wherein the CAR comprises an amino acid sequence that comprises the amino acid sequence of SEQ ID NO: 10.

119. The cell of any one of claims 113-115, wherein the CAR comprises an amino acid sequence that comprises the amino acid sequence of SEQ ID NO: 108.

120. The cell of any one of claims 113-115, wherein the CAR comprises an amino acid sequence that comprises the amino acid sequence of SEQ ID NO: 109.

121. The cell of any one of claims 113-115, wherein the CAR comprises an amino acid sequence that comprises the amino acid sequence of SEQ ID NO: 110.

122. The cell of any one of claims 113-115, wherein the CAR comprises an amino acid sequence that comprises the amino acid sequence of SEQ ID NO: 111.

123. The cell of any one of claims 113-115, wherein the CAR comprises an amino acid sequence that comprises the amino acid sequence of SEQ ID NO: 112.

124. The cell of any one of claims 113-115, wherein the CAR comprises an amino acid sequence that comprises the amino acid sequence of SEQ ID NO: 113.

125. The cell of any one of claims 113-115, wherein the CAR comprises an amino acid sequence that comprises the amino acid sequence of SEQ ID NO: 114.

126. The cell of any one of claims 113-115, wherein the CAR comprises an amino acid sequence that comprises the amino acid sequence of SEQ ID NO: 115.

127. The cell of any one of claims 113-115, wherein the CAR comprises an amino acid sequence that comprises the amino acid sequence of SEQ ID NO: 116.

128. The cell of any one of claims 113-115, wherein the CAR comprises an amino acid sequence that comprises the amino acid sequence of SEQ ID NO: 118.

129. The cell of any one of claims 113-115, wherein the CAR comprises an amino acid sequence that comprises the amino acid sequence of SEQ ID NO: 119.

130. The cell of any one of claims 113-129, wherein the armoring molecule comprises a dominant-negative TGF-β receptor type 2 (TGFβRIIDN).

131. The cell of any one of claims 113-130, wherein the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105.

132. The cell of any one of claims 113-131, wherein the armoring molecule comprises the amino acid sequence set forth in SEQ ID NO: 105.

133. The cell of any one of claims 113-115 or 130-132, wherein the cell comprises an amino acid sequence that comprises the amino acid sequence set forth in SEQ ID NO: 106.

134. The cell of any one of claims 113-115 or 130-132, wherein the cell comprises an amino acid sequence that comprises the amino acid sequence set forth in SEQ ID NO: 117.

135. The cell of any one of claims 113-115 or 130-132, wherein the cell comprises an amino acid sequence that comprises the amino acid sequence set forth in SEQ ID NO: 120.

136. The cell of any one of claims 113-115 or 130-132, wherein the cell comprises an amino acid sequence that comprises the amino acid sequence set forth in SEQ ID NO: 121.

137. The cell of any one of claims 113-115 or 130-132, wherein the cell comprises an amino acid sequence that comprises the amino acid sequence set forth in SEQ ID NO: 122.

138. The cell of any one of claims 113-115 or 130-132, wherein the cell comprises an amino acid sequence that comprises the amino acid sequence set forth in SEQ ID NO: 123.

139. The cell of any one of claims 113-115 or 130-132, wherein the cell comprises an amino acid sequence that comprises the amino acid sequence set forth in SEQ ID NO: 124.

140. The cell of any one of claims 113-115 or 130-132, wherein the cell comprises an amino acid sequence that comprises the amino acid sequence set forth in SEQ ID NO: 125.

141. An antibody or an antigen-binding portion thereof that specifically binds human STEAP2, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein
(a) the VL-CDR1 comprises an amino acid sequence selected from SEQ ID NOs: 1, 11, 21, 31, 41, and 91;
(b) the VL-CDR2 comprises an amino acid sequence selected from SEQ ID NOs: 2, 12, 22, 32, 42, and 92;
(c) the VL-CDR3 comprises an amino acid sequence selected from SEQ ID NOs: 3, 13, 23, 33, 43, and 93;
(d) the VH-CDR1 comprises an amino acid sequence selected from SEQ ID NOs: 4, 14, 24, 34, 44, and 94;
(e) the VH-CDR2 comprises an amino acid sequence selected from SEQ ID NOs: 5, 15, 25, 35, 45, and 95; and
(f) the VH-CDR3 comprises an amino acid sequence selected from SEQ ID NOs: 6, 16, 26, 36, 46, and 96.

142. The antibody or an antigen-binding portion thereof of claim 141, wherein:
(a) the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6;
(b) the VL-CDR1 comprises the amino acid sequence set forth in SEQ ID NO: 11, the VL-CDR2 comprises the amino acid sequence set forth in SEQ ID NO: 12, the VL-CDR3 comprises the amino acid sequence set forth in SEQ ID NO: 13, the VH-CDR1 comprises the amino acid sequence set forth in SEQ ID NO: 14, the VH-CDR2 comprises the amino acid sequence set forth in SEQ ID NO: 15, and the VH-CDR3 comprises the amino acid sequence set forth in SEQ ID NO: 16;
(c) the VL-CDR1 comprises the amino acid sequence set forth in SEQ ID NO: 21, the VL-CDR2 comprises the amino acid sequence set forth in SEQ ID NO: 22, the VL-CDR3 comprises the amino acid sequence set forth in SEQ ID NO: 23, the VH-CDR1 comprises the amino acid sequence set forth in SEQ ID NO: 24, the VH-CDR2 comprises the amino acid sequence set forth in SEQ ID NO: 25, and the VH-CDR3 comprises the amino acid sequence set forth in SEQ ID NO: 26;
(d) the VL-CDR1 comprises the amino acid sequence set forth in SEQ ID NO: 31, the VL-CDR2 comprises the amino acid sequence set forth in SEQ ID NO: 32, the VL-CDR3 comprises the amino acid sequence set forth in SEQ ID NO: 33, the VH-CDR1 comprises the amino acid sequence set forth in SEQ ID NO: 34, the VH-CDR2 comprises the amino acid sequence set forth in SEQ ID NO: 35, and the VH-CDR3 comprises the amino acid sequence set forth in SEQ ID NO: 36;
(e) the VL-CDR1 comprises the amino acid sequence set forth in SEQ ID NO: 41, the VL-CDR2 comprises the amino acid sequence set forth in SEQ ID NO: 42, the VL-CDR3 comprises the amino acid sequence set forth in SEQ ID NO: 43, the VH-CDR1 comprises the amino acid sequence set forth in SEQ ID NO: 44, the VH-CDR2 comprises the amino acid sequence set forth in SEQ ID NO: 45, and the VH-CDR3 comprises the amino acid sequence set forth in SEQ ID NO: 46; or
(f) the VL-CDR1 comprises the amino acid sequence set forth in SEQ ID NO: 91, the VL-CDR2 comprises the amino acid sequence set forth in SEQ ID NO: 92, the VL-CDR3 comprises the amino acid sequence set forth in SEQ ID NO: 93, the VH-CDR1 comprises the amino acid sequence set forth in SEQ ID NO: 94, the VH-CDR2 comprises the amino acid sequence set forth in SEQ ID NO: 95, and the VH-CDR3 comprises the amino acid sequence set forth in SEQ ID NO: 96.

143. The antibody or an antigen-binding portion thereof of claim 141 or 142, wherein the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, 47, 57, 67, 77, 87, and 97.

144. The antibody or an antigen-binding portion thereof of any one of claims 141-143, wherein the VH comprises an amino acid sequence selected from SEQ ID NOs: 7, 17, 27, 37, 47, 57, 67, 77, 87, and 97.

145. The antibody or an antigen-binding portion thereof of any one of claims 141-144, wherein the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, 48, 58, 68, 78, 88, and 98.

146. The antibody or an antigen-binding portion thereof of any one of claims 141-145, wherein the VL comprises an amino acid sequence selected from SEQ ID NOs: 8, 18, 28, 38, 48, 58, 68, 78, 88, and 98.

147. The antibody or an antigen-binding portion thereof of any one of claims 141-146, wherein:
(a) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38;
(e) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48;
(f) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 57, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 58;
(g) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 67, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 68;
(h) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 77, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 78;
(i) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 87, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 88; or
(j) the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 97, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 98.

148. The antibody or an antigen-binding portion thereof of any one of claims 141-147, wherein:
(a) the VH comprises the amino acid sequence set forth in SEQ ID NO: 7, and the VL comprises the amino acid sequence set forth in SEQ ID NO: 8;
(b) the VH comprises the amino acid sequence set forth in SEQ ID NO: 17, and the VL comprises the amino acid sequence set forth in SEQ ID NO: 18;
(c) the VH comprises the amino acid sequence set forth in SEQ ID NO: 27, and the VL comprises the amino acid sequence set forth in SEQ ID NO: 28;
(d) the VH comprises the amino acid sequence set forth in SEQ ID NO: 37, and the VL comprises the amino acid sequence set forth in SEQ ID NO: 38;
(e) the VH comprises the amino acid sequence set forth in SEQ ID NO: 47, and the VL comprises the amino acid sequence set forth in SEQ ID NO: 48;
(f) the VH comprises the amino acid sequence set forth in SEQ ID NO: 57, and the VL comprises the amino acid sequence set forth in SEQ ID NO: 58;
(g) the VH comprises the amino acid sequence set forth in SEQ ID NO: 67, and the VL comprises the amino acid sequence set forth in SEQ ID NO: 68;
(h) the VH comprises the amino acid sequence set forth in SEQ ID NO: 77, and the VL comprises the amino acid sequence set forth in SEQ ID NO: 78;
(i) the VH comprises the amino acid sequence set forth in SEQ ID NO: 87, and the VL comprises the amino acid sequence set forth in SEQ ID NO: 88; or
(j) the VH comprises the amino acid sequence set forth in SEQ ID NO: 97, and the VL comprises the amino acid sequence set forth in SEQ ID NO: 98.

149. A pharmaceutical composition comprising the polynucleotide of any one of claims 1 to 85, the vector of claim 86 or 87, the cell of any one of claims 88 to 140, or the antibody or the antigen-binding portion thereof of any one of claims 141 to 148, and a pharmaceutically acceptable excipient.

150. A method of treating a disease or condition in a subject in need thereof, comprising administering to the subject the polynucleotide of any one of claims 1 to 85, the vector of claim 86 or 87, the cell of any one of claims 88 to 140, the antibody or the antigen-binding portion thereof of any one of claims 141 to 148, or the pharmaceutical composition of claim 149.

151. The method of claim 150, wherein the disease or condition comprises a cancer.

152. A method of treating a cancer in a subject in need thereof, comprising administering to the subject the polynucleotide of any one of claims 1 to 85, the vector of claim 86 or 87, the cell of any one of claims 88 to 140, the antibody or the antigen-binding portion thereof of any one of claims 141 to 148, or the pharmaceutical composition of claim 149.

153. The method of claim 151 or 152, wherein the cancer comprises a prostate cancer.

154. The method of claim 153, wherein the prostate cancer is metastatic, recurrent, or relapsed.

155. Use of the polynucleotide of any one of claims 1 to 85, the vector of claim 86 or 87, the cell of any one of claims 88 to 140, the antibody or the antigen-binding portion thereof of any one of claims 141 to 148, or the pharmaceutical composition of claim 149 in the treatment of a disease or condition in a subject in need thereof.

156. The use of claim 155, wherein the disease or condition comprises a cancer.

157. Use of the polynucleotide of any one of claims 1 to 85, the vector of claim 86 or 87, the cell of any one of claims 88 to 140, the antibody or the antigen-binding portion thereof of any one of claims 141 to 148, or the pharmaceutical composition of claim 149 in the treatment of a cancer in a subject in need thereof.

158. The use of claim 156 or 157, wherein the cancer comprises a prostate cancer.

159. The use of claim 158, wherein the prostate cancer is metastatic, recurrent, or relapsed.


SEQUENCE LISTING (133 sequences)

SEQ ID NO: 1 - protein, 11 aa, synthetic construct
  REGION 1..11: note = STEAP2 VL CDR1 (40A3)
RASQSVNSNLA

SEQ ID NO: 2 - protein, 7 aa, synthetic construct
  REGION 1..7: note = STEAP2 VL CDR2
GASTRAT

SEQ ID NO: 3 - protein, 9 aa, synthetic construct
  REGION 1..9: note = STEAP2 VL CDR3
QQYNNWPFT

SEQ ID NO: 4 - protein, 7 aa, synthetic construct
  REGION 1..7: note = STEAP2 VH CDR1
RNSAVWN

SEQ ID NO: 5 - protein, 18 aa, synthetic construct
  REGION 1..18: note = STEAP2 VH CDR2
RTYYRSKWYNDYAVSVKS

SEQ ID NO: 6 - protein, 13 aa, synthetic construct
  REGION 1..13: note = STEAP2 VHCDR3
GLLQNNFYYYMDV

SEQ ID NO: 7 - protein, 125 aa, synthetic construct
  REGION 1..125: note = STEAP2 VH
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 8 - protein, 107 aa, synthetic construct
  REGION 1..107: note = STEAP2 VL
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIK

SEQ ID NO: 9 - protein, 252 aa, synthetic construct
  REGION 1..252: note = STEAP2 scFv amino acid sequence
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 10 - protein, 469 aa, synthetic construct
  REGION 1..469: note = STEAP2 BZ CAR amino acid sequence
MLLLVTSLLLCELPHPAFLLIPEIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 11 aa, synthetic construct
  REGION 1..11: note = STEAP2-3 VL CDR1 (40A3GL-LO7)
RASQSVSSNLA

SEQ ID NO: 12 - protein, 7 aa, synthetic construct
  REGION 1..7: note = STEAP2-3 VL CDR2
GASTRAT

SEQ ID NO: 13 - protein, 9 aa, synthetic construct
  REGION 1..9: note = STEAP2-3 VL CDR3
QQYNNWPFT

SEQ ID NO: 14 - protein, 7 aa, synthetic construct
  REGION 1..7: note = STEAP2-3 VH CDR1
RNSAVWN

SEQ ID NO: 15 - protein, 18 aa, synthetic construct
  REGION 1..18: note = STEAP2-3 VH CDR2
RTYYRSKWYNDYAVSVKS

SEQ ID NO: 16 - protein, 13 aa, synthetic construct
  REGION 1..13: note = STEAP2-3 VH CDR3
GLLQNQFYYYMDV

SEQ ID NO: 17 - protein, 125 aa, synthetic construct
  REGION 1..125: note = STEAP2-3 VH
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSS

SEQ ID NO: 18 - protein, 107 aa, synthetic construct
  REGION 1..107: note = STEAP2-3 VL
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 19-20 - skipped (2 entries)

SEQ ID NO: 21 - protein, 16 aa, synthetic construct
  REGION 1..16: note = STEAP2-2 VL CDR1(mm30D12)
RSSQSVVHSNGNTYLE

SEQ ID NO: 22 - protein, 7 aa, synthetic construct
  REGION 1..7: note = STEAP2-2 VL CDR2
KVSNRFS

SEQ ID NO: 23 - protein, 9 aa, synthetic construct
  REGION 1..9: note = STEAP2-2 VL CDR3
FQGSHVPYT

SEQ ID NO: 24 - protein, 5 aa, synthetic construct
  REGION 1..5: note = STEAP2-2 VH CDR1
SYGMS

SEQ ID NO: 25 - protein, 17 aa, synthetic construct
  REGION 1..17: note = STEAP2-2 VH CDR2
TISSGGSYTFYPDIMKG

SEQ ID NO: 26 - protein, 13 aa, synthetic construct
  REGION 1..13: note = STEAP2-2 VH CDR3
RGYGTIYTFSFDS

SEQ ID NO: 27 - protein, 122 aa, synthetic construct
  REGION 1..122: note = STEAP2-2 VH
EVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRGYGTIYTFSFDSWGQGTTLTVSS

SEQ ID NO: 28 - protein, 112 aa, synthetic construct
  REGION 1..112: note = STEAP2-2 VL
DVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 29-30 - skipped (2 entries)

SEQ ID NO: 31 - protein, 16 aa, synthetic construct
  REGION 1..16: note = STEAP2-2 Humanized1 VL CDR1
RSSQSVVHSNANTYLE

SEQ ID NO: 32 - protein, 7 aa, synthetic construct
  REGION 1..7: note = STEAP2-2 Humanized1 VL CDR2
KVSNRFS

SEQ ID NO: 33 - protein, 9 aa, synthetic construct
  REGION 1..9: note = STEAP2-2 Humanized1 VL CDR3
FQGSHVPYT

SEQ ID NO: 34 - protein, 5 aa, synthetic construct
  REGION 1..5: note = STEAP2-2 Humanized1 VH CDR1
SYGMS

SEQ ID NO: 35 - protein, 17 aa, synthetic construct
  REGION 1..17: note = STEAP2-2 Humanized1 VH CDR2
TISSGGSYTFYPDIMKG

SEQ ID NO: 36 - protein, 13 aa, synthetic construct
  REGION 1..13: note = STEAP2-2 Humanized1 VH CDR3
RGYGTIYTFSFDA

SEQ ID NO: 37 - protein, 122 aa, synthetic construct
  REGION 1..122: note = STEAP2-2 Humanized1 VH
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSS

SEQ ID NO: 38 - protein, 112 aa, synthetic construct
  REGION 1..112: note = STEAP2-2 Humanized1 VL
DVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIK

SEQ ID NO: 39-40 - skipped (2 entries)

SEQ ID NO: 41 - protein, 16 aa, synthetic construct
  REGION 1..16: note = STEAP2-2 Humanized2 VL CDR1
RSSQSVVHSNANTYLE

SEQ ID NO: 42 - protein, 7 aa, synthetic construct
  REGION 1..7: note = STEAP2-2 Humanized2 VL CDR2
KVSNRFS

SEQ ID NO: 43 - protein, 9 aa, synthetic construct
  REGION 1..9: note = STEAP2-2 Humanized2 VL CDR3
FQGSHVPYT

SEQ ID NO: 44 - protein, 5 aa, synthetic construct
  REGION 1..5: note = STEAP2-2 Humanized2 VH CDR1
SYGMS

SEQ ID NO: 45 - protein, 17 aa, synthetic construct
  REGION 1..17: note = STEAP2-2 Humanized2 VH CDR2
TISSGGSYTFYPDIMKG

SEQ ID NO: 46 - protein, 13 aa, synthetic construct
  REGION 1..13: note = STEAP2-2 Humanized2 VH CDR3
RGYGTIYTFSFDA

SEQ ID NO: 47 - protein, 122 aa, synthetic construct
  REGION 1..122: note = STEAP2-2 Humanized2 VH
EVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRGYGTIYTFSFDAWGQGTTLTVSS

SEQ ID NO: 48 - protein, 112 aa, synthetic construct; identical to SEQ ID NO: 38
  REGION 1..112: note = STEAP2-2 Humanized2 VL

SEQ ID NO: 49-56 - skipped (8 entries)

SEQ ID NO: 57 - protein, 120 aa, synthetic construct
  REGION 1..120: note = STEAP2-4 VH (40A1)
EVQLVESGGGLVQPGGSLRLSCAASGFTFSSFAMTWVRQAPGKGLEWVSVITYSGGRTYYADSVKGRFTISRDNSKNTLYLQMNSLRAEDTAVYFCAKDRIAAVGPFDYWGQGTLVTVSS

SEQ ID NO: 58 - protein, 107 aa, synthetic construct
  REGION 1..107: note = STEAP2-4 VL
DIQLTQSPSFLSASVGDRVTITCRASQGISVYLAWYQQEPGKAPKLLIYAASTLQSGVPSRFSGSGSGTEFTLTISSLQPEDFATYYCQQLNSYPRTFGQGTKVEIK

SEQ ID NO: 59-66 - skipped (8 entries)

SEQ ID NO: 67 - protein, 116 aa, synthetic construct
  REGION 1..116: note = STEAP2-5 VH (34C1)
QVQLVQSGAEVKKPGASVKVSCKASGYTFTSYGISWVRQAPGQGLEWMGWISGYTGNTNYAQKLQGRVTMTADTSTSTAYMELRSLRSDDTAVYYCARGGSYFDYWGQGTLVTVSS

SEQ ID NO: 68 - protein, 108 aa, synthetic construct
  REGION 1..108: note = STEAP2-5 VL
DIQMTQSPSTLSASVGDRVTITCRASQSISRWLAWYQQKPGKAPKLLIYKASSLESGVPSRFSGSGSGTEFTLTISSLQPDDFATYYCQQFNSFSPITFGQGTRLEIK

SEQ ID NO: 69-76 - skipped (8 entries)

SEQ ID NO: 77 - protein, 117 aa, synthetic construct
  REGION 1..117: note = STEAP2-6 VH (6E10)
QVQLQQPGAELVKPGASVKLSCKASGYTFTSYWMEWVKQRPGQGLEWIGMIHPNSGITNYNERFKNKATLTVDKSSSTAYMQLSSLTSEDSAVYYCARDHYYILAYWGQGTLVTVSA

SEQ ID NO: 78 - protein, 112 aa, synthetic construct; identical to SEQ ID NO: 28
  REGION 1..112: note = STEAP2-6 VL

SEQ ID NO: 79-86 - skipped (8 entries)

SEQ ID NO: 87 - protein, 120 aa, synthetic construct
  REGION 1..120: note = STEAP2-7 VH (22F3)
QVQLQQPGADLVKPGASVKMSCKASGHTFTNYWVTWVKQRPGQGLEWIGNFYPGSGIIKYNENFRSKATLTVDISSSTAYMQLSSLTSEDSAVYYCARSKLGDSFYFDYWGQGTTLTVSS

SEQ ID NO: 88 - protein, 112 aa, synthetic construct
  REGION 1..112: note = STEAP2-7 VL
DVVMTQTPLSLPVSLGNQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEIK

SEQ ID NO: 89-90 - skipped (2 entries)

SEQ ID NO: 91 - protein, 11 aa, synthetic construct
  REGION 1..11: note = STEAP2-840A3GL- LO14)VL CDR1
RASQSVASNLA

SEQ ID NO: 92 - protein, 7 aa, synthetic construct
  REGION 1..7: note = STEAP2-8 VL CDR2
GASTRAT

SEQ ID NO: 93 - protein, 9 aa, synthetic construct
  REGION 1..9: note = STEAP2-8 VL CDR3
QQYNNWPFT

SEQ ID NO: 94 - protein, 7 aa, synthetic construct
  REGION 1..7: note = STEAP2-8 VH CDR1
RNSAVWN

SEQ ID NO: 95 - protein, 18 aa, synthetic construct
  REGION 1..18: note = STEAP2-8 VH CDR2
RTYYRSKWYNDYAPSVKS

SEQ ID NO: 96 - protein, 13 aa, synthetic construct
  REGION 1..13: note = STEAP2-8 VH CDR3
GLRQNQFYYYMDV

SEQ ID NO: 97 - protein, 125 aa, synthetic construct
  REGION 1..125: note = STEAP2-8 VH
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSS

SEQ ID NO: 98 - protein, 107 aa, synthetic construct
  REGION 1..107: note = STEAP2-8 VL
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIK

SEQ ID NO: 99 - protein, 252 aa, synthetic construct
  REGION 1..252: note = STEAP2-8 scFv
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSS

SEQ ID NO: 100 - skipped

SEQ ID NO: 101 - DNA, 756 bp, synthetic construct
  misc_feature 1..756: note = STEAP2 scFv nucleic acid sequence
  source 1..756: mol_type = other DNA
gagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctcc

SEQ ID NO: 102 - DNA, 1410 bp, synthetic construct
  misc_feature 1..1410: note = STEAP2 BZ CAR nucleic acid sequence
  source 1..1410: mol_type = other DNA
atgctgctccttgtcacatcactgctgctctgcgaactgccccaccctgcattcctcctgatccccgagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctccggctccgaatcaaaatacggtccgccatgcccaccgtgccccttctgggtgctcgtggtcgtcggaggggttctggcctgctactccctgctggtcaccgtggcgtttatcatcttctgggtgaagcggggaaggaagaagctactgtacattttcaagcagcctttcatgcggcctgtgcagaccacccaggaagaggacggctgttcctgccggttccccgaggaagaggaagggggttgcgagctgcgcgtgaagttcagcaggagcgctgatgccccagcgtaccaacaggggcaaaaccagttgtacaacgaactgaaccttggtcggcgcgaagagtacgacgtgcttgacaagcgccgcggcagagatcccgagatgggtggaaagccgcggcggaagaatccgcaggaagggctctacaacgagctccagaaggacaagatggccgaagcctacagcgaaatcgggatgaagggcgaaagacgccggggaaaaggacacgacggactgtaccaggggttgtcgaccgcgaccaaggacacctacgacgccctgcatatgcaagccttgccgccgagatga

SEQ ID NO: 103 - DNA, 762 bp, synthetic construct
  misc_feature 1..762: note = STEAP2-2 scFv nucleic acid sequence
  source 1..762: mol_type = other DNA
gatgttttgatgacccaaactcctctctccctgcctgtcagtcttggagatcaagcctccatctcttgcagatctagtcagagtgttgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaaggaggcggaggatctggtggtggtggatctggcggcggaggaagtggtggcggaggctctgaggtgcagctggtggagtctgggggagacttagtgaagcctggagggtccctgaaactctcctgtgccgcctctggattctctttctcctcttatggcatgtcttgggttcgccagactccagacaagaggctggaatgggtcgcaaccattagtagtggtggtagttacaccttctatcccgacattatgaaggggcgattcaccatctccagagacaatgccatgaacaccctgtacctgcaaatgagcagtctgaagtctgaggactcagccatgtattactgtgcaagacggggctacggtactatctacacgttttcctttgactcctggggccaaggcaccactctcacagtctccagc

SEQ ID NO: 104 - DNA, 582 bp, synthetic construct
  misc_feature 1..582: note = Dominant-negative TGF-? receptor type 2 nucleic acid sequence
  source 1..582: mol_type = other DNA
atgggacgcgggctgcttcgaggactctggccacttcatatcgtgttgtggactcgcatcgcttcaaccattccgccgcacgtgcagaagtccgtgaacaatgacatgatcgtgaccgacaacaacggtgcagtgaagttcccacagctgtgcaagttctgcgatgtcagattcagcacttgcgacaaccagaagtcctgcatgtcaaactgctccatcacctccatctgcgagaagcctcaagaggtctgcgtggccgtgtggcggaagaacgacgagaacatcaccctggaaaccgtgtgccacgatccgaagctgccttatcacgacttcattctggaagatgccgcctcgcccaagtgtatcatgaaagaaaagaaaaagcccggagaaacgttcttcatgtgctcgtgtagctccgacgagtgcaacgacaacattatctttagcgaagagtacaacacttccaaccctgacctcctgctcgtgatttttcaagtcaccggcatttccctgctgcccccgctgggcgtggcgatctcggtgatcattatcttctactgttaccgggtcaataggcag

SEQ ID NO: 105 - protein, 194 aa, synthetic construct
  REGION 1..194: note = Dominant-negative TGF-B receptor type 2 amino acid sequence
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 106 - protein, 684 aa, synthetic construct
  REGION 1..684: note = STEAP2 BZ CAR-T2A-Dominant-negative TGF-B receptor type 2aminoacid sequence
MLLLVTSLLLCELPHPAFLLIPEIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 107 - DNA, 2052 bp, synthetic construct
  misc_feature 1..2052: note = STEAP2 BZ CAR-T2A-Dominant-negative TGF-B receptor type 2nucleic acid sequence
  source 1..2052: mol_type = other DNA
atgctgctccttgtcacatcactgctgctctgcgaactgccccaccctgcattcctcctgatccccgagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctccggctccgaatcaaaatacggtccgccatgcccaccgtgccccttctgggtgctcgtggtcgtcggaggggttctggcctgctactccctgctggtcaccgtggcgtttatcatcttctgggtgaagcggggaaggaagaagctactgtacattttcaagcagcctttcatgcggcctgtgcagaccacccaggaagaggacggctgttcctgccggttccccgaggaagaggaagggggttgcgagctgcgcgtgaagttcagcaggagcgctgatgccccagcgtaccaacaggggcaaaaccagttgtacaacgaactgaaccttggtcggcgcgaagagtacgacgtgcttgacaagcgccgcggcagagatcccgagatgggtggaaagccgcggcggaagaatccgcaggaagggctctacaacgagctccagaaggacaagatggccgaagcctacagcgaaatcgggatgaagggcgaaagacgccggggaaaaggacacgacggactgtaccaggggttgtcgaccgcgaccaaggacacctacgacgccctgcatatgcaagccttgccgccgagaggatccggagaggggaggggaagcctcctcacttgcggcgatgtggaggaaaacccgggtcctatgggacgcgggctgcttcgaggactctggccacttcatatcgtgttgtggactcgcatcgcttcaaccattccgccgcacgtgcagaagtccgtgaacaatgacatgatcgtgaccgacaacaacggtgcagtgaagttcccacagctgtgcaagttctgcgatgtcagattcagcacttgcgacaaccagaagtcctgcatgtcaaactgctccatcacctccatctgcgagaagcctcaagaggtctgcgtggccgtgtggcggaagaacgacgagaacatcaccctggaaaccgtgtgccacgatccgaagctgccttatcacgacttcattctggaagatgccgcctcgcccaagtgtatcatgaaagaaaagaaaaagcccggagaaacgttcttcatgtgctcgtgtagctccgacgagtgcaacgacaacattatctttagcgaagagtacaacacttccaaccctgacctcctgctcgtgatttttcaagtcaccggcatttccctgctgcccccgctgggcgtggcgatctcggtgatcattatcttctactgttaccgggtcaataggcag

SEQ ID NO: 108 - protein, 471 aa, synthetic construct
  REGION 1..471: note = STEAP2-2 BZ CAR amino acid sequence
MLLLVTSLLLCELPHPAFLLIPDVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIKGGGGSGGGGSGGGGSGGGGSEVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRGYGTIYTFSFDSWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 471 aa, synthetic construct
  REGION 1..471: note = STEAP2-2Humanized1 BZ CAR amino acid sequence
MLLLVTSLLLCELPHPAFLLIPDVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIKGGGGSGGGGSGGGGSGGGGSEVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 471 aa, synthetic construct
  REGION 1..471: note = STEAP2-2 Humanized2 BZ CAR amino acid sequence
MLLLVTSLLLCELPHPAFLLIPDVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIKGGGGSGGGGSGGGGSGGGGSEVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRGYGTIYTFSFDAWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 469 aa, synthetic construct
  REGION 1..469: note = STEAP2-3 BZ CAR amino acid sequence
MLLLVTSLLLCELPHPAFLLIPEIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 464 aa, synthetic construct
  REGION 1..464: note = STEAP2-4 BZ CAR amino acid sequence
MLLLVTSLLLCELPHPAFLLIPDIQLTQSPSFLSASVGDRVTITCRASQGISVYLAWYQQEPGKAPKLLIYAASTLQSGVPSRFSGSGSGTEFTLTISSLQPEDFATYYCQQLNSYPRTFGQGTKVEIKGGGGSGGGGSGGGGSGGGGSEVQLVESGGGLVQPGGSLRLSCAASGFTFSSFAMTWVRQAPGKGLEWVSVITYSGGRTYYADSVKGRFTISRDNSKNTLYLQMNSLRAEDTAVYFCAKDRIAAVGPFDYWGQGTLVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 461 aa, synthetic construct
  REGION 1..461: note = STEAP2-5 BZ CAR amino acid sequence
MLLLVTSLLLCELPHPAFLLIPDIQMTQSPSTLSASVGDRVTITCRASQSISRWLAWYQQKPGKAPKLLIYKASSLESGVPSRFSGSGSGTEFTLTISSLQPDDFATYYCQQFNSFSPITFGQGTRLEIKGGGGSGGGGSGGGGSGGGGSQVQLVQSGAEVKKPGASVKVSCKASGYTFTSYGISWVRQAPGQGLEWMGWISGYTGNTNYAQKLQGRVTMTADTSTSTAYMELRSLRSDDTAVYYCARGGSYFDYWGQGTLVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 466 aa, synthetic construct
  REGION 1..466: note = STEAP2-6 BZ CAR amino acid sequence
MLLLVTSLLLCELPHPAFLLIPDVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIKGGGGSGGGGSGGGGSGGGGSQVQLQQPGAELVKPGASVKLSCKASGYTFTSYWMEWVKQRPGQGLEWIGMIHPNSGITNYNERFKNKATLTVDKSSSTAYMQLSSLTSEDSAVYYCARDHYYILAYWGQGTLVTVSAGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 469 aa, synthetic construct
  REGION 1..469: note = STEAP2-7 BZ CAR amino acid sequence
MLLLVTSLLLCELPHPAFLLIPDVVMTQTPLSLPVSLGNQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEIKGGGGSGGGGSGGGGSGGGGSQVQLQQPGADLVKPGASVKMSCKASGHTFTNYWVTWVKQRPGQGLEWIGNFYPGSGIIKYNENFRSKATLTVDISSSTAYMQLSSLTSEDSAVYYCARSKLGDSFYFDYWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 469 aa, synthetic construct
  REGION 1..469: note = STEAP2-8 BZ CAR amino acid sequence
MLLLVTSLLLCELPHPAFLLIPEIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 686 aa, synthetic construct
  REGION 1..686: note = STEAP2-2 BZ CAR-T2A-Dominant-negative TGF-? receptor type 2amino acid sequence
MLLLVTSLLLCELPHPAFLLIPDVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIKGGGGSGGGGSGGGGSGGGGSEVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRGYGTIYTFSFDSWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 118 - protein, 686 aa, synthetic construct
  REGION 1..686: note = STEAP2-2Humanized1 BZ CAR amino acid sequence
MLLLVTSLLLCELPHPAFLLIPDVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIKGGGGSGGGGSGGGGSGGGGSEVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 119 - protein, 686 aa, synthetic construct
  REGION 1..686: note = STEAP2-2 Humanized2 BZ CAR amino acid sequence
MLLLVTSLLLCELPHPAFLLIPDVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIKGGGGSGGGGSGGGGSGGGGSEVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRGYGTIYTFSFDAWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 120 - protein, 684 aa, synthetic construct
  REGION 1..684: note = STEAP2-3 BZ CAR-T2A-Dominant-negative TGF-B receptor type 2amino acid sequence
MLLLVTSLLLCELPHPAFLLIPEIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 121 - protein, 679 aa, synthetic construct
  REGION 1..679: note = STEAP2-4 BZ CAR-T2A-Dominant-negative TGF-B receptor type 2amino acid sequence
MLLLVTSLLLCELPHPAFLLIPDIQLTQSPSFLSASVGDRVTITCRASQGISVYLAWYQQEPGKAPKLLIYAASTLQSGVPSRFSGSGSGTEFTLTISSLQPEDFATYYCQQLNSYPRTFGQGTKVEIKGGGGSGGGGSGGGGSGGGGSEVQLVESGGGLVQPGGSLRLSCAASGFTFSSFAMTWVRQAPGKGLEWVSVITYSGGRTYYADSVKGRFTISRDNSKNTLYLQMNSLRAEDTAVYFCAKDRIAAVGPFDYWGQGTLVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 122 - protein, 676 aa, synthetic construct
  REGION 1..676: note = STEAP2-5 BZ CAR-T2A-Dominant-negative TGF-B receptor type 2amino acid sequence
MLLLVTSLLLCELPHPAFLLIPDIQMTQSPSTLSASVGDRVTITCRASQSISRWLAWYQQKPGKAPKLLIYKASSLESGVPSRFSGSGSGTEFTLTISSLQPDDFATYYCQQFNSFSPITFGQGTRLEIKGGGGSGGGGSGGGGSGGGGSQVQLVQSGAEVKKPGASVKVSCKASGYTFTSYGISWVRQAPGQGLEWMGWISGYTGNTNYAQKLQGRVTMTADTSTSTAYMELRSLRSDDTAVYYCARGGSYFDYWGQGTLVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 123 - protein, 681 aa, synthetic construct
  REGION 1..681: note = STEAP2-6 BZ CAR-T2A-Dominant-negative TGF-B receptor type 2amino acid sequence
MLLLVTSLLLCELPHPAFLLIPDVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIKGGGGSGGGGSGGGGSGGGGSQVQLQQPGAELVKPGASVKLSCKASGYTFTSYWMEWVKQRPGQGLEWIGMIHPNSGITNYNERFKNKATLTVDKSSSTAYMQLSSLTSEDSAVYYCARDHYYILAYWGQGTLVTVSAGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 124 - protein, 684 aa, synthetic construct
  REGION 1..684: note = STEAP2-7 BZ CAR-T2A-Dominant-negative TGF-B receptor type 2amino acid sequence
MLLLVTSLLLCELPHPAFLLIPDVVMTQTPLSLPVSLGNQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEIKGGGGSGGGGSGGGGSGGGGSQVQLQQPGADLVKPGASVKMSCKASGHTFTNYWVTWVKQRPGQGLEWIGNFYPGSGIIKYNENFRSKATLTVDISSSTAYMQLSSLTSEDSAVYYCARSKLGDSFYFDYWGQGTTLTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 125 - protein, 684 aa, synthetic construct
  REGION 1..684: note = STEAP2-8 BZ CAR-T2A-Dominant-negative TGF-B receptor type 2amino acid sequence
MLLLVTSLLLCELPHPAFLLIPEIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 126 - protein, 21 aa, synthetic construct
  REGION 1..21: note = T2A Peptide
GSGEGRGSLLTCGDVEENPGP

SEQ ID NO: 127 - protein, 22 aa, synthetic construct
  REGION 1..22: note = Signal Peptide
MLLLVTSLLLCELPHPAFLLIP

SEQ ID NO: 128 - protein, 12 aa, synthetic construct
  REGION 1..12: note = IgG4P Hinge
ESKYGPPCPPCP

SEQ ID NO: 129 - protein, 27 aa, synthetic construct
  REGION 1..27: note = CD28 Transmembrane Domain
FWVLVVVGGVLACYSLLVTVAFIIFWV

SEQ ID NO: 130 - protein, 42 aa, synthetic construct
  REGION 1..42: note = 4-1BB Activation Domain (B domain)
KRGRKKLLYIFKQPFMRPVQTTQEEDGCSCRFPEEEEGGCEL

SEQ ID NO: 131 - protein, 112 aa, synthetic construct
  REGION 1..112: note = CD3zeta Domain (z domain)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 154 aa, synthetic construct
  REGION 1..154: note = Bz Domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 41 aa, synthetic construct
RSKRSRLLHSDYMNMTPRRPGPTRKHYQPYAPPRDFAAYRS